SEQUENCE LISTING (46 sequences)

SEQ ID NO: 1 - DNA, 5498 bp, synthetic construct
  misc_feature 1..5498: note = Recombinant plasmid pAPSE10180; ErkA stem loop + capsid protein
  regulatory 24..42: note = promoter - bacteriophage T7 gene 1 promoter; regulatory_class = promoter
  misc_feature 127..145: note = bacteriophage MS2 pac sequence
  misc_feature 175..182: note = restriction endonuclease AsiSI recognition site
  misc_feature 179..384: note = ErkA sense strand gene fragment
  misc_feature 436..454: note = bacteriophage MS2 pac sequence
  misc_feature 504..511: note = restriction endonuclease NotI recognition site
  misc_feature 505..696: note = ErkA antisense strand gene fragment
  misc_feature 692..699: note = restriction endonuclease PacI recognition site
  misc_feature 709..727: note = bacteriophage MS2 pac sequence
  regulatory 743..790: note = terminator - bacteriophage T7 transcription terminator; regulatory_class = terminator
  regulatory 1116..1134: note = promoter - bacteriophage T7 gene 1 promoter; regulatory_class = promoter
  gene 1207..1596: note = bacteriophage MS2 coat protein gene
  regulatory 1448..1495: note = terminator - bacteriophage T7 transcription terminator; regulatory_class = terminator
  source 1..5498: mol_type = other DNA
ttcagatctcgatcccgcgaaattaatacgactcactatagggagaccacaacggtttccctctagatcacaagtttgtacaaaaaagcaggctaagaaggagatatacatacgccggccattcaaacatgaggattacccatgtaacctaaggccggtgtccaggcgcgctccgcgatcgcacgcggacaaagttcctcaatctaatgctgaagttataaggggacaaatatttgaagttggtcctaggtatattaaactcgcctatataggtgaaggagcttatggcatggttgtgtctgcggatgacacgctaacaaaccaaagagttgcaataaaaaaaatatcgccctttgaacaccaaacttattgctactacagtttaaacgcaatcgcagcaaactccggcatctactaatagacgccggccattcaacatgaggattacccatgtaacctaagaagacaacaaagaagttcaactctttatgtattgatcttccgcggccgccaataagtttggtgttcaaagggcgatattttttttattgcaactctttggtttgttagcgtgtcatccgcagacacaaccatgccataagctccttcacctatataggcgagtttaatatacctaggaccaacttcaaatatttgtccccttataacttcagcattagattgaggaactttaattaaggagttcaaacatgaggatcacccatgtcgaagctcccacaccctagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcgggcatgcatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatccagatctcgatcccgcgaaattaatacgactcactatagggagaccacaacggtttccctctagatcacaagtttgtacaaaaaagcaggctaagaaggagatatacatatggcgtctaactttacccaattcgttctggttgataacggcggtacgggtgacgttaccgtagctccgtccaacttcgccaacggtgttgcggaatggattagctctaacagccgctctcaggcctacaaagtcacgtgctccgttcgtcagtctagcgcgcagaatcgcaaatacaccatcaaagttgaagtaccgaaagtcgcaacgcagaccgtaggcggcgtagaactcccagttgcggcctggcgctcttacctcaacatggaactgactattccgatttttgcgacgaactccgactgcgaactgattgttaaggcaatgcagggcctgctgaaagacggtaatccgatcccatctgcaatcgctgctaactctggcatttactaataagcggacgcgctgccaccgctgagcaataactagcataaccccttggggcctctaaacgggtcttgaggggttttttgctgaaaggaggaactatatccggcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaa

SEQ ID NO: 2 - DNA, 4696 bp, synthetic construct
  misc_feature 1..4696: note = Recombinant plasmid pAPSE10181; ErkA stem loop - coat protein
  regulatory 24..42: note = promoter - bacteriophage T7 gene 1 promoter; regulatory_class = promoter
  misc_feature 127..145: note = bacteriophage MS2 pac sequence
  misc_feature 175..182: note = restriction endonuclease AsiSI recognition site
  misc_feature 179..384: note = ErkA sense strand gene fragment
  misc_feature 436..454: note = bacteriophage MS2 pac sequence
  misc_feature 504..511: note = restriction endonuclease NotI recognition site
  misc_feature 505..696: note = ErkA antisense strand gene fragment
  misc_feature 692..699: note = restriction endonuclease PacI recognition site
  misc_feature 709..727: note = bacteriophage MS2 pac sequence
  regulatory 743..790: note = terminator - bacteriophage T7 transcription terminator; regulatory_class = terminator
  misc_feature 897..902: note = restriction endonuclease SphI recognition site
  source 1..4696: mol_type = other DNA
ttcagatctcgatcccgcgaaattaatacgactcactatagggagaccacaacggtttccctctagatcacaagtttgtacaaaaaagcaggctaagaaggagatatacatacgccggccattcaaacatgaggattacccatgtaacctaaggccggtgtccaggcgcgctccgcgatcgcacgcggacaaagttcctcaatctaatgctgaagttataaggggacaaatatttgaagttggtcctaggtatattaaactcgcctatataggtgaaggagcttatggcatggttgtgtctgcggatgacacgctaacaaaccaaagagttgcaataaaaaaaatatcgccctttgaacaccaaacttattgctactacagtttaaacgcaatcgcagcaaactccggcatctactaatagacgccggccattcaacatgaggattacccatgtaacctaagaagacaacaaagaagttcaactctttatgtattgatcttccgcggccgccaataagtttggtgttcaaagggcgatattttttttattgcaactctttggtttgttagcgtgtcatccgcagacacaaccatgccataagctccttcacctatataggcgagtttaatatacctaggaccaacttcaaatatttgtccccttataacttcagcattagattgaggaactttaattaaggagttcaaacatgaggatcacccatgtcgaagctcccacaccctagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcgggcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaa

SEQ ID NO: 3 - DNA, 24 bp, synthetic construct
  misc_feature 1..24: note = synthetic oligonucleotide probe
  source 1..24: mol_type = other DNA
ggccggcgtctattagtagatgcc

SEQ ID NO: 4 - DNA, 5591 bp, synthetic construct
  misc_feature 1..5591: note = Recombinant plasmid pAPSE10189; beta actin sense strand + coat protein
  regulatory 214..232: note = promoter - bacteriophage T7 gene 1 promoter; regulatory_class = promoter
  misc_feature 317..335: note = bacteriophage MS2 pac sequence
  misc_feature 365..372: note = restriction endonuclease AsiSI recognition site
  misc_feature 373..667: note = beta actin sense strand gene fragment
  misc_feature 667..674: note = restriction endonuclease PmeI recognition site
  misc_feature 802..820: note = bacteriophage MS2 pac sequence
  regulatory 836..883: note = terminator - bacteriophage T7 transcription terminator; regulatory_class = terminator
  regulatory 1209..1227: note = promoter - bacteriophage T7 gene 1 promoter; regulatory_class = promoter
  gene 1300..1689: note = bacteriophage MS2 cot protein gene
  gene 1300..1689: note = bacteriophage MS2 coat protein gene
  regulatory 1722..1769: note = terminator - bacteriophage T7 transcription terminator; regulatory_class = terminator
  source 1..5591: mol_type = other DNA
ttctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatgaattcagatctcgatcccgcgaaattaatacgactcactatagggagaccacaacggtttccctctagatcacaagtttgtacaaaaaagcaggctaagaaggagatatacatacgccggccattcaaacatgaggattacccatgtatttaaatacccatgtccaggcgcgctccgcgatcgcgcacgaggtttttctgtctagtgagcagtgtccaacctcaaaagacaacatgtgtgacgacgatgtagcggctcttgtcgtagacaatggatccggtatgtgcaaagccggtttcgcaggagatgacgcaccccgtgccgtcttcccctcgatcgtcggtcgcccaaggcatcaaggagtcatggtcggtatgggacaaaaggactcatacgtaggagatgaagcccaaagcaaaagaggtatcctcaccctgaaataccccatcgaacacggtatcatcaccaactgggatgagtttaaacctttcggattataacatcacatctaggcgcgcctgacgatcaaccataccagacggaccgaatacccggtctgaacgagggcggccgcggtacccaagaagtacttagagttaattaaggagttcaaacatgaggatcacccatgtcgaagctcccacaccctagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcgggcatgcatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatccagatctcgatcccgcgaaattaatacgactcactatagggagaccacaacggtttccctctagatcacaagtttgtacaaaaaagcaggctaagaaggagatatacatatggcgtctaactttacccaattcgttctggttgataacggcggtacgggtgacgttaccgtagctccgtccaacttcgccaacggtgttgcggaatggattagctctaacagccgctctcaggcctacaaagtcacgtgctccgttcgtcagtctagcgcgcagaatcgcaaatacaccatcaaagttgaagtaccgaaagtcgcaacgcagaccgtaggcggcgtagaactcccagttgcggcctggcgctcttacctcaacatggaactgactattccgatttttgcgacgaactccgactgcgaactgattgttaaggcaatgcagggcctgctgaaagacggtaatccgatcccatctgcaatcgctgctaactctggcatttactaataagcggacgcgctgccaccgctgagcaataactagcataaccccttggggcctctaaacgggtcttgaggggttttttgctgaaaggaggaactatatccggcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaa

SEQ ID NO: 5 - DNA, 5591 bp, synthetic construct
  misc_feature 1..5591: note = Recombinant plasmid pAPSE10190; beta actin antisense strand + coat protein
  regulatory 214..232: note = promoter - bacteriophage T7 gene 1 promoter; regulatory_class = promoter
  misc_feature 317..335: note = bacteriophage MS2 pac sequence
  misc_feature 365..372: note = restriction endonuclease AsiSI recognition site
  misc_feature 373..667: note = beta actin antisense strand gene fragment
  misc_feature 667..674: note = restriction endonuclease PmeI recognition site
  misc_feature 802..820: note = bacteriophage MS2 pac sequence
  regulatory 836..883: note = terminator - bacteriophage T7 transcription terminator; regulatory_class = terminator
  regulatory 1209..1227: note = promoter - bacteriophage T7 gene 1 promoter; regulatory_class = promoter
  gene 1300..1689: note = bacteriophage MS2 coat protein gene
  regulatory 1722..1769: note = terminator - bacteriophage T7 transcription terminator; regulatory_class = terminator
  source 1..5591: mol_type = other DNA
ttctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatgaattcagatctcgatcccgcgaaattaatacgactcactatagggagaccacaacggtttccctctagatcacaagtttgtacaaaaaagcaggctaagaaggagatatacatacgccggccattcaaacatgaggattacccatgtatttaaatacccatgtccaggcgcgctccgcgatcgctcatcccagttggtgatgataccgtgttcgatggggtatttcagggtgaggatacctcttttgctttgggcttcatctcctacgtatgagtccttttgtcccataccgaccatgactccttgatgccttgggcgaccgacgatcgaggggaagacggcacggggtgcgtcatctcctgcgaaaccggctttgcacataccggatccattgtctacgacaagagccgctacatcgtcgtcacacatgttgtcttttgaggttggacactgctcactagacagaaaaacctcgtgcgtttaaacctttcggattataacatcacatctaggcgcgcctgacgatcaaccataccagacggaccgaatacccggtctgaacgagggcggccgcggtacccaagaagtacttagagttaattaaggagttcaaacatgaggatcacccatgtcgaagctcccacaccctagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcgggcatgcatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatccagatctcgatcccgcgaaattaatacgactcactatagggagaccacaacggtttccctctagatcacaagtttgtacaaaaaagcaggctaagaaggagatatacatatggcgtctaactttacccaattcgttctggttgataacggcggtacgggtgacgttaccgtagctccgtccaacttcgccaacggtgttgcggaatggattagctctaacagccgctctcaggcctacaaagtcacgtgctccgttcgtcagtctagcgcgcagaatcgcaaatacaccatcaaagttgaagtaccgaaagtcgcaacgcagaccgtaggcggcgtagaactcccagttgcggcctggcgctcttacctcaacatggaactgactattccgatttttgcgacgaactccgactgcgaactgattgttaaggcaatgcagggcctgctgaaagacggtaatccgatcccatctgcaatcgctgctaactctggcatttactaataagcggacgcgctgccaccgctgagcaataactagcataaccccttggggcctctaaacgggtcttgaggggttttttgctgaaaggaggaactatatccggcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaa

SEQ ID NO: 6 - DNA, 4789 bp, synthetic construct
  misc_feature 1..4789: note = Recombinant plasmid pAPSE10274; beta actin sense strand - coat protein
  regulatory 214..232: note = promoter - bacteriophage T7 gene 1 promoter; regulatory_class = promoter
  misc_feature 317..335: note = bacteriophage MS2 pac sequence
  misc_feature 365..372: note = restriction endonuclease AsiSI recognition site
  misc_feature 373..667: note = beta actin sense strand gene fragment
  misc_feature 667..674: note = restriction endonuclease PmeI recognition site
  misc_feature 802..820: note = bacteriophage MS2 pac sequence
  regulatory 836..883: note = terminator - bacteriophage T7 transcription terminator; regulatory_class = terminator
  misc_feature 990..995: note = restriction endonuclease SphI recognition site
  source 1..4789: mol_type = other DNA
ttctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatgaattcagatctcgatcccgcgaaattaatacgactcactatagggagaccacaacggtttccctctagatcacaagtttgtacaaaaaagcaggctaagaaggagatatacatacgccggccattcaaacatgaggattacccatgtatttaaatacccatgtccaggcgcgctccgcgatcgcgcacgaggtttttctgtctagtgagcagtgtccaacctcaaaagacaacatgtgtgacgacgatgtagcggctcttgtcgtagacaatggatccggtatgtgcaaagccggtttcgcaggagatgacgcaccccgtgccgtcttcccctcgatcgtcggtcgcccaaggcatcaaggagtcatggtcggtatgggacaaaaggactcatacgtaggagatgaagcccaaagcaaaagaggtatcctcaccctgaaataccccatcgaacacggtatcatcaccaactgggatgagtttaaacctttcggattataacatcacatctaggcgcgcctgacgatcaaccataccagacggaccgaatacccggtctgaacgagggcggccgcggtacccaagaagtacttagagttaattaaggagttcaaacatgaggatcacccatgtcgaagctcccacaccctagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcgggcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaa

SEQ ID NO: 7 - DNA, 4789 bp, synthetic construct
  misc_feature 1..4789: note = Recombinant plasmid pAPSE10275; beta actin antisense strand - coat protein
  regulatory 214..232: note = promoter - bacteriophage T7 gene 1 promoter; regulatory_class = promoter
  misc_feature 317..335: note = bacteriophage MS2 pac sequence
  misc_feature 365..372: note = restriction endonuclease AsiSI recognition site
  misc_feature 373..667: note = beta actin antisense strand gene fragment
  misc_feature 667..674: note = restriction endonuclease PmeI recogntion site
  misc_feature 802..820: note = bacteriophage MS2 pac sequence
  regulatory 836..883: note = terminator - bacteriophage T7 transcription terminator; regulatory_class = terminator
  misc_feature 990..995: note = restriction endonuclease SphI recognition site
  source 1..4789: mol_type = other DNA
ttctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatgaattcagatctcgatcccgcgaaattaatacgactcactatagggagaccacaacggtttccctctagatcacaagtttgtacaaaaaagcaggctaagaaggagatatacatacgccggccattcaaacatgaggattacccatgtatttaaatacccatgtccaggcgcgctccgcgatcgctcatcccagttggtgatgataccgtgttcgatggggtatttcagggtgaggatacctcttttgctttgggcttcatctcctacgtatgagtccttttgtcccataccgaccatgactccttgatgccttgggcgaccgacgatcgaggggaagacggcacggggtgcgtcatctcctgcgaaaccggctttgcacataccggatccattgtctacgacaagagccgctacatcgtcgtcacacatgttgtcttttgaggttggacactgctcactagacagaaaaacctcgtgcgtttaaacctttcggattataacatcacatctaggcgcgcctgacgatcaaccataccagacggaccgaatacccggtctgaacgagggcggccgcggtacccaagaagtacttagagttaattaaggagttcaaacatgaggatcacccatgtcgaagctcccacaccctagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcgggcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaa

SEQ ID NO: 8 - DNA, 3864 bp, synthetic construct
  misc_feature 1..3864: note = Recombinant plasmid pAPSE10269; beta actin stem loop + coat protein
  regulatory 214..232: note = promoter - bacteriophage T7 gene 1 promoter; regulatory_class = promoter
  misc_feature 317..335: note = bacteriophage MS2 pac sequence
  misc_feature 365..372: note = restriction endonuclease AsiSI recognition site
  misc_feature 373..667: note = beta actin sense strand gene fragment
  misc_feature 667..674: note = restriction endonuclease PmeI recognition site
  misc_feature 735..753: note = bacteriophage MS2 pac sequence
  misc_feature 832..1126: note = beta actin antisense strand gene fragment
  misc_feature 1201..1219: note = bacteriophage MS2 pac sequence
  regulatory 1235..1282: note = terminator - bacteriophage T7 transcription terminator; regulatory_class = terminator
  regulatory 1608..1626: note = promoter - bacteriophage T7 gene 1 promoter; regulatory_class = promoter
  gene 1699..2088: note = bacteriophage MS2 coat protein gene
  regulatory 2121..2168: note = terminator - bacteriophage T7 transcription terminator; regulatory_class = terminator
  source 1..3864: mol_type = other DNA
ttctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatgaattcagatctcgatcccgcgaaattaatacgactcactatagggagaccacaacggtttccctctagatcacaagtttgtacaaaaaagcaggctaagaaggagatatacatacgccggccattcaaacatgaggattacccatgtatttaaatacccatgtccaggcgcgctccgcgatcgcgcacgaggtttttctgtctagtgagcagtgtccaacctcaaaagacaacatgtgtgacgacgatgtagcggctcttgtcgtagacaatggatccggtatgtgcaaagccggtttcgcaggagatgacgcaccccgtgccgtcttcccctcgatcgtcggtcgcccaaggcatcaaggagtcatggtcggtatgggacaaaaggactcatacgtaggagatgaagcccaaagcaaaagaggtatcctcaccctgaaataccccatcgaacacggtatcatcaccaactgggatgagtttaaaccctctagctgctttacaaagtactggttccctttccagcgggatgctttatctaaacgcaacatgaggatcacccatgtcgccacatcgcttcctagttccgctgggatccatcgttggcggccgaagccgccattccatagtgagttctggcgcgcctcatcccagttggtgatgataccgtgttcgatggggtatttcagggtgaggatacctcttttgctttgggcttcatctcctacgtatgagtccttttgtcccataccgaccatgactccttgatgccttgggcgaccgacgatcgaggggaagacggcacggggtgcgtcatctcctgcgaaaccggctttgcacataccggatccattgtctacgacaagagccgctacatcgtcgtcacacatgttgtcttttgaggttggacactgctcactagacagaaaaacctcgtgccggaccgaatacccggtctgaacgagggcggccgcggtacccaagaagtacttagagttaattaaggagttcaaacatgaggatcacccatgtcgaagctcccacaccctagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcgggcatgcatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatccagatctcgatcccgcgaaattaatacgactcactatagggagaccacaacggtttccctctagatcacaagtttgtacaaaaaagcaggctaagaaggagatatacatatggcgtctaactttacccaattcgttctggttgataacggcggtacgggtgacgttaccgtagctccgtccaacttcgccaacggtgttgcggaatggattagctctaacagccgctctcaggcctacaaagtcacgtgctccgttcgtcagtctagcgcgcagaatcgcaaatacaccatcaaagttgaagtaccgaaagtcgcaacgcagaccgtaggcggcgtagaactcccagttgcggcctggcgctcttacctcaacatggaactgactattccgatttttgcgacgaactccgactgcgaactgattgttaaggcaatgcagggcctgctgaaagacggtaatccgatcccatctgcaatcgctgctaactctggcatttactaataagcggacgcgctgccaccgctgagcaataactagcataaccccttggggcctctaaacgggtcttgaggggttttttgctgaaaggaggaactatatccggcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgat

SEQ ID NO: 9 - DNA, 5375 bp, synthetic construct
  misc_feature 1..5375: note = Recombinant plasmid pAPSE10306; beta actin stem loop - coat protein
  regulatory 214..232: note = promoter - bacteriophage T7 gene 1 promoter; regulatory_class = promoter
  misc_feature 317..335: note = bacteriophage MS2 pac sequence
  misc_feature 365..372: note = restriction endonuclease AsiSI recognition site
  misc_feature 373..667: note = beta actin sense strand gene fragment
  misc_feature 667..674: note = restriction endonuclease PmeI recognition site
  misc_feature 735..753: note = bacteriophage MS2 pac sequence
  misc_feature 832..1126: note = beta actin antisense strand gene fragment
  misc_feature 1201..1219: note = bacteriophage MS2 pac sequence
  regulatory 1235..1282: note = terminator - bacteriophage T7 transcription terminator; regulatory_class = terminator
  misc_feature 1389..1394: note = restriction endonuclease SphI recognition site
  source 1..5375: mol_type = other DNA
ttctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatgaattcagatctcgatcccgcgaaattaatacgactcactatagggagaccacaacggtttccctctagatcacaagtttgtacaaaaaagcaggctaagaaggagatatacatacgccggccattcaaacatgaggattacccatgtatttaaatacccatgtccaggcgcgctccgcgatcgcgcacgaggtttttctgtctagtgagcagtgtccaacctcaaaagacaacatgtgtgacgacgatgtagcggctcttgtcgtagacaatggatccggtatgtgcaaagccggtttcgcaggagatgacgcaccccgtgccgtcttcccctcgatcgtcggtcgcccaaggcatcaaggagtcatggtcggtatgggacaaaaggactcatacgtaggagatgaagcccaaagcaaaagaggtatcctcaccctgaaataccccatcgaacacggtatcatcaccaactgggatgagtttaaaccctctagctgctttacaaagtactggttccctttccagcgggatgctttatctaaacgcaacatgaggatcacccatgtcgccacatcgcttcctagttccgctgggatccatcgttggcggccgaagccgccattccatagtgagttctggcgcgcctcatcccagttggtgatgataccgtgttcgatggggtatttcagggtgaggatacctcttttgctttgggcttcatctcctacgtatgagtccttttgtcccataccgaccatgactccttgatgccttgggcgaccgacgatcgaggggaagacggcacggggtgcgtcatctcctgcgaaaccggctttgcacataccggatccattgtctacgacaagagccgctacatcgtcgtcacacatgttgtcttttgaggttggacactgctcactagacagaaaaacctcgtgccggaccgaatacccggtctgaacgagggcggccgcggtacccaagaagtacttagagttaattaaggagttcaaacatgaggatcacccatgtcgaagctcccacaccctagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcgggcatgcatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaa

SEQ ID NO: 10 - DNA, 5990 bp, synthetic construct
  misc_feature 1..5990: note = Recombinant plasmid pAPSE10216; beta actin stem loop + coat protein
  regulatory 214..232: note = promoter - bacteriophage T7 gene 1 promoter; regulatory_class = promoter
  misc_feature 317..335: note = bacteriophage MS2 pac sequence
  misc_feature 365..372: note = restriction endonuclease AsiSI recognition site
  misc_feature 373..667: note = beta actin sense strand gene fragment
  misc_feature 667..674: note = restriction endonuclease PmeI recognition site
  misc_feature 832..1126: note = beta actin antisense gene fragment
  misc_feature 1201..1219: note = bacteriophage MS2 pac sequence
  regulatory 1235..1282: note = terminator - bacteriophage T7 transcription terminator; regulatory_class = terminator
  regulatory 1608..1626: note = promoter - bacteriophage T7 gene 1 promoter; regulatory_class = promoter
  gene 1699..2088: note = bacteriophage MS2 coat protein gene
  regulatory 2121..2168: note = terminator - bacteriophage T7 transcription terminator; regulatory_class = terminator
  source 1..5990: mol_type = other DNA
ttctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatgaattcagatctcgatcccgcgaaattaatacgactcactatagggagaccacaacggtttccctctagatcacaagtttgtacaaaaaagcaggctaagaaggagatatacatacgccggccattcaaacatgaggattacccatgtatttaaatacccatgtccaggcgcgctccgcgatcgcgcacgaggtttttctgtctagtgagcagtgtccaacctcaaaagacaacatgtgtgacgacgatgtagcggctcttgtcgtagacaatggatccggtatgtgcaaagccggtttcgcaggagatgacgcaccccgtgccgtcttcccctcgatcgtcggtcgcccaaggcatcaaggagtcatggtcggtatgggacaaaaggactcatacgtaggagatgaagcccaaagcaaaagaggtatcctcaccctgaaataccccatcgaacacggtatcatcaccaactgggatgagtttaaaccctctagctgctttacaaagtactggttccctttccagcgggatgctttatctaaacgcaatgagagaggtattcctcaggccacatcgcttcctagttccgctgggatccatcgttggcggccgaagccgccattccatagtgagttctggcgcgcctcatcccagttggtgatgataccgtgttcgatggggtatttcagggtgaggatacctcttttgctttgggcttcatctcctacgtatgagtccttttgtcccataccgaccatgactccttgatgccttgggcgaccgacgatcgaggggaagacggcacggggtgcgtcatctcctgcgaaaccggctttgcacataccggatccattgtctacgacaagagccgctacatcgtcgtcacacatgttgtcttttgaggttggacactgctcactagacagaaaaacctcgtgccggaccgaatacccggtctgaacgagggcggccgcggtacccaagaagtacttagagttaattaaggagttcaaacatgaggatcacccatgtcgaagctcccacaccctagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcgggcatgcatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatccagatctcgatcccgcgaaattaatacgactcactatagggagaccacaacggtttccctctagatcacaagtttgtacaaaaaagcaggctaagaaggagatatacatatggcgtctaactttacccaattcgttctggttgataacggcggtacgggtgacgttaccgtagctccgtccaacttcgccaacggtgttgcggaatggattagctctaacagccgctctcaggcctacaaagtcacgtgctccgttcgtcagtctagcgcgcagaatcgcaaatacaccatcaaagttgaagtaccgaaagtcgcaacgcagaccgtaggcggcgtagaactcccagttgcggcctggcgctcttacctcaacatggaactgactattccgatttttgcgacgaactccgactgcgaactgattgttaaggcaatgcagggcctgctgaaagacggtaatccgatcccatctgcaatcgctgctaactctggcatttactaataagcggacgcgctgccaccgctgagcaataactagcataaccccttggggcctctaaacgggtcttgaggggttttttgctgaaaggaggaactatatccggcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaa

SEQ ID NO: 11 - DNA, 5188 bp, synthetic construct
  misc_feature 1..5188: note = Recombinant plasmid pAPSE10305; beta actin stem loop - coat protein
  regulatory 214..232: note = promoter - bacteriophage T7 gene 1 promoter; regulatory_class = promoter
  misc_feature 317..335: note = bacteriophage MS2 pac sequence
  misc_feature 373..667: note = beta actin sense strand gene fragment
  misc_feature 667..674: note = restriction endonuclease PmeI recognition site
  misc_feature 832..1126: note = beta actin antisense gene fragment
  misc_feature 1201..1219: note = bacteriophage MS2 pac sequence
  regulatory 1235..1282: note = terminator - bacteriophageT7 transcription terminator; regulatory_class = terminator
  misc_feature 1389..1394: note = restriction endonuclease SphI recognition site
  source 1..5188: mol_type = other DNA
ttctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatgaattcagatctcgatcccgcgaaattaatacgactcactatagggagaccacaacggtttccctctagatcacaagtttgtacaaaaaagcaggctaagaaggagatatacatacgccggccattcaaacatgaggattacccatgtatttaaatacccatgtccaggcgcgctccgcgatcgcgcacgaggtttttctgtctagtgagcagtgtccaacctcaaaagacaacatgtgtgacgacgatgtagcggctcttgtcgtagacaatggatccggtatgtgcaaagccggtttcgcaggagatgacgcaccccgtgccgtcttcccctcgatcgtcggtcgcccaaggcatcaaggagtcatggtcggtatgggacaaaaggactcatacgtaggagatgaagcccaaagcaaaagaggtatcctcaccctgaaataccccatcgaacacggtatcatcaccaactgggatgagtttaaaccctctagctgctttacaaagtactggttccctttccagcgggatgctttatctaaacgcaatgagagaggtattcctcaggccacatcgcttcctagttccgctgggatccatcgttggcggccgaagccgccattccatagtgagttctggcgcgcctcatcccagttggtgatgataccgtgttcgatggggtatttcagggtgaggatacctcttttgctttgggcttcatctcctacgtatgagtccttttgtcccataccgaccatgactccttgatgccttgggcgaccgacgatcgaggggaagacggcacggggtgcgtcatctcctgcgaaaccggctttgcacataccggatccattgtctacgacaagagccgctacatcgtcgtcacacatgttgtcttttgaggttggacactgctcactagacagaaaaacctcgtgccggaccgaatacccggtctgaacgagggcggccgcggtacccaagaagtacttagagttaattaaggagttcaaacatgaggatcacccatgtcgaagctcccacaccctagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcgggcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaa

SEQ ID NO: 12 - DNA, 5657 bp, synthetic construct
  misc_feature 1..5657: note = Recombinant plasmid pAPSE10219; beta actin stem loop + coat protein
  regulatory 10..28: note = promoter - bacteriophage T7 gene 1 promoter; regulatory_class = promoter
  misc_feature 32..39: note = restriction endonuclease AsiSI recognition site sequence
  misc_feature 40..334: note = beta actin sense gene fragment
  misc_feature 334..341: note = restriction endonuclease PmeI recognition site
  misc_feature 497..791: note = beta actin antisense strand gene fragment
  misc_feature 868..886: note = bacteriophage MS2 pac sequence
  regulatory 902..949: note = terminator - bacteriophage T7 transcription terminator; regulatory_class = terminator
  regulatory 1275..1293: note = promoter - bacteriophage T7 gene 1 promoter; regulatory_class = promoter
  gene 1366..1755: note = bacteriophage MS2 coat protein gene
  regulatory 1788..1835: note = terminator - bacteriophage T7 transcription terminator; regulatory_class = terminator
  source 1..5657: mol_type = other DNA
ttccgaaattaatacgactcactatagggaggcgatcgcgcacgaggtttttctgtctagtgagcagtgtccaacctcaaaagacaacatgtgtgacgacgatgtagcggctcttgtcgtagacaatggatccggtatgtgcaaagccggtttcgcaggagatgacgcaccccgtgccgtcttcccctcgatcgtcggtcgcccaaggcatcaaggagtcatggtcggtatgggacaaaaggactcatacgtaggagatgaagcccaaagcaaaagaggtatcctcaccctgaaataccccatcgaacacggtatcatcaccaactgggatgagtttaaaccctctagctgctttacaaagtactggttccctttccagcgggatgctttatctaaacgcaatgagagaggtattcctcaggccacatcgcttcctagttccgctgggatccatcgttggcggccgaagccgccattccatagtgagttctggcgcgcctcatcccagttggtgatgataccgtgttcgatggggtatttcagggtgaggatacctcttttgctttgggcttcatctcctacgtatgagtccttttgtcccataccgaccatgactccttgatgccttgggcgaccgacgatcgaggggaagacggcacggggtgcgtcatctcctgcgaaaccggctttgcacataccggatccattgtctacgacaagagccgctacatcgtcgtcacacatgttgtcttttgaggttggacactgctcactagacagaaaaacctcgtgccggaccgaatacccggtctgaacgagggcggccgcggtacccaagaagtacttagagttaattaaggagttcaaacatgaggatcacccatgtcgaagctcccacaccctagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcgggcatgcatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatccagatctcgatcccgcgaaattaatacgactcactatagggagaccacaacggtttccctctagatcacaagtttgtacaaaaaagcaggctaagaaggagatatacatatggcgtctaactttacccaattcgttctggttgataacggcggtacgggtgacgttaccgtagctccgtccaacttcgccaacggtgttgcggaatggattagctctaacagccgctctcaggcctacaaagtcacgtgctccgttcgtcagtctagcgcgcagaatcgcaaatacaccatcaaagttgaagtaccgaaagtcgcaacgcagaccgtaggcggcgtagaactcccagttgcggcctggcgctcttacctcaacatggaactgactattccgatttttgcgacgaactccgactgcgaactgattgttaaggcaatgcagggcctgctgaaagacggtaatccgatcccatctgcaatcgctgctaactctggcatttactaataagcggacgcgctgccaccgctgagcaataactagcataaccccttggggcctctaaacgggtcttgaggggttttttgctgaaaggaggaactatatccggcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaa

SEQ ID NO: 13 - DNA, 4855 bp, synthetic construct
  misc_feature 1..4855: note = Recombinant plasmid pAPSE10304; beta actin stem loop - coat protein
  regulatory 10..28: note = promoter - bacteriophage T7 gene 1 promoter; regulatory_class = promoter
  misc_feature 32..39: note = restriction endonuclease AsiSI recognition site
  misc_feature 40..334: note = beta actin sense gene fragment
  misc_feature 334..341: note = restriction endonuclease PmeI recognition site
  misc_feature 497..791: note = beta actin antisense strand gene fragment
  misc_feature 868..886: note = bacteriophage MS2 pac sequence
  regulatory 902..949: note = terminator - bacteriophage T7 transcription terminator; regulatory_class = terminator
  misc_feature 1056..1061: note = restriction endonuclease SphI recognition site
  source 1..4855: mol_type = other DNA
ttccgaaattaatacgactcactatagggaggcgatcgcgcacgaggtttttctgtctagtgagcagtgtccaacctcaaaagacaacatgtgtgacgacgatgtagcggctcttgtcgtagacaatggatccggtatgtgcaaagccggtttcgcaggagatgacgcaccccgtgccgtcttcccctcgatcgtcggtcgcccaaggcatcaaggagtcatggtcggtatgggacaaaaggactcatacgtaggagatgaagcccaaagcaaaagaggtatcctcaccctgaaataccccatcgaacacggtatcatcaccaactgggatgagtttaaaccctctagctgctttacaaagtactggttccctttccagcgggatgctttatctaaacgcaatgagagaggtattcctcaggccacatcgcttcctagttccgctgggatccatcgttggcggccgaagccgccattccatagtgagttctggcgcgcctcatcccagttggtgatgataccgtgttcgatggggtatttcagggtgaggatacctcttttgctttgggcttcatctcctacgtatgagtccttttgtcccataccgaccatgactccttgatgccttgggcgaccgacgatcgaggggaagacggcacggggtgcgtcatctcctgcgaaaccggctttgcacataccggatccattgtctacgacaagagccgctacatcgtcgtcacacatgttgtcttttgaggttggacactgctcactagacagaaaaacctcgtgccggaccgaatacccggtctgaacgagggcggccgcggtacccaagaagtacttagagttaattaaggagttcaaacatgaggatcacccatgtcgaagctcccacaccctagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcgggcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaa

SEQ ID NO: 14 - DNA, 5367 bp, synthetic construct
  misc_feature 1..5367: note = Recombinant plasmid pAPSE10279; beta actin stem loop + capsid
  regulatory 38..56: note = promoter - bacteriophage T7 gene 1 promoter; regulatory_class = promoter
  misc_feature 60..67: note = restriction endonuclease AsiSI recognition site
  misc_feature 68..362: note = beta actin sense gene fragment
  misc_feature 362..369: note = restriction endonuclease PmeI recognition site
  misc_feature 527..821: note = beta actin antisense strand gene fragment
  regulatory 887..934: note = terminator - bacteriophage T7 transcription terminator; regulatory_class = terminator
  regulatory 985..1003: note = promoter - bacteriophage T7 gene 1 promoter; regulatory_class = promoter
  gene 1076..1465: note = bacteriophage MS2 coat protein gene
  regulatory 1498..1545: note = terminator - bacteriophage T7 transcription terminator; regulatory_class = terminator
  source 1..5367: mol_type = other DNA
ttcttagctccggcaagcaattaagaacttccgaaattaatacgactcactatagggaggcgatcgcgcacgaggtttttctgtctagtgagcagtgtccaacctcaaaagacaacatgtgtgacgacgatgtagcggctcttgtcgtagacaatggatccggtatgtgcaaagccggtttcgcaggagatgacgcaccccgtgccgtcttcccctcgatcgtcggtcgcccaaggcatcaaggagtcatggtcggtatgggacaaaaggactcatacgtaggagatgaagcccaaagcaaaagaggtatcctcaccctgaaataccccatcgaacacggtatcatcaccaactgggatgagtttaaaccctctagctgctttacaaagtactggttccctttccagcgggatgctttatctaaacgcaatgagagaggtattcctcaggccacatcgcttcctagttccgctgggatccatcgttggcggccgaagccgccattccatagtgagttctggcgcgcctcatcccagttggtgatgataccgtgttcgatggggtatttcagggtgaggatacctcttttgctttgggcttcatctcctacgtatgagtccttttgtcccataccgaccatgactccttgatgccttgggcgaccgacgatcgaggggaagacggcacggggtgcgtcatctcctgcgaaaccggctttgcacataccggatccattgtctacgacaagagccgctacatcgtcgtcacacatgttgtcttttgaggttggacactgctcactagacagaaaaacctcgtgccggaccgaatacccggtctgaacgaggttaattaaggtacccaagaagtacttagaggcggccgcctagcataaccccttggggcctctaaacgggtcttgaggggttttttgagaaacggccgaatacacctgttcggatccagatctcgatcccgcgaaattaatacgactcactatagggagaccacaacggtttccctctagatcacaagtttgtacaaaaaagcaggctaagaaggagatatacatatggcgtctaactttacccaattcgttctggttgataacggcggtacgggtgacgttaccgtagctccgtccaacttcgccaacggtgttgcggaatggattagctctaacagccgctctcaggcctacaaagtcacgtgctccgttcgtcagtctagcgcgcagaatcgcaaatacaccatcaaagttgaagtaccgaaagtcgcaacgcagaccgtaggcggcgtagaactcccagttgcggcctggcgctcttacctcaacatggaactgactattccgatttttgcgacgaactccgactgcgaactgattgttaaggcaatgcagggcctgctgaaagacggtaatccgatcccatctgcaatcgctgctaactctggcatttactaataagcggacgcgctgccaccgctgagcaataactagcataaccccttggggcctctaaacgggtcttgaggggttttttgctgaaaggaggaactatatccggcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaa

SEQ ID NO: 15 - DNA, 4764 bp, synthetic construct
  misc_feature 1..4764: note = Recombinant plasmid pAPSE10303; beta actin stem loop - coat protein
  regulatory 38..56: note = promoter - bacteriophage T7 gene 1 promoter; regulatory_class = promoter
  misc_feature 60..67: note = restriction endonuclease AsiSI recognition site
  misc_feature 68..362: note = beta actin sense gene fragment
  misc_feature 362..369: note = restriction endonuclease PmeI recognition site
  misc_feature 527..821: note = beta actin antisense strand gene fragment
  regulatory 887..934: note = terminator - bacteriophage T7 transcription terminator; regulatory_class = terminator
  source 1..4764: mol_type = other DNA
ttcttagctccggcaagcaattaagaacttccgaaattaatacgactcactatagggaggcgatcgcgcacgaggtttttctgtctagtgagcagtgtccaacctcaaaagacaacatgtgtgacgacgatgtagcggctcttgtcgtagacaatggatccggtatgtgcaaagccggtttcgcaggagatgacgcaccccgtgccgtcttcccctcgatcgtcggtcgcccaaggcatcaaggagtcatggtcggtatgggacaaaaggactcatacgtaggagatgaagcccaaagcaaaagaggtatcctcaccctgaaataccccatcgaacacggtatcatcaccaactgggatgagtttaaaccctctagctgctttacaaagtactggttccctttccagcgggatgctttatctaaacgcaatgagagaggtattcctcaggccacatcgcttcctagttccgctgggatccatcgttggcggccgaagccgccattccatagtgagttctggcgcgcctcatcccagttggtgatgataccgtgttcgatggggtatttcagggtgaggatacctcttttgctttgggcttcatctcctacgtatgagtccttttgtcccataccgaccatgactccttgatgccttgggcgaccgacgatcgaggggaagacggcacggggtgcgtcatctcctgcgaaaccggctttgcacataccggatccattgtctacgacaagagccgctacatcgtcgtcacacatgttgtcttttgaggttggacactgctcactagacagaaaaacctcgtgccggaccgaatacccggtctgaacgaggttaattaaggtacccaagaagtacttagaggcggccgcctagcataaccccttggggcctctaaacgggtcttgaggggttttttgagaaacggccgaatacacctgttcggatccagatccaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaa

SEQ ID NO: 16 - DNA, 5940 bp, synthetic construct
  misc_feature 1..5940: note = Recombinant plasmid pAPSE10270; beta actin stem loop + coat protein
  regulatory 214..232: note = promoter - bacteriophage T7 gene 1 promoter; regulatory_class = promoter
  misc_feature 317..335: note = bacteriophage MS2 pac sequence
  misc_feature 365..372: note = restriction endonuclease AsiSI recognition site
  misc_feature 372..666: note = beta actin sense gene fragment
  misc_feature 782..1076: note = beta actin antisense gene fragment
  misc_feature 1151..1169: note = Bacteriophage MS2 pac sequence
  regulatory 1185..1232: note = terminator - bacteriophage T7 transcription terminator; regulatory_class = terminator
  regulatory 1558..1576: note = promoter - bacteriophage T7 gene 1 promoter; regulatory_class = promoter
  gene 1649..2038: note = bacteriophage MS2 coat protein gene
  regulatory 2071..2118: note = terminator - bacteriophage T7 transcription terminator; regulatory_class = terminator
  source 1..5940: mol_type = other DNA
ttctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatgaattcagatctcgatcccgcgaaattaatacgactcactatagggagaccacaacggtttccctctagatcacaagtttgtacaaaaaagcaggctaagaaggagatatacatacgccggccattcaaacatgaggattacccatgtatttaaatacccatgtccaggcgcgctccgcgatcgcgcacgaggtttttctgtctagtgagcagtgtccaacctcaaaagacaacatgtgtgacgacgatgtagcggctcttgtcgtagacaatggatccggtatgtgcaaagccggtttcgcaggagatgacgcaccccgtgccgtcttcccctcgatcgtcggtcgcccaaggcatcaaggagtcatggtcggtatgggacaaaaggactcatacgtaggagatgaagcccaaagcaaaagaggtatcctcaccctgaaataccccatcgaacacggtatcatcaccaactgggatgagtttaaaccctctagctgctttacaaagtactggttccctttccagcgggatgctttatctaaacgcaatgagagaggtattcctcaggccacatcgcttcctagttcggcgcgcctcatcccagttggtgatgataccgtgttcgatggggtatttcagggtgaggatacctcttttgctttgggcttcatctcctacgtatgagtccttttgtcccataccgaccatgactccttgatgccttgggcgaccgacgatcgaggggaagacggcacggggtgcgtcatctcctgcgaaaccggctttgcacataccggatccattgtctacgacaagagccgctacatcgtcgtcacacatgttgtcttttgaggttggacactgctcactagacagaaaaacctcgtgccggaccgaatacccggtctgaacgagggcggccgcggtacccaagaagtacttagagttaattaaggagttcaaacatgaggatcacccatgtcgaagctcccacaccctagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcgggcatgcatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatccagatctcgatcccgcgaaattaatacgactcactatagggagaccacaacggtttccctctagatcacaagtttgtacaaaaaagcaggctaagaaggagatatacatatggcgtctaactttacccaattcgttctggttgataacggcggtacgggtgacgttaccgtagctccgtccaacttcgccaacggtgttgcggaatggattagctctaacagccgctctcaggcctacaaagtcacgtgctccgttcgtcagtctagcgcgcagaatcgcaaatacaccatcaaagttgaagtaccgaaagtcgcaacgcagaccgtaggcggcgtagaactcccagttgcggcctggcgctcttacctcaacatggaactgactattccgatttttgcgacgaactccgactgcgaactgattgttaaggcaatgcagggcctgctgaaagacggtaatccgatcccatctgcaatcgctgctaactctggcatttactaataagcggacgcgctgccaccgctgagcaataactagcataaccccttggggcctctaaacgggtcttgaggggttttttgctgaaaggaggaactatatccggcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaa

SEQ ID NO: 17 - DNA, 5960 bp, synthetic construct
  misc_feature 1..5960: note = Recombinant plasmid pAPSE10271; beta actin stem loop + coat protein
  regulatory 214..232: note = promoter - bacteriophage T7 gene 1 promoter; regulatory_class = promoter
  misc_feature 317..335: note = bacteriophage MS2 pac sequence
  misc_feature 365..372: note = restriction endonuclease AsiSI recognition site
  misc_feature 372..666: note = beta actin sense gene fragment
  misc_feature 802..1096: note = beta actin antisense gene fragment
  misc_feature 1171..1189: note = bacteriophage MS2 pac sequence
  regulatory 1205..1252: note = terminator - bacteriophage T7 transcription terminator; regulatory_class = terminator
  regulatory 1578..1596: note = promoter - bacteriophage T7 gene 1 promoter; regulatory_class = promoter
  gene 1669..2058: note = bacteriophage MS2 coat protein gene
  regulatory 2091..2138: note = terminator - bacteriophage T7 transcription terminator; regulatory_class = terminator
  source 1..5960: mol_type = other DNA
ttctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatgaattcagatctcgatcccgcgaaattaatacgactcactatagggagaccacaacggtttccctctagatcacaagtttgtacaaaaaagcaggctaagaaggagatatacatacgccggccattcaaacatgaggattacccatgtatttaaatacccatgtccaggcgcgctccgcgatcgcgcacgaggtttttctgtctagtgagcagtgtccaacctcaaaagacaacatgtgtgacgacgatgtagcggctcttgtcgtagacaatggatccggtatgtgcaaagccggtttcgcaggagatgacgcaccccgtgccgtcttcccctcgatcgtcggtcgcccaaggcatcaaggagtcatggtcggtatgggacaaaaggactcatacgtaggagatgaagcccaaagcaaaagaggtatcctcaccctgaaataccccatcgaacacggtatcatcaccaactgggatgagtttaaaccctctagctgctttacaaagtactggttccctttccagcgggatgctttatctaaacgcaatgagagaggtattcctcaggccacatcgcttcctagttccgctgggatccatcgttggcggcgcgcctcatcccagttggtgatgataccgtgttcgatggggtatttcagggtgaggatacctcttttgctttgggcttcatctcctacgtatgagtccttttgtcccataccgaccatgactccttgatgccttgggcgaccgacgatcgaggggaagacggcacggggtgcgtcatctcctgcgaaaccggctttgcacataccggatccattgtctacgacaagagccgctacatcgtcgtcacacatgttgtcttttgaggttggacactgctcactagacagaaaaacctcgtgccggaccgaatacccggtctgaacgagggcggccgcggtacccaagaagtacttagagttaattaaggagttcaaacatgaggatcacccatgtcgaagctcccacaccctagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcgggcatgcatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatccagatctcgatcccgcgaaattaatacgactcactatagggagaccacaacggtttccctctagatcacaagtttgtacaaaaaagcaggctaagaaggagatatacatatggcgtctaactttacccaattcgttctggttgataacggcggtacgggtgacgttaccgtagctccgtccaacttcgccaacggtgttgcggaatggattagctctaacagccgctctcaggcctacaaagtcacgtgctccgttcgtcagtctagcgcgcagaatcgcaaatacaccatcaaagttgaagtaccgaaagtcgcaacgcagaccgtaggcggcgtagaactcccagttgcggcctggcgctcttacctcaacatggaactgactattccgatttttgcgacgaactccgactgcgaactgattgttaaggcaatgcagggcctgctgaaagacggtaatccgatcccatctgcaatcgctgctaactctggcatttactaataagcggacgcgctgccaccgctgagcaataactagcataaccccttggggcctctaaacgggtcttgaggggttttttgctgaaaggaggaactatatccggcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaa

SEQ ID NO: 18 - DNA, 5980 bp, synthetic construct
  misc_feature 1..5980: note = Recombinant plasmid pAPSE10272; beta actin stem loop + coat protein
  regulatory 214..232: note = promoter - bacteriophage T7 gene 1 promoter; regulatory_class = promoter
  misc_feature 317..335: note = misc_features - bacteriophage MS2 pac sequence
  misc_feature 365..372: note = misc_features - restriction endonuclease AsiSI recognition site
  misc_feature 372..666: note = misc_features - beta actin sense gene fragment
  misc_feature 822..1116: note = misc_features - beta actin antisense gene fragment
  misc_feature 1191..1209: note = bacteriophage MS2 pac sequence
  regulatory 1225..1272: note = terminator - bacteriophage T7 transcription terminator; regulatory_class = terminator
  regulatory 1598..1616: note = promoter - bacteriophage T7 gene 1 promoter; regulatory_class = promoter
  gene 1689..2078: note = bacteriophage MS2 coat protein gene
  regulatory 2111..2158: note = terminator - bacteriophage T7 transcription terminator; regulatory_class = terminator
  source 1..5980: mol_type = other DNA
ttctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatgaattcagatctcgatcccgcgaaattaatacgactcactatagggagaccacaacggtttccctctagatcacaagtttgtacaaaaaagcaggctaagaaggagatatacatacgccggccattcaaacatgaggattacccatgtatttaaatacccatgtccaggcgcgctccgcgatcgcgcacgaggtttttctgtctagtgagcagtgtccaacctcaaaagacaacatgtgtgacgacgatgtagcggctcttgtcgtagacaatggatccggtatgtgcaaagccggtttcgcaggagatgacgcaccccgtgccgtcttcccctcgatcgtcggtcgcccaaggcatcaaggagtcatggtcggtatgggacaaaaggactcatacgtaggagatgaagcccaaagcaaaagaggtatcctcaccctgaaataccccatcgaacacggtatcatcaccaactgggatgagtttaaaccctctagctgctttacaaagtactggttccctttccagcgggatgctttatctaaacgcaatgagagaggtattcctcaggccacatcgcttcctagttccgctgggatccatcgttggcggccgaagccgccattccatggcgcgcctcatcccagttggtgatgataccgtgttcgatggggtatttcagggtgaggatacctcttttgctttgggcttcatctcctacgtatgagtccttttgtcccataccgaccatgactccttgatgccttgggcgaccgacgatcgaggggaagacggcacggggtgcgtcatctcctgcgaaaccggctttgcacataccggatccattgtctacgacaagagccgctacatcgtcgtcacacatgttgtcttttgaggttggacactgctcactagacagaaaaacctcgtgccggaccgaatacccggtctgaacgagggcggccgcggtacccaagaagtacttagagttaattaaggagttcaaacatgaggatcacccatgtcgaagctcccacaccctagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcgggcatgcatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatccagatctcgatcccgcgaaattaatacgactcactatagggagaccacaacggtttccctctagatcacaagtttgtacaaaaaagcaggctaagaaggagatatacatatggcgtctaactttacccaattcgttctggttgataacggcggtacgggtgacgttaccgtagctccgtccaacttcgccaacggtgttgcggaatggattagctctaacagccgctctcaggcctacaaagtcacgtgctccgttcgtcagtctagcgcgcagaatcgcaaatacaccatcaaagttgaagtaccgaaagtcgcaacgcagaccgtaggcggcgtagaactcccagttgcggcctggcgctcttacctcaacatggaactgactattccgatttttgcgacgaactccgactgcgaactgattgttaaggcaatgcagggcctgctgaaagacggtaatccgatcccatctgcaatcgctgctaactctggcatttactaataagcggacgcgctgccaccgctgagcaataactagcataaccccttggggcctctaaacgggtcttgaggggttttttgctgaaaggaggaactatatccggcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaa

SEQ ID NO: 19 - DNA, 6005 bp, synthetic construct
  misc_feature 1..6005: note = Recombinant plasmid pAPSE10292; beta actin stem loop + coat protein
  regulatory 214..232: note = promoter - bacteriophage T7 gene 1 promoter; regulatory_class = promoter
  misc_feature 317..335: note = bacteriophage MS2 pac sequence
  misc_feature 365..372: note = restriction endonuclease AsiSI recognition site
  misc_feature 372..666: note = beta actin sense gene fragment
  misc_feature 797..1091: note = beta actin antisense gene fragment
  misc_feature 1166..1184: note = bacteriophage MS2 pac sequence
  regulatory 1200..1247: note = terminator - bacteriophage T7 transcription terminator; regulatory_class = terminator
  regulatory 1573..1591: note = promoter - bacteriophage T7 gene 1 promoter; regulatory_class = promoter
  gene 1664..2053: note = bacteriophage MS2 coat protein gene
  regulatory 2086..2133: note = terminator - bacteriophage T7 transcription terminator; regulatory_class = terminator
  source 1..6005: mol_type = other DNA
ttctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatgaattcagatctcgatcccgcgaaattaatacgactcactatagggagaccacaacggtttccctctagatcacaagtttgtacaaaaaagcaggctaagaaggagatatacatacgccggccattcaaacatgaggattacccatgtatttaaatacccatgtccaggcgcgctccgcgatcgcgcacgaggtttttctgtctagtgagcagtgtccaacctcaaaagacaacatgtgtgacgacgatgtagcggctcttgtcgtagacaatggatccggtatgtgcaaagccggtttcgcaggagatgacgcaccccgtgccgtcttcccctcgatcgtcggtcgcccaaggcatcaaggagtcatggtcggtatgggacaaaaggactcatacgtaggagatgaagcccaaagcaaaagaggtatcctcaccctgaaataccccatcgaacacggtatcatcaccaactgggatgagtttaaacgcaatcgcagcaaactccggcatctactaatagacgccggccattcaacatgaggattacccatgtaacctaagaagacaacaaagaagttcaactctttatgtattgatcttccggcgcgcctcatcccagttggtgatgataccgtgttcgatggggtatttcagggtgaggatacctcttttgctttgggcttcatctcctacgtatgagtccttttgtcccataccgaccatgactccttgatgccttgggcgaccgacgatcgaggggaagacggcacggggtgcgtcatctcctgcgaaaccggctttgcacataccggatccattgtctacgacaagagccgctacatcgtcgtcacacatgttgtcttttgaggttggacactgctcactagacagaaaaacctcgtgccggaccgaatacccggtctgaacgagggcggccgcggtacccaagaagtacttagagttaattaaggagttcaaacatgaggatcacccatgtcgaagctcccacaccctagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcgggcatgcatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatccagatctcgatcccgcgaaattaatacgactcactatagggagaccacaacggtttccctctagatcacaagtttgtacaaaaaagcaggctaagaaggagatatacatatggcgtctaactttacccaattcgttctggttgataacggcggtacgggtgacgttaccgtagctccgtccaacttcgccaacggtgttgcggaatggattagctctaacagccgctctcaggcctacaaagtcacgtgctccgttcgtcagtctagcgcgcagaatcgcaaatacaccatcaaagttgaagtaccgaaagtcgcaacgcagaccgtaggcggcgtagaactcccagttgcggcctggcgctcttacctcaacatggaactgactattccgatttttgcgacgaactccgactgcgaactgattgttaaggcaatgcagggcctgctgaaagacggtaatccgatcccatctgcaatcgctgctaactctggcatttactaataagcggacgcgctgccaccgctgagcaataactagcataaccccttggggcctctaaacgggtcttgaggggttttttgctgaaaggaggaactatatccggcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaacattaacctataaaaataggcgtatcacgaggccctttcgtcttcaagaa

SEQ ID NO: 20 - DNA, 5966 bp, synthetic construct
  misc_feature 1..5966: note = Recombinant plasmid pAPSE10291; beta actin stem loop + coat protein
  regulatory 214..232: note = promoter - bacteriophage T7 gene 1 promoter; regulatory_class = promoter
  misc_feature 317..335: note = bacteriophage MS2 pac sequence
  misc_feature 365..372: note = restriction endonuclease AsiSI recognition site
  misc_feature 372..666: note = beta actin sense gene fragment
  misc_feature 808..1102: note = beta actin antisense gene fragment
  misc_feature 1177..1195: note = bacteriophage MS2 pac sequence
  regulatory 1211..1258: note = terminator - bacteriophage T7 transcription terminator; regulatory_class = terminator
  regulatory 1584..1602: note = promoter - bacteriophage T7 gene 1 promoter; regulatory_class = promoter
  gene 1675..2064: note = bacteriophage MS2 coat protein gene
  regulatory 2097..2144: note = terminator - bacteriophage T7 transcription terminator; regulatory_class = terminator
  source 1..5966: mol_type = other DNA
ttctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatgaattcagatctcgatcccgcgaaattaatacgactcactatagggagaccacaacggtttccctctagatcacaagtttgtacaaaaaagcaggctaagaaggagatatacatacgccggccattcaaacatgaggattacccatgtatttaaatacccatgtccaggcgcgctccgcgatcgcgcacgaggtttttctgtctagtgagcagtgtccaacctcaaaagacaacatgtgtgacgacgatgtagcggctcttgtcgtagacaatggatccggtatgtgcaaagccggtttcgcaggagatgacgcaccccgtgccgtcttcccctcgatcgtcggtcgcccaaggcatcaaggagtcatggtcggtatgggacaaaaggactcatacgtaggagatgaagcccaaagcaaaagaggtatcctcaccctgaaataccccatcgaacacggtatcatcaccaactgggatgagtttaaacttaagcggaacaccaggcggaacgaagaggagatagagaactagattgattagaatcaaatactagaactactaaatcgaatcgatacgctaacgaaaggacctggacacgtcgacgagccgctggggcgcgcctcatcccagttggtgatgataccgtgttcgatggggtatttcagggtgaggatacctcttttgctttgggcttcatctcctacgtatgagtccttttgtcccataccgaccatgactccttgatgccttgggcgaccgacgatcgaggggaagacggcacggggtgcgtcatctcctgcgaaaccggctttgcacataccggatccattgtctacgacaagagccgctacatcgtcgtcacacatgttgtcttttgaggttggacactgctcactagacagaaaaacctcgtgccggaccgaatacccggtctgaacgagggcggccgcggtacccaagaagtacttagagttaattaaggagttcaaacatgaggatcacccatgtcgaagctcccacaccctagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcgggcatgcatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatccagatctcgatcccgcgaaattaatacgactcactatagggagaccacaacggtttccctctagatcacaagtttgtacaaaaaagcaggctaagaaggagatatacatatggcgtctaactttacccaattcgttctggttgataacggcggtacgggtgacgttaccgtagctccgtccaacttcgccaacggtgttgcggaatggattagctctaacagccgctctcaggcctacaaagtcacgtgctccgttcgtcagtctagcgcgcagaatcgcaaatacaccatcaaagttgaagtaccgaaagtcgcaacgcagaccgtaggcggcgtagaactcccagttgcggcctggcgctcttacctcaacatggaactgactattccgatttttgcgacgaactccgactgcgaactgattgttaaggcaatgcagggcctgctgaaagacggtaatccgatcccatctgcaatcgctgctaactctggcatttactaataagcggacgcgctgccaccgctgagcaataactagcataaccccttggggcctctaaacgggtcttgaggggttttttgctgaaaggaggaactatatccggcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaa

SEQ ID NO: 21 - DNA, 5502 bp, synthetic construct
  misc_feature 1..5502: note = Recombinant plasmid pAPSE10276; beta actin stem loop + coat protein
  regulatory 214..232: note = promoter - bacteriophage T7 gene 1 promoter; regulatory_class = promoter
  misc_feature 317..335: note = bacteriophage MS2 pac sequence
  misc_feature 365..372: note = restriction endonuclease AsiSI recognition site
  misc_feature 372..422: note = beta actin sense gene fragment
  misc_feature 588..638: note = beta actin antisense gene fragment
  misc_feature 713..731: note = bacteriophage MS2 pac sequence
  regulatory 747..794: note = terminator - bacteriophage T7 transcription terminator; regulatory_class = terminator
  regulatory 1120..1138: note = promoter - bacteriophage T7 gene 1 promoter; regulatory_class = promoter
  gene 1211..1600: note = bacteriophage MS2 coat protein gene
  regulatory 1633..1680: note = terminator - bacteriophage T7 transcription terminator; regulatory_class = terminator
  source 1..5502: mol_type = other DNA
ttctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatgaattcagatctcgatcccgcgaaattaatacgactcactatagggagaccacaacggtttccctctagatcacaagtttgtacaaaaaagcaggctaagaaggagatatacatacgccggccattcaaacatgaggattacccatgtatttaaatacccatgtccaggcgcgctccgcgatcgcgcacgaggtttttctgtctagtgagcagtgtccaacctcaaaagacaacagtttaaaccctctagctgctttacaaagtactggttccctttccagcgggatgctttatctaaacgcaatgagagaggtattcctcaggccacatcgcttcctagttccgctgggatccatcgttggcggccgaagccgccattccatagtgagttctggcgcgcctgttgtcttttgaggttggacactgctcactagacagaaaaacctcgtgccggaccgaatacccggtctgaacgagggcggccgcggtacccaagaagtacttagagttaattaaggagttcaaacatgaggatcacccatgtcgaagctcccacaccctagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcgggcatgcatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatccagatctcgatcccgcgaaattaatacgactcactatagggagaccacaacggtttccctctagatcacaagtttgtacaaaaaagcaggctaagaaggagatatacatatggcgtctaactttacccaattcgttctggttgataacggcggtacgggtgacgttaccgtagctccgtccaacttcgccaacggtgttgcggaatggattagctctaacagccgctctcaggcctacaaagtcacgtgctccgttcgtcagtctagcgcgcagaatcgcaaatacaccatcaaagttgaagtaccgaaagtcgcaacgcagaccgtaggcggcgtagaactcccagttgcggcctggcgctcttacctcaacatggaactgactattccgatttttgcgacgaactccgactgcgaactgattgttaaggcaatgcagggcctgctgaaagacggtaatccgatcccatctgcaatcgctgctaactctggcatttactaataagcggacgcgctgccaccgctgagcaataactagcataaccccttggggcctctaaacgggtcttgaggggttttttgctgaaaggaggaactatatccggcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaa

SEQ ID NO: 22 - DNA, 5552 bp, synthetic construct
  misc_feature 1..5552: note = Recombinant plasmid pAPSE10277; beta actin stem loop + coat protein
  regulatory 214..232: note = promoter - bacteriophage T7 gene 1 promoter; regulatory_class = promoter
  misc_feature 317..335: note = bacteriophage MS2 pac sequence
  misc_feature 365..372: note = restriction endonuclease AsiSI recognition site
  misc_feature 372..447: note = beta actin sense gene fragment
  misc_feature 613..688: note = beta actin antisense gene fragment
  misc_feature 763..781: note = bacteriophage MS2 pac sequence
  regulatory 797..844: note = terminator - bacteriophage T7 transcription terminator; regulatory_class = terminator
  regulatory 1170..1188: note = promoter - bacteriophage T7 gene 1 promoter; regulatory_class = promoter
  gene 1261..1650: note = bacteriophage MS2 coat protein gene
  regulatory 1683..1730: note = terminator - bacteriophage T7 transcription terminator; regulatory_class = terminator
  source 1..5552: mol_type = other DNA
ttctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatgaattcagatctcgatcccgcgaaattaatacgactcactatagggagaccacaacggtttccctctagatcacaagtttgtacaaaaaagcaggctaagaaggagatatacatacgccggccattcaaacatgaggattacccatgtatttaaatacccatgtccaggcgcgctccgcgatcgcgcacgaggtttttctgtctagtgagcagtgtccaacctcaaaagacaacatgtgtgacgacgatgtagcggctctgtttaaaccctctagctgctttacaaagtactggttccctttccagcgggatgctttatctaaacgcaatgagagaggtattcctcaggccacatcgcttcctagttccgctgggatccatcgttggcggccgaagccgccattccatagtgagttctggcgcgccagagccgctacatcgtcgtcacacatgttgtcttttgaggttggacactgctcactagacagaaaaacctcgtgccggaccgaatacccggtctgaacgagggcggccgcggtacccaagaagtacttagagttaattaaggagttcaaacatgaggatcacccatgtcgaagctcccacaccctagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcgggcatgcatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatccagatctcgatcccgcgaaattaatacgactcactatagggagaccacaacggtttccctctagatcacaagtttgtacaaaaaagcaggctaagaaggagatatacatatggcgtctaactttacccaattcgttctggttgataacggcggtacgggtgacgttaccgtagctccgtccaacttcgccaacggtgttgcggaatggattagctctaacagccgctctcaggcctacaaagtcacgtgctccgttcgtcagtctagcgcgcagaatcgcaaatacaccatcaaagttgaagtaccgaaagtcgcaacgcagaccgtaggcggcgtagaactcccagttgcggcctggcgctcttacctcaacatggaactgactattccgatttttgcgacgaactccgactgcgaactgattgttaaggcaatgcagggcctgctgaaagacggtaatccgatcccatctgcaatcgctgctaactctggcatttactaataagcggacgcgctgccaccgctgagcaataactagcataaccccttggggcctctaaacgggtcttgaggggttttttgctgaaaggaggaactatatccggcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaa

SEQ ID NO: 23 - DNA, 4667 bp, synthetic construct
  misc_feature 1..4667: note = Recombinant plasmid pAPSE10149; coat protein
  regulatory 1896..1914: note = promoter - bacteriophage T7 gene 1 promoter; regulatory_class = promoter
  gene 1987..2376: note = bacteriophage MS2 coat protein
  regulatory 2409..2456: note = terminator - bacteriophage T7 transcription terminator; regulatory_class = terminator
  source 1..4667: mol_type = other DNA
gaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatccagatctcgatcccgcgaaattaatacgactcactatagggagaccacaacggtttccctctagatcacaagtttgtacaaaaaagcaggctaagaaggagatatacatatggcgtctaactttacccaattcgttctggttgataacggcggtacgggtgacgttaccgtagctccgtccaacttcgccaacggtgttgcggaatggattagctctaacagccgctctcaggcctacaaagtcacgtgctccgttcgtcagtctagcgcgcagaatcgcaaatacaccatcaaagttgaagtaccgaaagtcgcaacgcagaccgtaggcggcgtagaactcccagttgcggcctggcgctcttacctcaacatggaactgactattccgatttttgcgacgaactccgactgcgaactgattgttaaggcaatgcagggcctgctgaaagacggtaatccgatcccatctgcaatcgctgctaactctggcatttactaataagcggacgcgctgccaccgctgagcaataactagcataaccccttggggcctctaaacgggtcttgaggggttttttgctgaaaggaggaactatatccggcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcccctacgtgctgctgaagttgcccgcaacagagagtggaaccaaccggtgataccacgatactatgactgagagtcaacgccatgagcggcctcatttcttattctgagttacaacagtccgcaccgctgtccggtagctccttccggtgggcgcggggcatgactatcgtcgccgcacttatgactgtcttctttatcatgcaactcgtaggacaggtgccggcagcgcccaacagtcccccggccacggggcctgccaccatacccacgccgaaacaagcgccctgcaccattatgttccggatctgcatcgcaggatgctgctggctaccctgtggaacacctacatctgtattaacgaagcgctaaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacg

SEQ ID NO: 24 - DNA, 6228 bp, synthetic construct
  misc_feature 1..6228: note = Recombinant plasmid pAPSE10366; beta actin stem loop + eGFP protein
  regulatory 214..232: note = promoter - bacteriophage T7 gene 1 promoter; regulatory_class = promoter
  misc_feature 317..335: note = bacteriophage MS2 pac sequence
  misc_feature 365..372: note = restriction endonuclease AsiSI recognition site
  misc_feature 372..666: note = beta actin sense gene fragment
  misc_feature 832..1126: note = beta actin antisense gene fragment
  misc_feature 1201..1219: note = bacteriophage MS2 pac sequence
  regulatory 1235..1282: note = terminator - bacteriophage T7 transcription terminator; regulatory_class = terminator
  regulatory 1608..1626: note = promoter - bacteriophage T7 gene 1 promoter; regulatory_class = promoter
  gene 1696..2415: note = bacteriophage MS2 coat protein gene
  regulatory 2448..2495: note = terminator - bacteriophage T7 transcription terminator; regulatory_class = terminator
  source 1..6228: mol_type = other DNA
ttctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatgaattcagatctcgatcccgcgaaattaatacgactcactatagggagaccacaacggtttccctctagatcacaagtttgtacaaaaaagcaggctaagaaggagatatacatacgccggccattcaaacatgaggattacccatgtatttaaatacccatgtccaggcgcgctccgcgatcgcgcacgaggtttttctgtctagtgagcagtgtccaacctcaaaagacaacatgtgtgacgacgatgtagcggctcttgtcgtagacaatggatccggtatgtgcaaagccggtttcgcaggagatgacgcaccccgtgccgtcttcccctcgatcgtcggtcgcccaaggcatcaaggagtcatggtcggtatgggacaaaaggactcatacgtaggagatgaagcccaaagcaaaagaggtatcctcaccctgaaataccccatcgaacacggtatcatcaccaactgggatgagtttaaaccctctagctgctttacaaagtactggttccctttccagcgggatgctttatctaaacgcaatgagagaggtattcctcaggccacatcgcttcctagttccgctgggatccatcgttggcggccgaagccgccattccatagtgagttctggcgcgcctcatcccagttggtgatgataccgtgttcgatggggtatttcagggtgaggatacctcttttgctttgggcttcatctcctacgtatgagtccttttgtcccataccgaccatgactccttgatgccttgggcgaccgacgatcgaggggaagacggcacggggtgcgtcatctcctgcgaaaccggctttgcacataccggatccattgtctacgacaagagccgctacatcgtcgtcacacatgttgtcttttgaggttggacactgctcactagacagaaaaacctcgtgccggaccgaatacccggtctgaacgagggcggccgcggtacccaagaagtacttagagttaattaaggagttcaaacatgaggatcacccatgtcgaagctcccacaccctagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcgggcatgcatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatccagatctcgatcccgcgaaattaatacgactcactatagggagaccacaacggtttccctctagatcacaagtttgtacaaaaaagcaggctaagaaggagatatacatatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataagcggacgcgctgccaccgctgagcaataactagcataaccccttggggcctctaaacgggtcttgaggggttttttgctgaaaggaggaactatatccggtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaa

SEQ ID NO: 25 - DNA, 5499 bp, synthetic construct
  misc_feature 1..5499: note = Recombinant plasmid pAPSE10359; beta actin stem loop + Qbeta coat protein
  regulatory 24..42: note = promoter - bacteriophage T7 gene 1 promoter; regulatory_class = promoter
  misc_feature 46..65: note = bacteriophage Qbeta pac sequence
  misc_feature 66..73: note = restriction endonuclease AsiSI recognition site
  misc_feature 74..367: note = beta actin sense strand gene fragment
  misc_feature 534..827: note = beta actin antisense strand gene fragment
  misc_feature 872..891: note = bacteriophage Qbeta pac sequence
  regulatory 907..954: note = terminator - bacteriophage T7 transcription terminator; regulatory_class = terminator
  regulatory 1108..1126: note = promoter - bacteriophage T7 gene 1 promoter; regulatory_class = promoter
  gene 1196..1597: note = bacteriophage Qbeta coat protein
  regulatory 1630..1677: note = terminator - bacteriophage T7 transcription terminator; regulatory_class = terminator
  source 1..5499: mol_type = other DNA
ttcagatctcgatcccgcgaaattaatacgactcactatagggagatgcatgtctaagacagcatgcgatcgcgcacgaggtttttctgtctagtgagcagtgtccaacctcaaaagacaacatgtgtgacgacgatgtagcggctcttgtcgtagacaatggatccggtatgtgcaaagccggtttcgcaggagatgacgcaccccgtgccgtcttcccctcgatcgtcggtcgcccaaggcatcaaggagtcatggtcggtatgggacaaaaggactcatacgtaggagatgaagcccaaagcaaaagaggtatcctcaccctgaaataccccatcgaacacggtatcatcaccaactgggatgagtttaaaccctctagctgctttacaaagtactggttccctttccagcgggatgctttatctaaacgcaatgagagaggtattcctcaggccacatcgcttcctagttccgctgggatccatcgttggcggccgaagccgccattccatagtgagttctggcgcgcctcatcccagttggtgatgataccgtgttcgatggggtatttcagggtgaggatacctcttttgctttgggcttcatctcctacgtatgagtccttttgtcccataccgaccatgactccttgatgccttgggcgaccgacgatcgaggggaagacggcacggggtgcgtcatctcctgcgaaaccggctttgcacataccggatccattgtctacgacaagagccgctacatcgtcgtcacacatgttgtcttttgaggttggacactgctcactagacagaaaaacctcgtgccggaccgaatacccggtctgaacgagggcggccgcggagttcaaatgcatgtctaagacagcatcgaagctcccacaccctagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcgggcatgcgcctccgcctttaggggatccagatctcgatcccgcgaaattaatacgactcactatagggagaccacaacggtttccctctagatcacaagtttgtacaaaaaagcaggctaagaaggagatatacatatggcaaaattagagactgttactttaggtaacatcgggaaagatggaaaacaaactctggtcctcaatccgcgtggggtaaatcccactaacggcgttgcctcgctttcacaagcgggtgcagttcctgcgctggagaagcgtgttaccgtttcggtatctcagccttctcgcaatcgtaagaactacaaggtccaggttaagatccagaacccgaccgcttgcactgcaaacggttcttgtgacccatccgttactcgccaggcatatgctgacgtgaccttttcgttcacgcagtatagtaccgatgaggaacgagcttttgttcgtacagagcttactgctctgctcgctagtcctctgctgatcgatgctattgatcagctgaacccagcgtattaataagcggacgcgctgccaccgctgagcaataactagcataaccccttggggcctctaaacgggtcttgaggggttttttgctgaaaggaggaactatatccggcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaa

SEQ ID NO: 26 - DNA, 5413 bp, synthetic construct
  misc_feature 1..5413: note = Recombinant plasmid pAPSE10357; beta actin stem loop + U1A protein
  regulatory 24..42: note = promoter - bacteriophage T7 gene 1 promoter; regulatory_class = promoter
  misc_feature 41..66: note = U1A binding site sequence
  misc_feature 67..74: note = restriction endonuclease AsiSI recognition site
  misc_feature 75..368: note = beta actin sense strand gene fragment
  misc_feature 535..828: note = beta actin antisense strand gene fragment
  misc_feature 873..898: note = U1A binding site sequence
  regulatory 914..961: note = terminator - bacteriophage T7 transcription terminator; regulatory_class = terminator
  regulatory 1115..1133: note = promoter - bacteriophage T7 gene 1 promoter; regulatory_class = promoter
  gene 1203..1511: note = human U1A protein gene
  regulatory 1544..1591: note = terminator - bacteriophage T7 transcription terminator; regulatory_class = terminator
  source 1..5413: mol_type = other DNA
ttcagatctcgatcccgcgaaattaatacgactcactatagggtatccattgcactccggatgcccgcgatcgcgcacgaggtttttctgtctagtgagcagtgtccaacctcaaaagacaacatgtgtgacgacgatgtagcggctcttgtcgtagacaatggatccggtatgtgcaaagccggtttcgcaggagatgacgcaccccgtgccgtcttcccctcgatcgtcggtcgcccaaggcatcaaggagtcatggtcggtatgggacaaaaggactcatacgtaggagatgaagcccaaagcaaaagaggtatcctcaccctgaaataccccatcgaacacggtatcatcaccaactgggatgagtttaaaccctctagctgctttacaaagtactggttccctttccagcgggatgctttatctaaacgcaatgagagaggtattcctcaggccacatcgcttcctagttccgctgggatccatcgttggcggccgaagccgccattccatagtgagttctggcgcgcctcatcccagttggtgatgataccgtgttcgatggggtatttcagggtgaggatacctcttttgctttgggcttcatctcctacgtatgagtccttttgtcccataccgaccatgactccttgatgccttgggcgaccgacgatcgaggggaagacggcacggggtgcgtcatctcctgcgaaaccggctttgcacataccggatccattgtctacgacaagagccgctacatcgtcgtcacacatgttgtcttttgaggttggacactgctcactagacagaaaaacctcgtgccggaccgaatacccggtctgaacgagggcggccgcggagttcaagggtatccattgcactccggatgccccgaagctcccacaccctagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcgggcatgcgcctccgcctttaggggatccagatctcgatcccgcgaaattaatacgactcactatagggagaccacaacggtttccctctagatcacaagtttgtacaaaaaagcaggctaagaaggagatatacatatggcagttcccgagacccgccctaaccacactatttatatcaacaacctcaatgagaagatcaagaaggatgagctaaaaaagtccctgtacgccatcttctcccagtttggccagatcctggatatcctggtatcacggagcctgaagatgaggggccaggcctttgtcatcttcaaggaggtcagcagcgccaccaacgccctgcgctccatgcagggtttccctttctatgacaaacctatgcgtatccagtatgccaagaccgactcagatatcattgccaagatgaaaggcaccttcgtgtaataagcggacgcgctgccaccgctgagcaataactagcataaccccttggggcctctaaacgggtcttgaggggttttttgctgaaaggaggaactatatccggcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaa

SEQ ID NO: 27 - DNA, 5504 bp, synthetic construct
  misc_feature 1..5504: note = Recombinant plasmid pAPSE10372; beta actin stem loop + truncated coat protein
  regulatory 24..42: note = promoter - bacteriophage T7 gene 1 promoter; regulatory_class = promoter
  misc_feature 127..145: note = bacteriophage MS2 pac sequence
  misc_feature 175..182: note = restriction endonuclease AsiSI recognition site
  misc_feature 179..384: note = ErkA sense strand gene fragment
  misc_feature 436..454: note = bacteriophage MS2 pac sequence
  misc_feature 504..511: note = restriction endonuclease NotI recognition site
  misc_feature 505..696: note = ErkA antisense strand gene fragment
  misc_feature 692..699: note = restriction endonuclease PacI recognition site
  misc_feature 728..746: note = bacteriophage MS2 pac sequence
  regulatory 762..809: note = terminator - bacteriophage T7 transcription terminator; regulatory_class = terminator
  regulatory 1135..1153: note = promoter - bacteriophage T7 gene 1 promoter; regulatory_class = promoter
  misc_feature 1223..1429: note = bacteriophage MS2 coat protein N-terminal fragment
  regulatory 1635..1682: note = terminator - bacteriophage T7 transcription terminator; regulatory_class = terminator
  source 1..5504: mol_type = other DNA
ttcagatctcgatcccgcgaaattaatacgactcactatagggagaccacaacggtttccctctagatcacaagtttgtacaaaaaagcaggctaagaaggagatatacatacgccggccattcaaacatgaggattacccatgtaacctaaggccggtgtccaggcgcgctccgcgatcgcacgcggacaaagttcctcaatctaatgctgaagttataaggggacaaatatttgaagttggtcctaggtatattaaactcgcctatataggtgaaggagcttatggcatggttgtgtctgcggatgacacgctaacaaaccaaagagttgcaataaaaaaaatatcgccctttgaacaccaaacttattgctactacagtttaaacgcaatcgcagcaaactccggcatctactaatagacgccggccattcaacatgaggattacccatgtaacctaagaagacaacaaagaagttcaactctttatgtattgatcttccgcggccgcggtaccggggcaataagtttggtgttcaaagggcgatattttttttattgcaactctttggtttgttagcgtgtcatccgcagacacaaccatgccataagctccttcacctatataggcgagtttaatatacctaggaccaacttcaaatatttgtccccttataacttcagcattagattgaggaactatacgaaaattaattaaggagttcaaacatgaggatcacccatgtcgaagctcccacaccctagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcgggcatgcatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatccagatctcgatcccgcgaaattaatacgactcactatagggagaccacaacggtttccctctagatcacaagtttgtacaaaaaagcaggctaagaaggagatatacatatggcgtctaactttacccaattcgttctggttgataacggcggtacgggtgacgttaccgtagctccgtccaacttcgccaacggtgttgcggaatggattagctctaacagccgctctcagggtgctccgttcgtcagtctagcgcgcagaatcgcaaatacaccatcaaagttgaagtaccgaaagtcgcaacgcagaccgtaggcggcgtagaactcccagttgcggcctggcgctcttacctcaacatggaactgactattccgatttttgcgacgaactccgactgcgaactgattgttaaggcaatgcagggcctgctgaaagacggtaatccgatcccatctgcaatcgctgctaactctggcatttactaataagcggacgcgctgccaccgctgagcaataactagcataaccccttggggcctctaaacgggtcttgaggggttttttgctgaaaggaggaactatatccggcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaa

SEQ ID NO: 28 - protein, 68 aa, synthetic construct
  REGION 1..68: note = N-terminus truncated bacteriophage MS2 coat protein sequence
MASNFTQFVLVDNGGTGDVTVAPSNFANGVAEWISSNSRSQGAPFVSLARRIANTPSKLKYRKSQRRP

SEQ ID NO: 29 - DNA, 8339 bp, synthetic construct
  misc_feature 1..8339: note = Recombinant plasmid pAPSE10429; C. glutamicum rnc deletion plasmid
  gene 346..1140: note = Kanamycin resistance gene
  regulatory 1186..1631: note = promoter - sacB promoter; regulatory_class = promoter
  gene 1632..3053: note = sacB gene
  misc_feature 5504..5510: note = restriction endonuclease BamHI recognition site
  misc_feature 5511..8136: note = PCR fragment comprising 1.5 kb of chromosomal sequencedownstream of rnc gene (5511-7012) and 1.2 kb of chromosomal sequence upstream of rnc gene (7013-8136).
  misc_feature 8136..8141: note = restrcition endonuclease SalI recognition site
  source 1..8339: mol_type = other DNA
tgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgctagaggatcgatcctttttaacccatcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagtttctgaatttgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccgagcttcctgctgaacatcaaaggcaagaaaacatctgttgtcaaagacagcatccttgaacaaggacaattaacagttaacaaataaaaacgcaaaagaaaatgccgatgggtaccgagcgaaatgaccgaccaagcgacgcccaacctgccatcacgagatttcgattccaccgccgccttctatgaaaggttgggcttcggaatcgttttccgggacgccctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgctgcttccctgctgttttgtggaatatctaccgactggaaacaggcaaatgcaggaaattactgaactgaggggacaggcgagagacgatgccaaagagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagggctggatgatcctccagcgcggggatctcatgctggagttcttcgcccaccccaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaattcgagctcggtacccggggatcccaaatccagaaaagctcatactgctcccctaatcgatggcttccccagcaagggctttggaggcgaggcgttcttcctcactgacttcccacacctcgtgagcggcgtgcagtgcgagcagaatgatgaaacaacctaggatcaaatctggccatccagacgtcgtccatgcggtaattaaggccatcatgatgatggcaatgttgatcaggacgtcatttcgggcggataggaaggcagcttggccaagcgagccaccatgttgtcgcactcgagaaataatgatggcactcgcgccgttgatcacgacggcgcccagagaagcgacgatgatcggaaacacttcgggcgcttgcggtgcggaaaaccgttgaatcgctgcccacgcagcaaaagcagcaggtgcaagaatcacaatcgccataagtttgcccatcactgcgcgcctcgccaacggccatcctagggcaatgaaaatgagcaggttgatggaggtgtcttcaagaaaatcgacactgtcagccagtagagaaacggagcctgcgcttaatgcaataaagaattctacaaagaaataagcgaagttaagcagcgcgacggtgagcacagctttgcgcactttggttgcatcaaaagcttcgctcatcagctagcgccgcttctggcagtttgggcagtagtgggagccgcggttcatgaaactctcccggatgattaatgttccgcagcgtccgcacggctccccggtttgcgcataagcattcaatgacagcgcaaagtagccggagttgccattgacgttgacatagagcgcgtcgaaagaggtgccaccttgagcaagtgctttggtcatcacgtctttgccagcttgaagaagttcttccaagcgagctagggacagtcgatcggcacgttgcaatgggtgaatttttgcttgccagagcatttcatcggcatagatatttccgatgccggagacgatctcttggttaagcaggaggcgtttgatctccgatttccgagatttcaaattccgcgcaatcgcagagaaatcagcagactcatccaatacatctgtggcaatgtgagagacgcgttcgggtactccatcaactaggtcgccgagccaccaataaccgaaggtgcgttgatcgacaaaccacacttcatcgccattatctagctcgactttggctcgaaggtgtggactaattggtgcatctggttctttgatgagcatttgtccactcatcccaaggtgaaccagtagccctaaatcgggacgggtttcgccggagggtgcgtcgataagctcaagccagaggaatttgccgcgtcgcttggcagcgctgaccctaagccctgcgatgttggcctcgatttcggggccaccgccgagttgattgcgggctgcgcgcgggtgaagcactgtggcggacacgatggtgtggccgaccatatgatcttctaaaccgcggcgcaccacctcaacttcaggcagtaggaacttctccaaaccagcccagcgcggatcgaccttttcctcgtcttctacttcttcagagacaccgtcaggagctggcgtttcatcatcctggcatgcgccgtacccaagttcttcgcagacagggttaaacggcaaggtcagaccagcttcatcaatgacagactgaagcagatcaatctggtcttggttaaccattggcagctcatcttcgtcatctgctgcatcttcaccagtaacaaagtctggatcggcagcaaaaacctcagagacgtgcagcgtcttggttggggtgagttcgcggaggcagcgggagcactgtcccagaagctgcgcttcgatatctgcttcgacggccaggcctccaccgagtggaatgatctgggcttctacgataacttttccgccctcggggatcgcgatcatttccggaccaatgcgggtcgggcttggacctgattgggtgaggtgttccggaagggcacttccacgaaggagtgcggcgacatcaaaaataaatggagatttcatgaccaggaagatcctactcgtacctcatcgtgactacatacatcagatgaggatcgctagacggaaaggtgaaaaagcctaaatggtttcttggaaactagtagtctcgatcgtcgcgttcgtagcctcgttcataatcacgctcgtagtcccgctcatattcaccacgcgctgccggttgctcatcgcgcagctcgcggccagtagctccagctcctcggcgtagtgcggaacgatcagcagtgacagaacgcaacgtggtggacagcgaggtttcaaactctgccaacttggtatccacgtagtcatcgcattcattgcgcagcttgttggagtcagcgtgcgctgcatccacaatgcggtgtgcttcttccgtggagcgacgcaccacttctgcctcgctgaccaggcgatcctgctcggcctggccttcagcaaccgcgcggcgatacgcatcattgccggagttgaccaggcgctcagattcagcctgggcgcgctcggtgacgctgtttgcttctcgacgagcgtcactcacgatgcgatccgctgtgtcattagccttagcaacgacagaatgcgcatgcttgggtcgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgataagctagcttcacgc

SEQ ID NO: 30 - DNA, 3343 bp, synthetic construct
  misc_feature 1..3343: note = synthetic DNA
  misc_feature 1..301: note = pCG1 origin of replication
  gene 2269..3063: note = kanamycin resistance gene
  source 1..3343: mol_type = other DNA
ccatggtcgtcacagagctggaagcggcagcgagaattatccgcgatcgtggcgcggtgcccgcaggcatgacaaacatcgtaaatgccgcgtttcgtgtggccgtggccgcccaggacgtgtcagcgccgccaccacctgcaccgaatcggcagcagcgtcgcgcgtcgaaaaagcgcacaggcggcaagaagcgataagctgcacgaatacctgaaaaatgttgaacgccccgtgagcggtaactcacagggcgtcggctaacccccagtccaaacctgggagaaagcgctcaaaaatgactctagcggattcacgagacattgacacaccggcctggaaattttccgctgatctgttcgacacccatcccgagctcgcgctgcgatcacgtggctggacgagcgaagaccgccgcgaattcctcgctcacctgggcagagaaaatttccagggcagcaagacccgcgacttcgccagcgcttggatcaaagacccggacacgggagaaacacagccgaagttataccgagttggttcaaaatcgcttgcccggtgccagtatgttgctctgacgcacgcgcagcacgcagccgtgcttgtcctggacattgatgtgccgagccaccaggccggcgggaaaatcgagcacgtaaaccccgaggtctacgcgattttggagcgctgggcacgcctggaaaaagcgccagcttggatcggcgtgaatccactgagcgggaaatgccagctcatctggctcattgatccggtgtatgccgcagcaggcatgagcagcccgaatatgcgcctgctggctgcaacgaccgaggaaatgacccgcgttttcggcgctgaccaggctttttcacataggctgagccggtggccactgcacgtctccgacgatcccaccgcgtaccgctggcatgcccagcacaatcgcgtggatcgcctagctgatcttatggaggttgctcgcatgatctcaggcacagaaaaacctaaaaaacgctatgagcaggagttttctagcggacgggcacgtatcgaagcggcaagaaaagccactgcggaagcaaaagcacttgccacgcttgaagcaagcctgccgagcgccgctgaagcgtctggagagctgatcgacggcgtccgtgtcctctggactgctccagggcgtgccgcccgtgatgagacggcttttcgccacgctttgactgtgggataccagttaaaagcggctggtgagcgcctaaaagacaccaagatcatcgacgcctacgagcgtgcctacaccgtcgctcaggcggtcggagcagacggccgtgagcctgatctgccgccgatgcgtgaccgccagacgatggcgcgacgtgtgcgcggctacgtcgctaaaggccagccagtcgtccctgctcgtcagacagagacgcagagcagccgagggcgaaaagctctggccactatgggaagacgtggcggtaaaaaggccgcagaacgctggaaagacccaaacagtgagtacgcccgagcacagcgagaaaaactagctaagtccagtcaacgacaagctaggaaagctaaaggaaatcgcttgaccattgcaggttggtttatgactgttgagggagagactggctcgtggcgacaatcaatgaagctatgtctgaatttagcgtgtcacgtcagaccgtgaatagagcacttaagtctgcgggcattgaacttccacgaggacgccgtaaagcttcccagtaaatgtgccatctcgtaggcagaaaacggttccccccgtaggggtctctctcttggcctcctttctaggtcgggctgattgctcttgaagctctctaggggggctcacaccataggcagataacggttccccaccggctcacctcgtaagcgcacaaggactgctcccaatgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgctagaggatcgatcctttttaacccatcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaa

SEQ ID NO: 31 - DNA, 8710 bp, synthetic construct
  misc_feature 1..8710: note = Recombinant plasmid pAPSE10430; beta actin hairpin + coat protein
  regulatory 38..56: note = promoter - bacteriophage T7 gene 1 promoter; regulatory_class = promoter
  misc_feature 60..67: note = restriction endonuclease AsiSI recognition site
  misc_feature 68..362: note = beta actin sense gene fragment
  misc_feature 362..369: note = restriction endonuclease PmeI recognition site
  misc_feature 527..821: note = beta actin antisense strand gene fragment
  regulatory 887..934: note = terminator - bacteriophage T7 transcription terminator; regulatory_class = terminator
  regulatory 985..1003: note = promoter - bacteriophage T7 gene 1 promoter; regulatory_class = promoter
  gene 1076..1465: note = bacteriophage MS2 coat protein gene
  regulatory 1498..1545: note = terminator - bacteriophage T7 transcription terminator; regulatory_class = terminator
  misc_feature 1981..5324: note = synthetic DNA (SEQ ID NO: 30)
  source 1..8710: mol_type = other DNA
ttcttagctccggcaagcaattaagaacttccgaaattaatacgactcactatagggaggcgatcgcgcacgaggtttttctgtctagtgagcagtgtccaacctcaaaagacaacatgtgtgacgacgatgtagcggctcttgtcgtagacaatggatccggtatgtgcaaagccggtttcgcaggagatgacgcaccccgtgccgtcttcccctcgatcgtcggtcgcccaaggcatcaaggagtcatggtcggtatgggacaaaaggactcatacgtaggagatgaagcccaaagcaaaagaggtatcctcaccctgaaataccccatcgaacacggtatcatcaccaactgggatgagtttaaaccctctagctgctttacaaagtactggttccctttccagcgggatgctttatctaaacgcaatgagagaggtattcctcaggccacatcgcttcctagttccgctgggatccatcgttggcggccgaagccgccattccatagtgagttctggcgcgcctcatcccagttggtgatgataccgtgttcgatggggtatttcagggtgaggatacctcttttgctttgggcttcatctcctacgtatgagtccttttgtcccataccgaccatgactccttgatgccttgggcgaccgacgatcgaggggaagacggcacggggtgcgtcatctcctgcgaaaccggctttgcacataccggatccattgtctacgacaagagccgctacatcgtcgtcacacatgttgtcttttgaggttggacactgctcactagacagaaaaacctcgtgccggaccgaatacccggtctgaacgaggttaattaaggtacccaagaagtacttagaggcggccgcctagcataaccccttggggcctctaaacgggtcttgaggggttttttgagaaacggccgaatacacctgttcggatccagatctcgatcccgcgaaattaatacgactcactatagggagaccacaacggtttccctctagatcacaagtttgtacaaaaaagcaggctaagaaggagatatacatatggcgtctaactttacccaattcgttctggttgataacggcggtacgggtgacgttaccgtagctccgtccaacttcgccaacggtgttgcggaatggattagctctaacagccgctctcaggcctacaaagtcacgtgctccgttcgtcagtctagcgcgcagaatcgcaaatacaccatcaaagttgaagtaccgaaagtcgcaacgcagaccgtaggcggcgtagaactcccagttgcggcctggcgctcttacctcaacatggaactgactattccgatttttgcgacgaactccgactgcgaactgattgttaaggcaatgcagggcctgctgaaagacggtaatccgatcccatctgcaatcgctgctaactctggcatttactaataagcggacgcgctgccaccgctgagcaataactagcataaccccttggggcctctaaacgggtcttgaggggttttttgctgaaaggaggaactatatccggcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgccatggtcgtcacagagctggaagcggcagcgagaattatccgcgatcgtggcgcggtgcccgcaggcatgacaaacatcgtaaatgccgcgtttcgtgtggccgtggccgcccaggacgtgtcagcgccgccaccacctgcaccgaatcggcagcagcgtcgcgcgtcgaaaaagcgcacaggcggcaagaagcgataagctgcacgaatacctgaaaaatgttgaacgccccgtgagcggtaactcacagggcgtcggctaacccccagtccaaacctgggagaaagcgctcaaaaatgactctagcggattcacgagacattgacacaccggcctggaaattttccgctgatctgttcgacacccatcccgagctcgcgctgcgatcacgtggctggacgagcgaagaccgccgcgaattcctcgctcacctgggcagagaaaatttccagggcagcaagacccgcgacttcgccagcgcttggatcaaagacccggacacgggagaaacacagccgaagttataccgagttggttcaaaatcgcttgcccggtgccagtatgttgctctgacgcacgcgcagcacgcagccgtgcttgtcctggacattgatgtgccgagccaccaggccggcgggaaaatcgagcacgtaaaccccgaggtctacgcgattttggagcgctgggcacgcctggaaaaagcgccagcttggatcggcgtgaatccactgagcgggaaatgccagctcatctggctcattgatccggtgtatgccgcagcaggcatgagcagcccgaatatgcgcctgctggctgcaacgaccgaggaaatgacccgcgttttcggcgctgaccaggctttttcacataggctgagccggtggccactgcacgtctccgacgatcccaccgcgtaccgctggcatgcccagcacaatcgcgtggatcgcctagctgatcttatggaggttgctcgcatgatctcaggcacagaaaaacctaaaaaacgctatgagcaggagttttctagcggacgggcacgtatcgaagcggcaagaaaagccactgcggaagcaaaagcacttgccacgcttgaagcaagcctgccgagcgccgctgaagcgtctggagagctgatcgacggcgtccgtgtcctctggactgctccagggcgtgccgcccgtgatgagacggcttttcgccacgctttgactgtgggataccagttaaaagcggctggtgagcgcctaaaagacaccaagatcatcgacgcctacgagcgtgcctacaccgtcgctcaggcggtcggagcagacggccgtgagcctgatctgccgccgatgcgtgaccgccagacgatggcgcgacgtgtgcgcggctacgtcgctaaaggccagccagtcgtccctgctcgtcagacagagacgcagagcagccgagggcgaaaagctctggccactatgggaagacgtggcggtaaaaaggccgcagaacgctggaaagacccaaacagtgagtacgcccgagcacagcgagaaaaactagctaagtccagtcaacgacaagctaggaaagctaaaggaaatcgcttgaccattgcaggttggtttatgactgttgagggagagactggctcgtggcgacaatcaatgaagctatgtctgaatttagcgtgtcacgtcagaccgtgaatagagcacttaagtctgcgggcattgaacttccacgaggacgccgtaaagcttcccagtaaatgtgccatctcgtaggcagaaaacggttccccccgtaggggtctctctcttggcctcctttctaggtcgggctgattgctcttgaagctctctaggggggctcacaccataggcagataacggttccccaccggctcacctcgtaagcgcacaaggactgctcccaatgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgctagaggatcgatcctttttaacccatcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaacgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaa

SEQ ID NO: 32 - DNA, 8107 bp, synthetic construct
  misc_feature 1..8107: note = Recombinant plasmid pAPSE10431; beta actin stem loop - coat protein
  regulatory 38..56: note = promoter - bacteriophage T7 gene 1 promoter; regulatory_class = promoter
  misc_feature 60..67: note = restriction endonuclease AsiSI recognition site
  misc_feature 68..362: note = beta actin sense gene fragment
  misc_feature 362..369: note = restriction endonuclease PmeI recognition site
  misc_feature 527..821: note = beta actin antisense strand gene fragment
  regulatory 887..934: note = terminator - bacteriophage T7 transcription terminator; regulatory_class = terminator
  misc_feature 1378..4721: note = synthetic DNA (SEQ ID NO: 30)
  source 1..8107: mol_type = other DNA
ttcttagctccggcaagcaattaagaacttccgaaattaatacgactcactatagggaggcgatcgcgcacgaggtttttctgtctagtgagcagtgtccaacctcaaaagacaacatgtgtgacgacgatgtagcggctcttgtcgtagacaatggatccggtatgtgcaaagccggtttcgcaggagatgacgcaccccgtgccgtcttcccctcgatcgtcggtcgcccaaggcatcaaggagtcatggtcggtatgggacaaaaggactcatacgtaggagatgaagcccaaagcaaaagaggtatcctcaccctgaaataccccatcgaacacggtatcatcaccaactgggatgagtttaaaccctctagctgctttacaaagtactggttccctttccagcgggatgctttatctaaacgcaatgagagaggtattcctcaggccacatcgcttcctagttccgctgggatccatcgttggcggccgaagccgccattccatagtgagttctggcgcgcctcatcccagttggtgatgataccgtgttcgatggggtatttcagggtgaggatacctcttttgctttgggcttcatctcctacgtatgagtccttttgtcccataccgaccatgactccttgatgccttgggcgaccgacgatcgaggggaagacggcacggggtgcgtcatctcctgcgaaaccggctttgcacataccggatccattgtctacgacaagagccgctacatcgtcgtcacacatgttgtcttttgaggttggacactgctcactagacagaaaaacctcgtgccggaccgaatacccggtctgaacgaggttaattaaggtacccaagaagtacttagaggcggccgcctagcataaccccttggggcctctaaacgggtcttgaggggttttttgagaaacggccgaatacacctgttcggatccagatccaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgccatggtcgtcacagagctggaagcggcagcgagaattatccgcgatcgtggcgcggtgcccgcaggcatgacaaacatcgtaaatgccgcgtttcgtgtggccgtggccgcccaggacgtgtcagcgccgccaccacctgcaccgaatcggcagcagcgtcgcgcgtcgaaaaagcgcacaggcggcaagaagcgataagctgcacgaatacctgaaaaatgttgaacgccccgtgagcggtaactcacagggcgtcggctaacccccagtccaaacctgggagaaagcgctcaaaaatgactctagcggattcacgagacattgacacaccggcctggaaattttccgctgatctgttcgacacccatcccgagctcgcgctgcgatcacgtggctggacgagcgaagaccgccgcgaattcctcgctcacctgggcagagaaaatttccagggcagcaagacccgcgacttcgccagcgcttggatcaaagacccggacacgggagaaacacagccgaagttataccgagttggttcaaaatcgcttgcccggtgccagtatgttgctctgacgcacgcgcagcacgcagccgtgcttgtcctggacattgatgtgccgagccaccaggccggcgggaaaatcgagcacgtaaaccccgaggtctacgcgattttggagcgctgggcacgcctggaaaaagcgccagcttggatcggcgtgaatccactgagcgggaaatgccagctcatctggctcattgatccggtgtatgccgcagcaggcatgagcagcccgaatatgcgcctgctggctgcaacgaccgaggaaatgacccgcgttttcggcgctgaccaggctttttcacataggctgagccggtggccactgcacgtctccgacgatcccaccgcgtaccgctggcatgcccagcacaatcgcgtggatcgcctagctgatcttatggaggttgctcgcatgatctcaggcacagaaaaacctaaaaaacgctatgagcaggagttttctagcggacgggcacgtatcgaagcggcaagaaaagccactgcggaagcaaaagcacttgccacgcttgaagcaagcctgccgagcgccgctgaagcgtctggagagctgatcgacggcgtccgtgtcctctggactgctccagggcgtgccgcccgtgatgagacggcttttcgccacgctttgactgtgggataccagttaaaagcggctggtgagcgcctaaaagacaccaagatcatcgacgcctacgagcgtgcctacaccgtcgctcaggcggtcggagcagacggccgtgagcctgatctgccgccgatgcgtgaccgccagacgatggcgcgacgtgtgcgcggctacgtcgctaaaggccagccagtcgtccctgctcgtcagacagagacgcagagcagccgagggcgaaaagctctggccactatgggaagacgtggcggtaaaaaggccgcagaacgctggaaagacccaaacagtgagtacgcccgagcacagcgagaaaaactagctaagtccagtcaacgacaagctaggaaagctaaaggaaatcgcttgaccattgcaggttggtttatgactgttgagggagagactggctcgtggcgacaatcaatgaagctatgtctgaatttagcgtgtcacgtcagaccgtgaatagagcacttaagtctgcgggcattgaacttccacgaggacgccgtaaagcttcccagtaaatgtgccatctcgtaggcagaaaacggttccccccgtaggggtctctctcttggcctcctttctaggtcgggctgattgctcttgaagctctctaggggggctcacaccataggcagataacggttccccaccggctcacctcgtaagcgcacaaggactgctcccaatgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgctagaggatcgatcctttttaacccatcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaacgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaa

SEQ ID NO: 33 - DNA, 9000 bp, synthetic construct
  misc_feature 1..9000: note = Recombinant plasmid pAPSE10432; beta actin hairpin + coat protein
  regulatory 10..28: note = promoter - bacteriophage T7 gene 1 promoter; regulatory_class = promoter
  misc_feature 32..39: note = restriction endonuclease AsiSI recognition site
  misc_feature 41..334: note = beta actin sense gene fragment
  misc_feature 334..341: note = restriction endonuclease PmeI recognition site
  misc_feature 498..793: note = beta actin antisense strand gene fragment
  misc_feature 868..886: note = bacteriophage MS2 pac sequence
  regulatory 902..949: note = terminator - bacteriophage T7 transcription terminator; regulatory_class = terminator
  regulatory 1275..1293: note = promoter - bacteriophage T7 gene 1 promoter; regulatory_class = promoter
  gene 1366..1755: note = bacteriophage MS2 coat protein gene
  regulatory 1788..1835: note = terminator - bacteriophage T7 transcription terminator; regulatory_class = terminator
  misc_feature 2272..5614: note = synthetic DNA (SEQ ID NO: 30)
  source 1..9000: mol_type = other DNA
ttccgaaattaatacgactcactatagggaggcgatcgcgcacgaggtttttctgtctagtgagcagtgtccaacctcaaaagacaacatgtgtgacgacgatgtagcggctcttgtcgtagacaatggatccggtatgtgcaaagccggtttcgcaggagatgacgcaccccgtgccgtcttcccctcgatcgtcggtcgcccaaggcatcaaggagtcatggtcggtatgggacaaaaggactcatacgtaggagatgaagcccaaagcaaaagaggtatcctcaccctgaaataccccatcgaacacggtatcatcaccaactgggatgagtttaaaccctctagctgctttacaaagtactggttccctttccagcgggatgctttatctaaacgcaatgagagaggtattcctcaggccacatcgcttcctagttccgctgggatccatcgttggcggccgaagccgccattccatagtgagttctggcgcgcctcatcccagttggtgatgataccgtgttcgatggggtatttcagggtgaggatacctcttttgctttgggcttcatctcctacgtatgagtccttttgtcccataccgaccatgactccttgatgccttgggcgaccgacgatcgaggggaagacggcacggggtgcgtcatctcctgcgaaaccggctttgcacataccggatccattgtctacgacaagagccgctacatcgtcgtcacacatgttgtcttttgaggttggacactgctcactagacagaaaaacctcgtgccggaccgaatacccggtctgaacgagggcggccgcggtacccaagaagtacttagagttaattaaggagttcaaacatgaggatcacccatgtcgaagctcccacaccctagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcgggcatgcatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatccagatctcgatcccgcgaaattaatacgactcactatagggagaccacaacggtttccctctagatcacaagtttgtacaaaaaagcaggctaagaaggagatatacatatggcgtctaactttacccaattcgttctggttgataacggcggtacgggtgacgttaccgtagctccgtccaacttcgccaacggtgttgcggaatggattagctctaacagccgctctcaggcctacaaagtcacgtgctccgttcgtcagtctagcgcgcagaatcgcaaatacaccatcaaagttgaagtaccgaaagtcgcaacgcagaccgtaggcggcgtagaactcccagttgcggcctggcgctcttacctcaacatggaactgactattccgatttttgcgacgaactccgactgcgaactgattgttaaggcaatgcagggcctgctgaaagacggtaatccgatcccatctgcaatcgctgctaactctggcatttactaataagcggacgcgctgccaccgctgagcaataactagcataaccccttggggcctctaaacgggtcttgaggggttttttgctgaaaggaggaactatatccggcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgccatggtcgtcacagagctggaagcggcagcgagaattatccgcgatcgtggcgcggtgcccgcaggcatgacaaacatcgtaaatgccgcgtttcgtgtggccgtggccgcccaggacgtgtcagcgccgccaccacctgcaccgaatcggcagcagcgtcgcgcgtcgaaaaagcgcacaggcggcaagaagcgataagctgcacgaatacctgaaaaatgttgaacgccccgtgagcggtaactcacagggcgtcggctaacccccagtccaaacctgggagaaagcgctcaaaaatgactctagcggattcacgagacattgacacaccggcctggaaattttccgctgatctgttcgacacccatcccgagctcgcgctgcgatcacgtggctggacgagcgaagaccgccgcgaattcctcgctcacctgggcagagaaaatttccagggcagcaagacccgcgacttcgccagcgcttggatcaaagacccggacacgggagaaacacagccgaagttataccgagttggttcaaaatcgcttgcccggtgccagtatgttgctctgacgcacgcgcagcacgcagccgtgcttgtcctggacattgatgtgccgagccaccaggccggcgggaaaatcgagcacgtaaaccccgaggtctacgcgattttggagcgctgggcacgcctggaaaaagcgccagcttggatcggcgtgaatccactgagcgggaaatgccagctcatctggctcattgatccggtgtatgccgcagcaggcatgagcagcccgaatatgcgcctgctggctgcaacgaccgaggaaatgacccgcgttttcggcgctgaccaggctttttcacataggctgagccggtggccactgcacgtctccgacgatcccaccgcgtaccgctggcatgcccagcacaatcgcgtggatcgcctagctgatcttatggaggttgctcgcatgatctcaggcacagaaaaacctaaaaaacgctatgagcaggagttttctagcggacgggcacgtatcgaagcggcaagaaaagccactgcggaagcaaaagcacttgccacgcttgaagcaagcctgccgagcgccgctgaagcgtctggagagctgatcgacggcgtccgtgtcctctggactgctccagggcgtgccgcccgtgatgagacggcttttcgccacgctttgactgtgggataccagttaaaagcggctggtgagcgcctaaaagacaccaagatcatcgacgcctacgagcgtgcctacaccgtcgctcaggcggtcggagcagacggccgtgagcctgatctgccgccgatgcgtgaccgccagacgatggcgcgacgtgtgcgcggctacgtcgctaaaggccagccagtcgtccctgctcgtcagacagagacgcagagcagccgagggcgaaaagctctggccactatgggaagacgtggcggtaaaaaggccgcagaacgctggaaagacccaaacagtgagtacgcccgagcacagcgagaaaaactagctaagtccagtcaacgacaagctaggaaagctaaaggaaatcgcttgaccattgcaggttggtttatgactgttgagggagagactggctcgtggcgacaatcaatgaagctatgtctgaatttagcgtgtcacgtcagaccgtgaatagagcacttaagtctgcgggcattgaacttccacgaggacgccgtaaagcttcccagtaaatgtgccatctcgtaggcagaaaacggttccccccgtaggggtctctctcttggcctcctttctaggtcgggctgattgctcttgaagctctctaggggggctcacaccataggcagataacggttccccaccggctcacctcgtaagcgcacaaggactgctcccaatgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgctagaggatcgatcctttttaacccatcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaacgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaa

SEQ ID NO: 34 - DNA, 8198 bp, synthetic construct
  misc_feature 1..8198: note = Recombinant plasmid pAPSE10433; beta actin hairpin - coat protein
  regulatory 10..28: note = promoter - bacteriophage T7 gene 1 promoter; regulatory_class = promoter
  misc_feature 32..39: note = restriction endonuclease AsiSI recognition site
  misc_feature 41..334: note = beta actin sense gene fragment
  misc_feature 334..341: note = restriction endonuclease PmeI site
  misc_feature 498..793: note = beta actin antisense strand gene fragment
  misc_feature 868..886: note = bacteriophage MS2 pac sequence
  regulatory 902..949: note = terminator - bacteriophage T7 transcription terminator; regulatory_class = terminator
  misc_feature 1470..4812: note = bacteriophage T7 transcription terminator
  source 1..8198: mol_type = other DNA
ttccgaaattaatacgactcactatagggaggcgatcgcgcacgaggtttttctgtctagtgagcagtgtccaacctcaaaagacaacatgtgtgacgacgatgtagcggctcttgtcgtagacaatggatccggtatgtgcaaagccggtttcgcaggagatgacgcaccccgtgccgtcttcccctcgatcgtcggtcgcccaaggcatcaaggagtcatggtcggtatgggacaaaaggactcatacgtaggagatgaagcccaaagcaaaagaggtatcctcaccctgaaataccccatcgaacacggtatcatcaccaactgggatgagtttaaaccctctagctgctttacaaagtactggttccctttccagcgggatgctttatctaaacgcaatgagagaggtattcctcaggccacatcgcttcctagttccgctgggatccatcgttggcggccgaagccgccattccatagtgagttctggcgcgcctcatcccagttggtgatgataccgtgttcgatggggtatttcagggtgaggatacctcttttgctttgggcttcatctcctacgtatgagtccttttgtcccataccgaccatgactccttgatgccttgggcgaccgacgatcgaggggaagacggcacggggtgcgtcatctcctgcgaaaccggctttgcacataccggatccattgtctacgacaagagccgctacatcgtcgtcacacatgttgtcttttgaggttggacactgctcactagacagaaaaacctcgtgccggaccgaatacccggtctgaacgagggcggccgcggtacccaagaagtacttagagttaattaaggagttcaaacatgaggatcacccatgtcgaagctcccacaccctagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcgggcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgccatggtcgtcacagagctggaagcggcagcgagaattatccgcgatcgtggcgcggtgcccgcaggcatgacaaacatcgtaaatgccgcgtttcgtgtggccgtggccgcccaggacgtgtcagcgccgccaccacctgcaccgaatcggcagcagcgtcgcgcgtcgaaaaagcgcacaggcggcaagaagcgataagctgcacgaatacctgaaaaatgttgaacgccccgtgagcggtaactcacagggcgtcggctaacccccagtccaaacctgggagaaagcgctcaaaaatgactctagcggattcacgagacattgacacaccggcctggaaattttccgctgatctgttcgacacccatcccgagctcgcgctgcgatcacgtggctggacgagcgaagaccgccgcgaattcctcgctcacctgggcagagaaaatttccagggcagcaagacccgcgacttcgccagcgcttggatcaaagacccggacacgggagaaacacagccgaagttataccgagttggttcaaaatcgcttgcccggtgccagtatgttgctctgacgcacgcgcagcacgcagccgtgcttgtcctggacattgatgtgccgagccaccaggccggcgggaaaatcgagcacgtaaaccccgaggtctacgcgattttggagcgctgggcacgcctggaaaaagcgccagcttggatcggcgtgaatccactgagcgggaaatgccagctcatctggctcattgatccggtgtatgccgcagcaggcatgagcagcccgaatatgcgcctgctggctgcaacgaccgaggaaatgacccgcgttttcggcgctgaccaggctttttcacataggctgagccggtggccactgcacgtctccgacgatcccaccgcgtaccgctggcatgcccagcacaatcgcgtggatcgcctagctgatcttatggaggttgctcgcatgatctcaggcacagaaaaacctaaaaaacgctatgagcaggagttttctagcggacgggcacgtatcgaagcggcaagaaaagccactgcggaagcaaaagcacttgccacgcttgaagcaagcctgccgagcgccgctgaagcgtctggagagctgatcgacggcgtccgtgtcctctggactgctccagggcgtgccgcccgtgatgagacggcttttcgccacgctttgactgtgggataccagttaaaagcggctggtgagcgcctaaaagacaccaagatcatcgacgcctacgagcgtgcctacaccgtcgctcaggcggtcggagcagacggccgtgagcctgatctgccgccgatgcgtgaccgccagacgatggcgcgacgtgtgcgcggctacgtcgctaaaggccagccagtcgtccctgctcgtcagacagagacgcagagcagccgagggcgaaaagctctggccactatgggaagacgtggcggtaaaaaggccgcagaacgctggaaagacccaaacagtgagtacgcccgagcacagcgagaaaaactagctaagtccagtcaacgacaagctaggaaagctaaaggaaatcgcttgaccattgcaggttggtttatgactgttgagggagagactggctcgtggcgacaatcaatgaagctatgtctgaatttagcgtgtcacgtcagaccgtgaatagagcacttaagtctgcgggcattgaacttccacgaggacgccgtaaagcttcccagtaaatgtgccatctcgtaggcagaaaacggttccccccgtaggggtctctctcttggcctcctttctaggtcgggctgattgctcttgaagctctctaggggggctcacaccataggcagataacggttccccaccggctcacctcgtaagcgcacaaggactgctcccaatgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgctagaggatcgatcctttttaacccatcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaacgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaa

SEQ ID NO: 35 - DNA, 9327 bp, synthetic construct
  misc_feature 1..9327: note = Recombinant plasmid pAPSE10434; beta actin hairpin + coat protein
  regulatory 214..232: note = promoter - bacteriophage T7 gene 1 promoter; regulatory_class = promoter
  misc_feature 317..335: note = bacteriophage MS2 pac sequence
  misc_feature 365..372: note = restriction endonuclease AsiSI recognition site
  misc_feature 374..667: note = beta actin sense gene fragment
  misc_feature 667..674: note = restriction endonuclease PmeI recognition site
  misc_feature 832..1126: note = beta actin antisense strand gene fragment
  misc_feature 1201..1219: note = bacteriophage MS2 pac sequence
  regulatory 1235..1282: note = terminator - bacteriophage T7 transcription terminator; regulatory_class = terminator
  regulatory 1602..1620: note = promoter - bacteriophage T7 gene 1 promoter; regulatory_class = promoter
  gene 1693..2082: note = bacteriophage MS2 coat protein gene
  regulatory 2115..2162: note = terminator - bacteriophage T7 transcription terminator; regulatory_class = terminator
  misc_feature 2599..5941: note = synthetic DNA (SEQ ID NO: 30)
  source 1..9327: mol_type = other DNA
ttctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatgaattcagatctcgatcccgcgaaattaatacgactcactatagggagaccacaacggtttccctctagatcacaagtttgtacaaaaaagcaggctaagaaggagatatacatacgccggccattcaaacatgaggattacccatgtatttaaatacccatgtccaggcgcgctccgcgatcgcgcacgaggtttttctgtctagtgagcagtgtccaacctcaaaagacaacatgtgtgacgacgatgtagcggctcttgtcgtagacaatggatccggtatgtgcaaagccggtttcgcaggagatgacgcaccccgtgccgtcttcccctcgatcgtcggtcgcccaaggcatcaaggagtcatggtcggtatgggacaaaaggactcatacgtaggagatgaagcccaaagcaaaagaggtatcctcaccctgaaataccccatcgaacacggtatcatcaccaactgggatgagtttaaaccctctagctgctttacaaagtactggttccctttccagcgggatgctttatctaaacgcaatgagagaggtattcctcaggccacatcgcttcctagttccgctgggatccatcgttggcggccgaagccgccattccatagtgagttctggcgcgcctcatcccagttggtgatgataccgtgttcgatggggtatttcagggtgaggatacctcttttgctttgggcttcatctcctacgtatgagtccttttgtcccataccgaccatgactccttgatgccttgggcgaccgacgatcgaggggaagacggcacggggtgcgtcatctcctgcgaaaccggctttgcacataccggatccattgtctacgacaagagccgctacatcgtcgtcacacatgttgtcttttgaggttggacactgctcactagacagaaaaacctcgtgccggaccgaatacccggtctgaacgagggcggccgcggtacccaagaagtacttagagttaattaaggagttcaaacatgaggatcacccatgtcgaagctcccacaccctagcataaccccttggggcctctaaacgggtcttgaggggttttttgctgaaaggaggaactatatccgcacaggacgggtgtggtcgccatgatcgcgtagtcgatagtggctccaagtagcgaagcgagcaggactgggcggcgggcatgcatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatccagatctcgatcccgcgaaattaatacgactcactatagggagaccacaacggtttccctctagatcacaagtttgtacaaaaaagcaggctaagaaggagatatacatatggcgtctaactttacccaattcgttctggttgataacggcggtacgggtgacgttaccgtagctccgtccaacttcgccaacggtgttgcggaatggattagctctaacagccgctctcaggcctacaaagtcacgtgctccgttcgtcagtctagcgcgcagaatcgcaaatacaccatcaaagttgaagtaccgaaagtcgcaacgcagaccgtaggcggcgtagaactcccagttgcggcctggcgctcttacctcaacatggaactgactattccgatttttgcgacgaactccgactgcgaactgattgttaaggcaatgcagggcctgctgaaagacggtaatccgatcccatctgcaatcgctgctaactctggcatttactaataagcggacgcgctgccaccgctgagcaataactagcataaccccttggggcctctaaacgggtcttgaggggttttttgctgaaaggaggaactatatccggcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgccatggtcgtcacagagctggaagcggcagcgagaattatccgcgatcgtggcgcggtgcccgcaggcatgacaaacatcgtaaatgccgcgtttcgtgtggccgtggccgcccaggacgtgtcagcgccgccaccacctgcaccgaatcggcagcagcgtcgcgcgtcgaaaaagcgcacaggcggcaagaagcgataagctgcacgaatacctgaaaaatgttgaacgccccgtgagcggtaactcacagggcgtcggctaacccccagtccaaacctgggagaaagcgctcaaaaatgactctagcggattcacgagacattgacacaccggcctggaaattttccgctgatctgttcgacacccatcccgagctcgcgctgcgatcacgtggctggacgagcgaagaccgccgcgaattcctcgctcacctgggcagagaaaatttccagggcagcaagacccgcgacttcgccagcgcttggatcaaagacccggacacgggagaaacacagccgaagttataccgagttggttcaaaatcgcttgcccggtgccagtatgttgctctgacgcacgcgcagcacgcagccgtgcttgtcctggacattgatgtgccgagccaccaggccggcgggaaaatcgagcacgtaaaccccgaggtctacgcgattttggagcgctgggcacgcctggaaaaagcgccagcttggatcggcgtgaatccactgagcgggaaatgccagctcatctggctcattgatccggtgtatgccgcagcaggcatgagcagcccgaatatgcgcctgctggctgcaacgaccgaggaaatgacccgcgttttcggcgctgaccaggctttttcacataggctgagccggtggccactgcacgtctccgacgatcccaccgcgtaccgctggcatgcccagcacaatcgcgtggatcgcctagctgatcttatggaggttgctcgcatgatctcaggcacagaaaaacctaaaaaacgctatgagcaggagttttctagcggacgggcacgtatcgaagcggcaagaaaagccactgcggaagcaaaagcacttgccacgcttgaagcaagcctgccgagcgccgctgaagcgtctggagagctgatcgacggcgtccgtgtcctctggactgctccagggcgtgccgcccgtgatgagacggcttttcgccacgctttgactgtgggataccagttaaaagcggctggtgagcgcctaaaagacaccaagatcatcgacgcctacgagcgtgcctacaccgtcgctcaggcggtcggagcagacggccgtgagcctgatctgccgccgatgcgtgaccgccagacgatggcgcgacgtgtgcgcggctacgtcgctaaaggccagccagtcgtccctgctcgtcagacagagacgcagagcagccgagggcgaaaagctctggccactatgggaagacgtggcggtaaaaaggccgcagaacgctggaaagacccaaacagtgagtacgcccgagcacagcgagaaaaactagctaagtccagtcaacgacaagctaggaaagctaaaggaaatcgcttgaccattgcaggttggtttatgactgttgagggagagactggctcgtggcgacaatcaatgaagctatgtctgaatttagcgtgtcacgtcagaccgtgaatagagcacttaagtctgcgggcattgaacttccacgaggacgccgtaaagcttcccagtaaatgtgccatctcgtaggcagaaaacggttccccccgtaggggtctctctcttggcctcctttctaggtcgggctgattgctcttgaagctctctaggggggctcacaccataggcagataacggttccccaccggctcacctcgtaagcgcacaaggactgctcccaatgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgctagaggatcgatcctttttaacccatcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaacgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaa

SEQ ID NO: 36 - DNA, 8531 bp, synthetic construct
  misc_feature 1..8531: note = Recombinant plasmid pAPSE10435; beta actin hairpin - coat protein
  regulatory 214..232: note = promoter - bacteriophage T7 gene 1 promoter; regulatory_class = promoter
  misc_feature 317..335: note = bacteriophage MS2 pac sequence
  misc_feature 365..372: note = restriction endonuclease AsiSI recognition site
  misc_feature 374..667: note = beta actin sense gene fragment
  misc_feature 667..674: note = restriction endonuclease PmeI recognition site
  misc_feature 832..1126: note = beta actin antisense strand gene fragment
  misc_feature 1201..1219: note = bacteriophage MS2 pac sequence
  regulatory 1235..1282: note = terminator - bacteriophage T7 transcription terminator; regulatory_class = terminator
  misc_feature 1803..5145: note = synthetic DNA (SEQ ID NO: 30)
  source 1..8531: mol_type = other DNA
ttctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatgaattcagatctcgatcccgcgaaattaatacgactcactatagggagaccacaacggtttccctctagatcacaagtttgtacaaaaaagcaggctaagaaggagatatacatacgccggccattcaaacatgaggattacccatgtatttaaatacccatgtccaggcgcgctccgcgatcgcgcacgaggtttttctgtctagtgagcagtgtccaacctcaaaagacaacatgtgtgacgacgatgtagcggctcttgtcgtagacaatggatccggtatgtgcaaagccggtttcgcaggagatgacgcaccccgtgccgtcttcccctcgatcgtcggtcgcccaaggcatcaaggagtcatggtcggtatgggacaaaaggactcatacgtaggagatgaagcccaaagcaaaagaggtatcctcaccctgaaataccccatcgaacacggtatcatcaccaactgggatgagtttaaaccctctagctgctttacaaagtactggttccctttccagcgggatgctttatctaaacgcaatgagagaggtattcctcaggccacatcgcttcctagttccgctgggatccatcgttggcggccgaagccgccattccatagtgagttctggcgcgcctcatcccagttggtgatgataccgtgttcgatggggtatttcagggtgaggatacctcttttgctttgggcttcatctcctacgtatgagtccttttgtcccataccgaccatgactccttgatgccttgggcgaccgacgatcgaggggaagacggcacggggtgcgtcatctcctgcgaaaccggctttgcacataccggatccattgtctacgacaagagccgctacatcgtcgtcacacatgttgtcttttgaggttggacactgctcactagacagaaaaacctcgtgccggaccgaatacccggtctgaacgagggcggccgcggtacccaagaagtacttagagttaattaaggagttcaaacatgaggatcacccatgtcgaagctcccacaccctagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcgggcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgccatggtcgtcacagagctggaagcggcagcgagaattatccgcgatcgtggcgcggtgcccgcaggcatgacaaacatcgtaaatgccgcgtttcgtgtggccgtggccgcccaggacgtgtcagcgccgccaccacctgcaccgaatcggcagcagcgtcgcgcgtcgaaaaagcgcacaggcggcaagaagcgataagctgcacgaatacctgaaaaatgttgaacgccccgtgagcggtaactcacagggcgtcggctaacccccagtccaaacctgggagaaagcgctcaaaaatgactctagcggattcacgagacattgacacaccggcctggaaattttccgctgatctgttcgacacccatcccgagctcgcgctgcgatcacgtggctggacgagcgaagaccgccgcgaattcctcgctcacctgggcagagaaaatttccagggcagcaagacccgcgacttcgccagcgcttggatcaaagacccggacacgggagaaacacagccgaagttataccgagttggttcaaaatcgcttgcccggtgccagtatgttgctctgacgcacgcgcagcacgcagccgtgcttgtcctggacattgatgtgccgagccaccaggccggcgggaaaatcgagcacgtaaaccccgaggtctacgcgattttggagcgctgggcacgcctggaaaaagcgccagcttggatcggcgtgaatccactgagcgggaaatgccagctcatctggctcattgatccggtgtatgccgcagcaggcatgagcagcccgaatatgcgcctgctggctgcaacgaccgaggaaatgacccgcgttttcggcgctgaccaggctttttcacataggctgagccggtggccactgcacgtctccgacgatcccaccgcgtaccgctggcatgcccagcacaatcgcgtggatcgcctagctgatcttatggaggttgctcgcatgatctcaggcacagaaaaacctaaaaaacgctatgagcaggagttttctagcggacgggcacgtatcgaagcggcaagaaaagccactgcggaagcaaaagcacttgccacgcttgaagcaagcctgccgagcgccgctgaagcgtctggagagctgatcgacggcgtccgtgtcctctggactgctccagggcgtgccgcccgtgatgagacggcttttcgccacgctttgactgtgggataccagttaaaagcggctggtgagcgcctaaaagacaccaagatcatcgacgcctacgagcgtgcctacaccgtcgctcaggcggtcggagcagacggccgtgagcctgatctgccgccgatgcgtgaccgccagacgatggcgcgacgtgtgcgcggctacgtcgctaaaggccagccagtcgtccctgctcgtcagacagagacgcagagcagccgagggcgaaaagctctggccactatgggaagacgtggcggtaaaaaggccgcagaacgctggaaagacccaaacagtgagtacgcccgagcacagcgagaaaaactagctaagtccagtcaacgacaagctaggaaagctaaaggaaatcgcttgaccattgcaggttggtttatgactgttgagggagagactggctcgtggcgacaatcaatgaagctatgtctgaatttagcgtgtcacgtcagaccgtgaatagagcacttaagtctgcgggcattgaacttccacgaggacgccgtaaagcttcccagtaaatgtgccatctcgtaggcagaaaacggttccccccgtaggggtctctctcttggcctcctttctaggtcgggctgattgctcttgaagctctctaggggggctcacaccataggcagataacggttccccaccggctcacctcgtaagcgcacaaggactgctcccaatgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgctagaggatcgatcctttttaacccatcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaacgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaa

SEQ ID NO: 37 - DNA, 9520 bp, synthetic construct
  misc_feature 1..9520: note = Recombinant plasmid pAPSE10436; beta actin hairpin + coat protein
  regulatory 214..232: note = promoter - bacteriophage T7 gene 1 promoter; regulatory_class = promoter
  misc_feature 317..335: note = bacteriophage MS2 pac sequence
  misc_feature 365..372: note = restriction endonuclease AsiSI recognition site
  misc_feature 374..667: note = beta actin sense gene fragment
  misc_feature 665..672: note = restriction endonuclease PmeI recognition site
  misc_feature 735..753: note = bacteriophage MS2 pac sequence
  misc_feature 832..1126: note = beta actin antisense strand gene fragment
  misc_feature 1201..1219: note = bacteriophage MS2 pac sequence
  regulatory 1235..1282: note = terminator - bacteriophage T7 transcription terminator; regulatory_class = terminator
  regulatory 1608..1626: note = promoter - bacteriophage T7 gene 1 promoter; regulatory_class = promoter
  gene 1699..2088: note = bacteriophage MS2 coat protein gene
  regulatory 2121..2168: note = terminator - bacteriophage T7 transcription terminator; regulatory_class = terminator
  misc_feature 2792..6134: note = synthetic DNA (SEQ ID NO: 30)
  source 1..9520: mol_type = other DNA
ttctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatgaattcagatctcgatcccgcgaaattaatacgactcactatagggagaccacaacggtttccctctagatcacaagtttgtacaaaaaagcaggctaagaaggagatatacatacgccggccattcaaacatgaggattacccatgtatttaaatacccatgtccaggcgcgctccgcgatcgcgcacgaggtttttctgtctagtgagcagtgtccaacctcaaaagacaacatgtgtgacgacgatgtagcggctcttgtcgtagacaatggatccggtatgtgcaaagccggtttcgcaggagatgacgcaccccgtgccgtcttcccctcgatcgtcggtcgcccaaggcatcaaggagtcatggtcggtatgggacaaaaggactcatacgtaggagatgaagcccaaagcaaaagaggtatcctcaccctgaaataccccatcgaacacggtatcatcaccaactgggatgagtttaaaccctctagctgctttacaaagtactggttccctttccagcgggatgctttatctaaacgcaacatgaggatcacccatgtcgccacatcgcttcctagttccgctgggatccatcgttggcggccgaagccgccattccatagtgagttctggcgcgcctcatcccagttggtgatgataccgtgttcgatggggtatttcagggtgaggatacctcttttgctttgggcttcatctcctacgtatgagtccttttgtcccataccgaccatgactccttgatgccttgggcgaccgacgatcgaggggaagacggcacggggtgcgtcatctcctgcgaaaccggctttgcacataccggatccattgtctacgacaagagccgctacatcgtcgtcacacatgttgtcttttgaggttggacactgctcactagacagaaaaacctcgtgccggaccgaatacccggtctgaacgagggcggccgcggtacccaagaagtacttagagttaattaaggagttcaaacatgaggatcacccatgtcgaagctcccacaccctagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcgggcatgcatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatccagatctcgatcccgcgaaattaatacgactcactatagggagaccacaacggtttccctctagatcacaagtttgtacaaaaaagcaggctaagaaggagatatacatatggcgtctaactttacccaattcgttctggttgataacggcggtacgggtgacgttaccgtagctccgtccaacttcgccaacggtgttgcggaatggattagctctaacagccgctctcaggcctacaaagtcacgtgctccgttcgtcagtctagcgcgcagaatcgcaaatacaccatcaaagttgaagtaccgaaagtcgcaacgcagaccgtaggcggcgtagaactcccagttgcggcctggcgctcttacctcaacatggaactgactattccgatttttgcgacgaactccgactgcgaactgattgttaaggcaatgcagggcctgctgaaagacggtaatccgatcccatctgcaatcgctgctaactctggcatttactaataagcggacgcgctgccaccgctgagcaataactagcataaccccttggggcctctaaacgggtcttgaggggttttttgctgaaaggaggaactatatccggcatgcatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgccatggtcgtcacagagctggaagcggcagcgagaattatccgcgatcgtggcgcggtgcccgcaggcatgacaaacatcgtaaatgccgcgtttcgtgtggccgtggccgcccaggacgtgtcagcgccgccaccacctgcaccgaatcggcagcagcgtcgcgcgtcgaaaaagcgcacaggcggcaagaagcgataagctgcacgaatacctgaaaaatgttgaacgccccgtgagcggtaactcacagggcgtcggctaacccccagtccaaacctgggagaaagcgctcaaaaatgactctagcggattcacgagacattgacacaccggcctggaaattttccgctgatctgttcgacacccatcccgagctcgcgctgcgatcacgtggctggacgagcgaagaccgccgcgaattcctcgctcacctgggcagagaaaatttccagggcagcaagacccgcgacttcgccagcgcttggatcaaagacccggacacgggagaaacacagccgaagttataccgagttggttcaaaatcgcttgcccggtgccagtatgttgctctgacgcacgcgcagcacgcagccgtgcttgtcctggacattgatgtgccgagccaccaggccggcgggaaaatcgagcacgtaaaccccgaggtctacgcgattttggagcgctgggcacgcctggaaaaagcgccagcttggatcggcgtgaatccactgagcgggaaatgccagctcatctggctcattgatccggtgtatgccgcagcaggcatgagcagcccgaatatgcgcctgctggctgcaacgaccgaggaaatgacccgcgttttcggcgctgaccaggctttttcacataggctgagccggtggccactgcacgtctccgacgatcccaccgcgtaccgctggcatgcccagcacaatcgcgtggatcgcctagctgatcttatggaggttgctcgcatgatctcaggcacagaaaaacctaaaaaacgctatgagcaggagttttctagcggacgggcacgtatcgaagcggcaagaaaagccactgcggaagcaaaagcacttgccacgcttgaagcaagcctgccgagcgccgctgaagcgtctggagagctgatcgacggcgtccgtgtcctctggactgctccagggcgtgccgcccgtgatgagacggcttttcgccacgctttgactgtgggataccagttaaaagcggctggtgagcgcctaaaagacaccaagatcatcgacgcctacgagcgtgcctacaccgtcgctcaggcggtcggagcagacggccgtgagcctgatctgccgccgatgcgtgaccgccagacgatggcgcgacgtgtgcgcggctacgtcgctaaaggccagccagtcgtccctgctcgtcagacagagacgcagagcagccgagggcgaaaagctctggccactatgggaagacgtggcggtaaaaaggccgcagaacgctggaaagacccaaacagtgagtacgcccgagcacagcgagaaaaactagctaagtccagtcaacgacaagctaggaaagctaaaggaaatcgcttgaccattgcaggttggtttatgactgttgagggagagactggctcgtggcgacaatcaatgaagctatgtctgaatttagcgtgtcacgtcagaccgtgaatagagcacttaagtctgcgggcattgaacttccacgaggacgccgtaaagcttcccagtaaatgtgccatctcgtaggcagaaaacggttccccccgtaggggtctctctcttggcctcctttctaggtcgggctgattgctcttgaagctctctaggggggctcacaccataggcagataacggttccccaccggctcacctcgtaagcgcacaaggactgctcccaatgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgctagaggatcgatcctttttaacccatcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaacgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaa

SEQ ID NO: 38 - DNA, 8718 bp, synthetic construct
  misc_feature 1..8718: note = Recombinant plasmid pAPSE10437; beta actin hairpin - coat protein
  regulatory 214..232: note = promoter - bacteriophage T7 gene 1 promoter; regulatory_class = promoter
  misc_feature 317..335: note = bacteriophage MS2 pac sequence
  misc_feature 365..372: note = restriction endonuclease AsiSI recognition site
  misc_feature 374..667: note = beta actin sense gene fragment
  misc_feature 667..674: note = restriction endonuclease PmeI recognition site
  misc_feature 735..753: note = bacteriophage MS2 pac sequence
  misc_feature 832..1126: note = beta actin antisense strand gene fragment
  misc_feature 1201..1219: note = bacteriophage MS2 pac sequence
  regulatory 1235..1282: note = terminator - bacteriophage T7 transcription terminator; regulatory_class = terminator
  misc_feature 1990..5332: note = synthetic DNA (SEQ ID NO: 30)
  source 1..8718: mol_type = other DNA
ttctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatgaattcagatctcgatcccgcgaaattaatacgactcactatagggagaccacaacggtttccctctagatcacaagtttgtacaaaaaagcaggctaagaaggagatatacatacgccggccattcaaacatgaggattacccatgtatttaaatacccatgtccaggcgcgctccgcgatcgcgcacgaggtttttctgtctagtgagcagtgtccaacctcaaaagacaacatgtgtgacgacgatgtagcggctcttgtcgtagacaatggatccggtatgtgcaaagccggtttcgcaggagatgacgcaccccgtgccgtcttcccctcgatcgtcggtcgcccaaggcatcaaggagtcatggtcggtatgggacaaaaggactcatacgtaggagatgaagcccaaagcaaaagaggtatcctcaccctgaaataccccatcgaacacggtatcatcaccaactgggatgagtttaaaccctctagctgctttacaaagtactggttccctttccagcgggatgctttatctaaacgcaacatgaggatcacccatgtcgccacatcgcttcctagttccgctgggatccatcgttggcggccgaagccgccattccatagtgagttctggcgcgcctcatcccagttggtgatgataccgtgttcgatggggtatttcagggtgaggatacctcttttgctttgggcttcatctcctacgtatgagtccttttgtcccataccgaccatgactccttgatgccttgggcgaccgacgatcgaggggaagacggcacggggtgcgtcatctcctgcgaaaccggctttgcacataccggatccattgtctacgacaagagccgctacatcgtcgtcacacatgttgtcttttgaggttggacactgctcactagacagaaaaacctcgtgccggaccgaatacccggtctgaacgagggcggccgcggtacccaagaagtacttagagttaattaaggagttcaaacatgaggatcacccatgtcgaagctcccacaccctagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcgggcatgcatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgccatggtcgtcacagagctggaagcggcagcgagaattatccgcgatcgtggcgcggtgcccgcaggcatgacaaacatcgtaaatgccgcgtttcgtgtggccgtggccgcccaggacgtgtcagcgccgccaccacctgcaccgaatcggcagcagcgtcgcgcgtcgaaaaagcgcacaggcggcaagaagcgataagctgcacgaatacctgaaaaatgttgaacgccccgtgagcggtaactcacagggcgtcggctaacccccagtccaaacctgggagaaagcgctcaaaaatgactctagcggattcacgagacattgacacaccggcctggaaattttccgctgatctgttcgacacccatcccgagctcgcgctgcgatcacgtggctggacgagcgaagaccgccgcgaattcctcgctcacctgggcagagaaaatttccagggcagcaagacccgcgacttcgccagcgcttggatcaaagacccggacacgggagaaacacagccgaagttataccgagttggttcaaaatcgcttgcccggtgccagtatgttgctctgacgcacgcgcagcacgcagccgtgcttgtcctggacattgatgtgccgagccaccaggccggcgggaaaatcgagcacgtaaaccccgaggtctacgcgattttggagcgctgggcacgcctggaaaaagcgccagcttggatcggcgtgaatccactgagcgggaaatgccagctcatctggctcattgatccggtgtatgccgcagcaggcatgagcagcccgaatatgcgcctgctggctgcaacgaccgaggaaatgacccgcgttttcggcgctgaccaggctttttcacataggctgagccggtggccactgcacgtctccgacgatcccaccgcgtaccgctggcatgcccagcacaatcgcgtggatcgcctagctgatcttatggaggttgctcgcatgatctcaggcacagaaaaacctaaaaaacgctatgagcaggagttttctagcggacgggcacgtatcgaagcggcaagaaaagccactgcggaagcaaaagcacttgccacgcttgaagcaagcctgccgagcgccgctgaagcgtctggagagctgatcgacggcgtccgtgtcctctggactgctccagggcgtgccgcccgtgatgagacggcttttcgccacgctttgactgtgggataccagttaaaagcggctggtgagcgcctaaaagacaccaagatcatcgacgcctacgagcgtgcctacaccgtcgctcaggcggtcggagcagacggccgtgagcctgatctgccgccgatgcgtgaccgccagacgatggcgcgacgtgtgcgcggctacgtcgctaaaggccagccagtcgtccctgctcgtcagacagagacgcagagcagccgagggcgaaaagctctggccactatgggaagacgtggcggtaaaaaggccgcagaacgctggaaagacccaaacagtgagtacgcccgagcacagcgagaaaaactagctaagtccagtcaacgacaagctaggaaagctaaaggaaatcgcttgaccattgcaggttggtttatgactgttgagggagagactggctcgtggcgacaatcaatgaagctatgtctgaatttagcgtgtcacgtcagaccgtgaatagagcacttaagtctgcgggcattgaacttccacgaggacgccgtaaagcttcccagtaaatgtgccatctcgtaggcagaaaacggttccccccgtaggggtctctctcttggcctcctttctaggtcgggctgattgctcttgaagctctctaggggggctcacaccataggcagataacggttccccaccggctcacctcgtaagcgcacaaggactgctcccaatgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgctagaggatcgatcctttttaacccatcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaacgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaa

SEQ ID NO: 39 - DNA, 7250 bp, synthetic construct
  misc_feature 1..7250: note = Recombinant plasmid pAPSE10439; beta actin hairpin - coat protein
  regulatory 187..467: note = promoter - yeast TRP1 promoter; regulatory_class = promoter
  gene 468..1142: note = yeast TRP1 gene
  misc_feature 2082..2087: note = restriction endonuclease SacI recognition site
  misc_feature 2152..2157: note = restriction endonuclease EcoRI recognition site
  regulatory 2162..2826: note = promoter - yeast GAL1, 10 bidirectional prmoter; regulatory_class = promoter
  misc_feature 2844..2851: note = restriction endonuclease AsiSI recognition sequence
  misc_feature 2852..3146: note = beta actin sense gene fragment
  misc_feature 3146..3153: note = restriction endonuclease PmeI recognition site
  misc_feature 3311..3605: note = beta actin antisense strand gene fragment
  regulatory 3657..3846: note = terminator - yeast CYC1 terminator; regulatory_class = terminator
  misc_feature 5840..7182: note = yeast 2 micron ori
  source 1..7250: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaacgacattactatatatataatataggaagcatttaatagacagcatcgtaatatatgtgtactttgcagttatgacgccagatggcagtagtggaagatattctttattgaaaaatagcttgtcaccttacgtacaatcttgatccggagcttttctttttttgccgattaagaattaattcggtcgaaaaaagaaaaggagagggccaagagggagggcattggtgactattgagcacgtgagtatacgtgattaagcacacaaaggcagcttggagtatgtctgttattaatttcacaggtagttctggtccattggtgaaagtttgcggcttgcagagcacagaggccgcagaatgtgctctagattccgatgctgacttgctgggtattatatgtgtgcccaatagaaagagaacaattgacccggttattgcaaggaaaatttcaagtcttgtaaaagcatataaaaatagttcaggcactccgaaatacttggttggcgtgtttcgtaatcaacctaaggaggatgttttggctctggtcaatgattacggcattgatatcgtccaactgcatggagatgagtcgtggcaagaataccaagagttcctcggtttgccagttattaaaagactcgtatttccaaaagactgcaacatactactcagtgcagcttcacagaaacctcattcgtttattcccttgtttgattcagaagcaggtgggacaggtgaacttttggattggaactcgatttctgactgggttggaaggcaagagagccccgaaagcttacattttatgttagctggtggactgacgccagaaaatgttggtgatgcgcttagattaaatggcgttattggtgttgatgtaagcggaggtgtggagacaaatggtgtaaaagactctaacaaaatagcaaatttcgtcaaaaatgctaagaaataggttattactgagtagtatttatttaagtattgtttgtgcacttgcctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctgaattggagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgcttatttagaagtgtcaacaacgtatctaccaacgatttgacccttttccatcttttcgtaaatttctggcaaggtagacaagccgacaaccttgattggagacttgaccaaacctctggcgaagaattgttaattaagagctcagatcttatcgtcgtcatccttgtaatccatcgatactagtgcggccgccctttagtgagggttgaattcgaattttcaaaaattcttactttttttttggatggacgcaaagaagtttaataatcatattacatggcattaccaccatatacatatccatatacatatccatatctaatcttacttatatgttgtggaaatgtaaagagccccattatcttagcctaaaaaaaccttctctttggaactttcagtaatacgcttaactgctcattgctatattgaagtacggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatccatttaaatgcgatcgcgcacgaggtttttctgtctagtgagcagtgtccaacctcaaaagacaacatgtgtgacgacgatgtagcggctcttgtcgtagacaatggatccggtatgtgcaaagccggtttcgcaggagatgacgcaccccgtgccgtcttcccctcgatcgtcggtcgcccaaggcatcaaggagtcatggtcggtatgggacaaaaggactcatacgtaggagatgaagcccaaagcaaaagaggtatcctcaccctgaaataccccatcgaacacggtatcatcaccaactgggatgagtttaaaccctctagctgctttacaaagtactggttccctttccagcgggatgctttatctaaacgcaatgagagaggtattcctcaggccacatcgcttcctagttccgctgggatccatcgttggcggccgaagccgccattccatagtgagttctggcgcgcctcatcccagttggtgatgataccgtgttcgatggggtatttcagggtgaggatacctcttttgctttgggcttcatctcctacgtatgagtccttttgtcccataccgaccatgactccttgatgccttgggcgaccgacgatcgaggggaagacggcacggggtgcgtcatctcctgcgaaaccggctttgcacataccggatccattgtctacgacaagagccgctacatcgtcgtcacacatgttgtcttttgaggttggacactgctcactagacagaaaaacctcgtgccggaccgaatacccggtctgaacgagggcggccgcccgcgggctagctaagatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaagatccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatattaagaaaccattattatcatgacattaacctataaaaataggcgtatcacgaggccctttcgtc

SEQ ID NO: 40 - DNA, 7582 bp, synthetic construct
  misc_feature 1..7582: note = Recombinant plasmid pAPSE1440; beta actin hairpin + coat protein
  regulatory 187..467: note = promoter - yeast TRP1 promoter; regulatory_class = promoter
  gene 468..1142: note = yeast TRP1 gene
  misc_feature 2082..2087: note = restriction endonuclease SacI recognition site
  gene 2091..2480: note = bacteriophage MS2 coat protein gene
  misc_feature 2484..2489: note = restriction endonuclease EcoRI recognition site
  regulatory 2494..3158: note = promoter - yeast GAL1, 10 bidirectional promoter; regulatory_class = promoter
  misc_feature 3176..3183: note = restriction endonuclease AsiSI recognition sequence
  misc_feature 3185..3478: note = beta actin sense gene fragment
  misc_feature 3478..3485: note = restriction endonuclease PmeI recognition site
  misc_feature 3643..3937: note = beta actin antisense strand gene fragment
  regulatory 3989..4178: note = terminator - yeast CYC1 terminator; regulatory_class = terminator
  misc_feature 6172..7514: note = yeast 2 micron ori
  source 1..7582: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaacgacattactatatatataatataggaagcatttaatagacagcatcgtaatatatgtgtactttgcagttatgacgccagatggcagtagtggaagatattctttattgaaaaatagcttgtcaccttacgtacaatcttgatccggagcttttctttttttgccgattaagaattaattcggtcgaaaaaagaaaaggagagggccaagagggagggcattggtgactattgagcacgtgagtatacgtgattaagcacacaaaggcagcttggagtatgtctgttattaatttcacaggtagttctggtccattggtgaaagtttgcggcttgcagagcacagaggccgcagaatgtgctctagattccgatgctgacttgctgggtattatatgtgtgcccaatagaaagagaacaattgacccggttattgcaaggaaaatttcaagtcttgtaaaagcatataaaaatagttcaggcactccgaaatacttggttggcgtgtttcgtaatcaacctaaggaggatgttttggctctggtcaatgattacggcattgatatcgtccaactgcatggagatgagtcgtggcaagaataccaagagttcctcggtttgccagttattaaaagactcgtatttccaaaagactgcaacatactactcagtgcagcttcacagaaacctcattcgtttattcccttgtttgattcagaagcaggtgggacaggtgaacttttggattggaactcgatttctgactgggttggaaggcaagagagccccgaaagcttacattttatgttagctggtggactgacgccagaaaatgttggtgatgcgcttagattaaatggcgttattggtgttgatgtaagcggaggtgtggagacaaatggtgtaaaagactctaacaaaatagcaaatttcgtcaaaaatgctaagaaataggttattactgagtagtatttatttaagtattgtttgtgcacttgcctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctgaattggagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgcttatttagaagtgtcaacaacgtatctaccaacgatttgacccttttccatcttttcgtaaatttctggcaaggtagacaagccgacaaccttgattggagacttgaccaaacctctggcgaagaattgttaattaagagctcttattagtaaatgccagagttagcagcgattgcagatgggatcggattaccgtctttcagcaggccctgcattgccttaacaatcagttcgcagtcggagttcgtcgcaaaaatcggaatagtcagttccatgttgaggtaagagcgccaggccgcaactgggagttctacgccgcctacggtctgcgttgcgactttcggtacttcaactttgatggtgtatttgcgattctgcgcgctagactgacgaacggagcacgtgactttgtaggcctgagagcggctgttagagctaatccattccgcaacaccgttggcgaagttggacggagctacggtaacgtcacccgtaccgccgttatcaaccagaacgaattgggtaaagttagacgccatgaattcgaattttcaaaaattcttactttttttttggatggacgcaaagaagtttaataatcatattacatggcattaccaccatatacatatccatatacatatccatatctaatcttacttatatgttgtggaaatgtaaagagccccattatcttagcctaaaaaaaccttctctttggaactttcagtaatacgcttaactgctcattgctatattgaagtacggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatccatttaaatgcgatcgcgcacgaggtttttctgtctagtgagcagtgtccaacctcaaaagacaacatgtgtgacgacgatgtagcggctcttgtcgtagacaatggatccggtatgtgcaaagccggtttcgcaggagatgacgcaccccgtgccgtcttcccctcgatcgtcggtcgcccaaggcatcaaggagtcatggtcggtatgggacaaaaggactcatacgtaggagatgaagcccaaagcaaaagaggtatcctcaccctgaaataccccatcgaacacggtatcatcaccaactgggatgagtttaaaccctctagctgctttacaaagtactggttccctttccagcgggatgctttatctaaacgcaatgagagaggtattcctcaggccacatcgcttcctagttccgctgggatccatcgttggcggccgaagccgccattccatagtgagttctggcgcgcctcatcccagttggtgatgataccgtgttcgatggggtatttcagggtgaggatacctcttttgctttgggcttcatctcctacgtatgagtccttttgtcccataccgaccatgactccttgatgccttgggcgaccgacgatcgaggggaagacggcacggggtgcgtcatctcctgcgaaaccggctttgcacataccggatccattgtctacgacaagagccgctacatcgtcgtcacacatgttgtcttttgaggttggacactgctcactagacagaaaaacctcgtgccggaccgaatacccggtctgaacgagggcggccgcccgcgggctagctaagatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaagatccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatattaagaaaccattattatcatgacattaacctataaaaataggcgtatcacgaggccctttcgtc

SEQ ID NO: 41 - DNA, 7269 bp, synthetic construct
  misc_feature 1..7269: note = Recombinant plasmid pAPSE10441; beta actin stem loop - coat protein
  regulatory 187..467: note = promoter - yeast TRP1 promoter; regulatory_class = promoter
  gene 468..1142: note = yeast TRP1 gene
  misc_feature 2082..2087: note = restriction endonuclease SacI recognition site
  misc_feature 2152..2157: note = restriction endonuclease EcoRI recognition site
  regulatory 2162..2826: note = promoter - yeast GAL1, 10 bidirectional promoter; regulatory_class = promoter
  misc_feature 2844..2851: note = restriction endonuclease AsiSI recognition sequence
  misc_feature 2852..3146: note = beta actin sense gene fragment
  misc_feature 3146..3153: note = restriction endonuclease PmeI recognition site
  misc_feature 3311..3605: note = beta actin antisense strand gene fragment
  misc_feature 3641..3659: note = bacteriophage MS2 pac sequence
  regulatory 3676..3865: note = terminator - yeast CYC1 terminator; regulatory_class = terminator
  misc_feature 5859..7201: note = yeast 2 micron ori
  source 1..7269: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaacgacattactatatatataatataggaagcatttaatagacagcatcgtaatatatgtgtactttgcagttatgacgccagatggcagtagtggaagatattctttattgaaaaatagcttgtcaccttacgtacaatcttgatccggagcttttctttttttgccgattaagaattaattcggtcgaaaaaagaaaaggagagggccaagagggagggcattggtgactattgagcacgtgagtatacgtgattaagcacacaaaggcagcttggagtatgtctgttattaatttcacaggtagttctggtccattggtgaaagtttgcggcttgcagagcacagaggccgcagaatgtgctctagattccgatgctgacttgctgggtattatatgtgtgcccaatagaaagagaacaattgacccggttattgcaaggaaaatttcaagtcttgtaaaagcatataaaaatagttcaggcactccgaaatacttggttggcgtgtttcgtaatcaacctaaggaggatgttttggctctggtcaatgattacggcattgatatcgtccaactgcatggagatgagtcgtggcaagaataccaagagttcctcggtttgccagttattaaaagactcgtatttccaaaagactgcaacatactactcagtgcagcttcacagaaacctcattcgtttattcccttgtttgattcagaagcaggtgggacaggtgaacttttggattggaactcgatttctgactgggttggaaggcaagagagccccgaaagcttacattttatgttagctggtggactgacgccagaaaatgttggtgatgcgcttagattaaatggcgttattggtgttgatgtaagcggaggtgtggagacaaatggtgtaaaagactctaacaaaatagcaaatttcgtcaaaaatgctaagaaataggttattactgagtagtatttatttaagtattgtttgtgcacttgcctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctgaattggagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgcttatttagaagtgtcaacaacgtatctaccaacgatttgacccttttccatcttttcgtaaatttctggcaaggtagacaagccgacaaccttgattggagacttgaccaaacctctggcgaagaattgttaattaagagctcagatcttatcgtcgtcatccttgtaatccatcgatactagtgcggccgccctttagtgagggttgaattcgaattttcaaaaattcttactttttttttggatggacgcaaagaagtttaataatcatattacatggcattaccaccatatacatatccatatacatatccatatctaatcttacttatatgttgtggaaatgtaaagagccccattatcttagcctaaaaaaaccttctctttggaactttcagtaatacgcttaactgctcattgctatattgaagtacggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatccatttaaatgcgatcgcgcacgaggtttttctgtctagtgagcagtgtccaacctcaaaagacaacatgtgtgacgacgatgtagcggctcttgtcgtagacaatggatccggtatgtgcaaagccggtttcgcaggagatgacgcaccccgtgccgtcttcccctcgatcgtcggtcgcccaaggcatcaaggagtcatggtcggtatgggacaaaaggactcatacgtaggagatgaagcccaaagcaaaagaggtatcctcaccctgaaataccccatcgaacacggtatcatcaccaactgggatgagtttaaaccctctagctgctttacaaagtactggttccctttccagcgggatgctttatctaaacgcaatgagagaggtattcctcaggccacatcgcttcctagttccgctgggatccatcgttggcggccgaagccgccattccatagtgagttctggcgcgcctcatcccagttggtgatgataccgtgttcgatggggtatttcagggtgaggatacctcttttgctttgggcttcatctcctacgtatgagtccttttgtcccataccgaccatgactccttgatgccttgggcgaccgacgatcgaggggaagacggcacggggtgcgtcatctcctgcgaaaccggctttgcacataccggatccattgtctacgacaagagccgctacatcgtcgtcacacatgttgtcttttgaggttggacactgctcactagacagaaaaacctcgtgccggaccgaatacccggtctgaacgagggcggccgcacatgaggatcacccatgtccgcgggctagctaagatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaagatccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatattaagaaaccattattatcatgacattaacctataaaaataggcgtatcacgaggccctttcgtc

SEQ ID NO: 42 - DNA, 7601 bp, synthetic construct
  misc_feature 1..7601: note = Recombinant plasmid pAPSE10442; beta actin hairpin + coat protein
  regulatory 187..467: note = promoter - yeast TRP1 promoter; regulatory_class = promoter
  gene 468..1142: note = yeast TRP1 gene
  misc_feature 2082..2087: note = restriction endonuclease SacI recognition site
  gene 2091..2480: note = bacteriophage MS2 coat protein gene
  misc_feature 2484..2489: note = restriction endonuclease EcoRI recognition site
  regulatory 2494..3158: note = promoter - yeast GAL1, 10 bidirectional promoter; regulatory_class = promoter
  misc_feature 3176..3183: note = restriction endonuclease AsiSI recognition sequence
  misc_feature 3185..3478: note = beta actin sense gene fragment
  misc_feature 3478..3485: note = restriction endonuclease PmeI recognition site
  misc_feature 3643..3937: note = beta actin antisense strand gene fragment
  misc_feature 3973..3991: note = bacteriophage MS2 pac sequence
  regulatory 4008..4197: note = terminator - yeast CYC1 terminator; regulatory_class = terminator
  misc_feature 6191..7533: note = yeast 2 micron ori
  source 1..7601: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaacgacattactatatatataatataggaagcatttaatagacagcatcgtaatatatgtgtactttgcagttatgacgccagatggcagtagtggaagatattctttattgaaaaatagcttgtcaccttacgtacaatcttgatccggagcttttctttttttgccgattaagaattaattcggtcgaaaaaagaaaaggagagggccaagagggagggcattggtgactattgagcacgtgagtatacgtgattaagcacacaaaggcagcttggagtatgtctgttattaatttcacaggtagttctggtccattggtgaaagtttgcggcttgcagagcacagaggccgcagaatgtgctctagattccgatgctgacttgctgggtattatatgtgtgcccaatagaaagagaacaattgacccggttattgcaaggaaaatttcaagtcttgtaaaagcatataaaaatagttcaggcactccgaaatacttggttggcgtgtttcgtaatcaacctaaggaggatgttttggctctggtcaatgattacggcattgatatcgtccaactgcatggagatgagtcgtggcaagaataccaagagttcctcggtttgccagttattaaaagactcgtatttccaaaagactgcaacatactactcagtgcagcttcacagaaacctcattcgtttattcccttgtttgattcagaagcaggtgggacaggtgaacttttggattggaactcgatttctgactgggttggaaggcaagagagccccgaaagcttacattttatgttagctggtggactgacgccagaaaatgttggtgatgcgcttagattaaatggcgttattggtgttgatgtaagcggaggtgtggagacaaatggtgtaaaagactctaacaaaatagcaaatttcgtcaaaaatgctaagaaataggttattactgagtagtatttatttaagtattgtttgtgcacttgcctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctgaattggagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgcttatttagaagtgtcaacaacgtatctaccaacgatttgacccttttccatcttttcgtaaatttctggcaaggtagacaagccgacaaccttgattggagacttgaccaaacctctggcgaagaattgttaattaagagctcttattagtaaatgccagagttagcagcgattgcagatgggatcggattaccgtctttcagcaggccctgcattgccttaacaatcagttcgcagtcggagttcgtcgcaaaaatcggaatagtcagttccatgttgaggtaagagcgccaggccgcaactgggagttctacgccgcctacggtctgcgttgcgactttcggtacttcaactttgatggtgtatttgcgattctgcgcgctagactgacgaacggagcacgtgactttgtaggcctgagagcggctgttagagctaatccattccgcaacaccgttggcgaagttggacggagctacggtaacgtcacccgtaccgccgttatcaaccagaacgaattgggtaaagttagacgccatgaattcgaattttcaaaaattcttactttttttttggatggacgcaaagaagtttaataatcatattacatggcattaccaccatatacatatccatatacatatccatatctaatcttacttatatgttgtggaaatgtaaagagccccattatcttagcctaaaaaaaccttctctttggaactttcagtaatacgcttaactgctcattgctatattgaagtacggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatccatttaaatgcgatcgcgcacgaggtttttctgtctagtgagcagtgtccaacctcaaaagacaacatgtgtgacgacgatgtagcggctcttgtcgtagacaatggatccggtatgtgcaaagccggtttcgcaggagatgacgcaccccgtgccgtcttcccctcgatcgtcggtcgcccaaggcatcaaggagtcatggtcggtatgggacaaaaggactcatacgtaggagatgaagcccaaagcaaaagaggtatcctcaccctgaaataccccatcgaacacggtatcatcaccaactgggatgagtttaaaccctctagctgctttacaaagtactggttccctttccagcgggatgctttatctaaacgcaatgagagaggtattcctcaggccacatcgcttcctagttccgctgggatccatcgttggcggccgaagccgccattccatagtgagttctggcgcgcctcatcccagttggtgatgataccgtgttcgatggggtatttcagggtgaggatacctcttttgctttgggcttcatctcctacgtatgagtccttttgtcccataccgaccatgactccttgatgccttgggcgaccgacgatcgaggggaagacggcacggggtgcgtcatctcctgcgaaaccggctttgcacataccggatccattgtctacgacaagagccgctacatcgtcgtcacacatgttgtcttttgaggttggacactgctcactagacagaaaaacctcgtgccggaccgaatacccggtctgaacgagggcggccgcacatgaggatcacccatgtccgcgggctagctaagatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaagatccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatattaagaaaccattattatcatgacattaacctataaaaataggcgtatcacgaggccctttcgtc

SEQ ID NO: 43 - DNA, 7288 bp, synthetic construct
  misc_feature 1..7288: note = Recombinant plasmid pAPSE10443; beta actin stem loop - coat protein
  regulatory 187..467: note = promoter - yeast TRP1 promoter; regulatory_class = promoter
  gene 468..1142: note = yeast TRP1 gene
  misc_feature 2082..2087: note = restriction endonuclease SacI recognition site
  misc_feature 2152..2157: note = restriction endonuclease EcoRI recognition site
  regulatory 2162..2826: note = promoter - yeast GAL1, 10 bidirectional promoter; regulatory_class = promoter
  misc_feature 2844..2862: note = bacteriophage MS2 pac sequence
  misc_feature 2863..2870: note = restriction endonuclease AsiSI recognition sequence
  misc_feature 2872..3165: note = beta actin sense gene fragment
  misc_feature 3165..3172: note = restriction endonuclease PmeI recognition site
  misc_feature 3329..3624: note = beta actin antisense strand gene fragment
  misc_feature 3660..3678: note = bacteriophage MS2 pac sequence
  regulatory 3695..3884: note = terminator - yeast CYC1 terminator; regulatory_class = terminator
  misc_feature 5878..7220: note = yeast 2 micron ori
  source 1..7288: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaacgacattactatatatataatataggaagcatttaatagacagcatcgtaatatatgtgtactttgcagttatgacgccagatggcagtagtggaagatattctttattgaaaaatagcttgtcaccttacgtacaatcttgatccggagcttttctttttttgccgattaagaattaattcggtcgaaaaaagaaaaggagagggccaagagggagggcattggtgactattgagcacgtgagtatacgtgattaagcacacaaaggcagcttggagtatgtctgttattaatttcacaggtagttctggtccattggtgaaagtttgcggcttgcagagcacagaggccgcagaatgtgctctagattccgatgctgacttgctgggtattatatgtgtgcccaatagaaagagaacaattgacccggttattgcaaggaaaatttcaagtcttgtaaaagcatataaaaatagttcaggcactccgaaatacttggttggcgtgtttcgtaatcaacctaaggaggatgttttggctctggtcaatgattacggcattgatatcgtccaactgcatggagatgagtcgtggcaagaataccaagagttcctcggtttgccagttattaaaagactcgtatttccaaaagactgcaacatactactcagtgcagcttcacagaaacctcattcgtttattcccttgtttgattcagaagcaggtgggacaggtgaacttttggattggaactcgatttctgactgggttggaaggcaagagagccccgaaagcttacattttatgttagctggtggactgacgccagaaaatgttggtgatgcgcttagattaaatggcgttattggtgttgatgtaagcggaggtgtggagacaaatggtgtaaaagactctaacaaaatagcaaatttcgtcaaaaatgctaagaaataggttattactgagtagtatttatttaagtattgtttgtgcacttgcctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctgaattggagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgcttatttagaagtgtcaacaacgtatctaccaacgatttgacccttttccatcttttcgtaaatttctggcaaggtagacaagccgacaaccttgattggagacttgaccaaacctctggcgaagaattgttaattaagagctcagatcttatcgtcgtcatccttgtaatccatcgatactagtgcggccgccctttagtgagggttgaattcgaattttcaaaaattcttactttttttttggatggacgcaaagaagtttaataatcatattacatggcattaccaccatatacatatccatatacatatccatatctaatcttacttatatgttgtggaaatgtaaagagccccattatcttagcctaaaaaaaccttctctttggaactttcagtaatacgcttaactgctcattgctatattgaagtacggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatccatttaaatacatgaggattacccatgtgcgatcgcgcacgaggtttttctgtctagtgagcagtgtccaacctcaaaagacaacatgtgtgacgacgatgtagcggctcttgtcgtagacaatggatccggtatgtgcaaagccggtttcgcaggagatgacgcaccccgtgccgtcttcccctcgatcgtcggtcgcccaaggcatcaaggagtcatggtcggtatgggacaaaaggactcatacgtaggagatgaagcccaaagcaaaagaggtatcctcaccctgaaataccccatcgaacacggtatcatcaccaactgggatgagtttaaaccctctagctgctttacaaagtactggttccctttccagcgggatgctttatctaaacgcaatgagagaggtattcctcaggccacatcgcttcctagttccgctgggatccatcgttggcggccgaagccgccattccatagtgagttctggcgcgcctcatcccagttggtgatgataccgtgttcgatggggtatttcagggtgaggatacctcttttgctttgggcttcatctcctacgtatgagtccttttgtcccataccgaccatgactccttgatgccttgggcgaccgacgatcgaggggaagacggcacggggtgcgtcatctcctgcgaaaccggctttgcacataccggatccattgtctacgacaagagccgctacatcgtcgtcacacatgttgtcttttgaggttggacactgctcactagacagaaaaacctcgtgccggaccgaatacccggtctgaacgagggcggccgcacatgaggatcacccatgtccgcgggctagctaagatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaagatccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatattaagaaaccattattatcatgacattaacctataaaaataggcgtatcacgaggccctttcgtc

SEQ ID NO: 44 - DNA, 7620 bp, synthetic construct
  misc_feature 1..7620: note = Recombinant plasmid pAPSE10444; beta actin stem loop + coat protein
  regulatory 187..467: note = promoter - yeast TRP1 promoter; regulatory_class = promoter
  gene 468..1142: note = yeast TRP1 gene
  misc_feature 2082..2087: note = restriction endonuclease SacI recognition site
  gene 2091..2480: note = bacteriophage MS2 coat protein gene
  misc_feature 2484..2489: note = restriction endonuclease EcoRI recognition site
  regulatory 2494..3158: note = promoter - yeast GAL1, 10 bidirectional promoter; regulatory_class = promoter
  misc_feature 3176..3194: note = bacteriophage MS2 pac sequence
  misc_feature 3195..3202: note = restriction endonuclease AsiSI recognition sequence
  misc_feature 3204..3497: note = beta actin sense gene fragment
  misc_feature 3497..3504: note = restriction endonuclease PmeI recognition site
  misc_feature 3661..3957: note = beta actin antisense strand gene fragment
  misc_feature 3992..4010: note = bacteriophage MS2 pac sequence
  regulatory 4027..4216: note = terminator - yeast CYC1 terminator; regulatory_class = terminator
  misc_feature 6210..7552: note = yeast 2 micron ori
  source 1..7620: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaacgacattactatatatataatataggaagcatttaatagacagcatcgtaatatatgtgtactttgcagttatgacgccagatggcagtagtggaagatattctttattgaaaaatagcttgtcaccttacgtacaatcttgatccggagcttttctttttttgccgattaagaattaattcggtcgaaaaaagaaaaggagagggccaagagggagggcattggtgactattgagcacgtgagtatacgtgattaagcacacaaaggcagcttggagtatgtctgttattaatttcacaggtagttctggtccattggtgaaagtttgcggcttgcagagcacagaggccgcagaatgtgctctagattccgatgctgacttgctgggtattatatgtgtgcccaatagaaagagaacaattgacccggttattgcaaggaaaatttcaagtcttgtaaaagcatataaaaatagttcaggcactccgaaatacttggttggcgtgtttcgtaatcaacctaaggaggatgttttggctctggtcaatgattacggcattgatatcgtccaactgcatggagatgagtcgtggcaagaataccaagagttcctcggtttgccagttattaaaagactcgtatttccaaaagactgcaacatactactcagtgcagcttcacagaaacctcattcgtttattcccttgtttgattcagaagcaggtgggacaggtgaacttttggattggaactcgatttctgactgggttggaaggcaagagagccccgaaagcttacattttatgttagctggtggactgacgccagaaaatgttggtgatgcgcttagattaaatggcgttattggtgttgatgtaagcggaggtgtggagacaaatggtgtaaaagactctaacaaaatagcaaatttcgtcaaaaatgctaagaaataggttattactgagtagtatttatttaagtattgtttgtgcacttgcctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctgaattggagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgcttatttagaagtgtcaacaacgtatctaccaacgatttgacccttttccatcttttcgtaaatttctggcaaggtagacaagccgacaaccttgattggagacttgaccaaacctctggcgaagaattgttaattaagagctcttattagtaaatgccagagttagcagcgattgcagatgggatcggattaccgtctttcagcaggccctgcattgccttaacaatcagttcgcagtcggagttcgtcgcaaaaatcggaatagtcagttccatgttgaggtaagagcgccaggccgcaactgggagttctacgccgcctacggtctgcgttgcgactttcggtacttcaactttgatggtgtatttgcgattctgcgcgctagactgacgaacggagcacgtgactttgtaggcctgagagcggctgttagagctaatccattccgcaacaccgttggcgaagttggacggagctacggtaacgtcacccgtaccgccgttatcaaccagaacgaattgggtaaagttagacgccatgaattcgaattttcaaaaattcttactttttttttggatggacgcaaagaagtttaataatcatattacatggcattaccaccatatacatatccatatacatatccatatctaatcttacttatatgttgtggaaatgtaaagagccccattatcttagcctaaaaaaaccttctctttggaactttcagtaatacgcttaactgctcattgctatattgaagtacggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatccatttaaatacatgaggattacccatgtgcgatcgcgcacgaggtttttctgtctagtgagcagtgtccaacctcaaaagacaacatgtgtgacgacgatgtagcggctcttgtcgtagacaatggatccggtatgtgcaaagccggtttcgcaggagatgacgcaccccgtgccgtcttcccctcgatcgtcggtcgcccaaggcatcaaggagtcatggtcggtatgggacaaaaggactcatacgtaggagatgaagcccaaagcaaaagaggtatcctcaccctgaaataccccatcgaacacggtatcatcaccaactgggatgagtttaaaccctctagctgctttacaaagtactggttccctttccagcgggatgctttatctaaacgcaatgagagaggtattcctcaggccacatcgcttcctagttccgctgggatccatcgttggcggccgaagccgccattccatagtgagttctggcgcgcctcatcccagttggtgatgataccgtgttcgatggggtatttcagggtgaggatacctcttttgctttgggcttcatctcctacgtatgagtccttttgtcccataccgaccatgactccttgatgccttgggcgaccgacgatcgaggggaagacggcacggggtgcgtcatctcctgcgaaaccggctttgcacataccggatccattgtctacgacaagagccgctacatcgtcgtcacacatgttgtcttttgaggttggacactgctcactagacagaaaaacctcgtgccggaccgaatacccggtctgaacgagggcggccgcacatgaggatcacccatgtccgcgggctagctaagatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaagatccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatattaagaaaccattattatcatgacattaacctataaaaataggcgtatcacgaggccctttcgtc

SEQ ID NO: 45 - DNA, 7288 bp, synthetic construct
  misc_feature 1..7288: note = Recpmbinant plasmid pAPSE10445; beta actin stem loop - coat protein
  regulatory 187..467: note = promoter - yeast TRP1 promoter; regulatory_class = promoter
  gene 468..1142: note = yeast TRP1 gene
  misc_feature 2082..2087: note = restriction endonuclease SacI recognition site
  misc_feature 2152..2157: note = restriction endonuclease EcoRI recognition site
  misc_feature 2162..2826: note = yeast GAL1, 10 bidirectional prmoter
  misc_feature 2844..2862: note = bacteriophage MS2 pac sequence
  misc_feature 2863..2870: note = restriction endonuclease AsiSI recognition sequence
  misc_feature 2872..3165: note = beta actin sense gene fragment
  misc_feature 3165..3172: note = restriction endonuclease PmeI recognition site
  misc_feature 3233..3251: note = bacteriophage MS2 pac sequence
  misc_feature 3329..3624: note = beta actin antisense strand gene fragment
  misc_feature 3660..3678: note = bacteriophage MS2 pac sequence
  regulatory 3695..3884: note = terminator - yeast CYC1 terminator; regulatory_class = terminator
  misc_feature 5878..7220: note = yeast 2 micron ori
  source 1..7288: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaacgacattactatatatataatataggaagcatttaatagacagcatcgtaatatatgtgtactttgcagttatgacgccagatggcagtagtggaagatattctttattgaaaaatagcttgtcaccttacgtacaatcttgatccggagcttttctttttttgccgattaagaattaattcggtcgaaaaaagaaaaggagagggccaagagggagggcattggtgactattgagcacgtgagtatacgtgattaagcacacaaaggcagcttggagtatgtctgttattaatttcacaggtagttctggtccattggtgaaagtttgcggcttgcagagcacagaggccgcagaatgtgctctagattccgatgctgacttgctgggtattatatgtgtgcccaatagaaagagaacaattgacccggttattgcaaggaaaatttcaagtcttgtaaaagcatataaaaatagttcaggcactccgaaatacttggttggcgtgtttcgtaatcaacctaaggaggatgttttggctctggtcaatgattacggcattgatatcgtccaactgcatggagatgagtcgtggcaagaataccaagagttcctcggtttgccagttattaaaagactcgtatttccaaaagactgcaacatactactcagtgcagcttcacagaaacctcattcgtttattcccttgtttgattcagaagcaggtgggacaggtgaacttttggattggaactcgatttctgactgggttggaaggcaagagagccccgaaagcttacattttatgttagctggtggactgacgccagaaaatgttggtgatgcgcttagattaaatggcgttattggtgttgatgtaagcggaggtgtggagacaaatggtgtaaaagactctaacaaaatagcaaatttcgtcaaaaatgctaagaaataggttattactgagtagtatttatttaagtattgtttgtgcacttgcctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctgaattggagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgcttatttagaagtgtcaacaacgtatctaccaacgatttgacccttttccatcttttcgtaaatttctggcaaggtagacaagccgacaaccttgattggagacttgaccaaacctctggcgaagaattgttaattaagagctcagatcttatcgtcgtcatccttgtaatccatcgatactagtgcggccgccctttagtgagggttgaattcgaattttcaaaaattcttactttttttttggatggacgcaaagaagtttaataatcatattacatggcattaccaccatatacatatccatatacatatccatatctaatcttacttatatgttgtggaaatgtaaagagccccattatcttagcctaaaaaaaccttctctttggaactttcagtaatacgcttaactgctcattgctatattgaagtacggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatccatttaaatacatgaggattacccatgtgcgatcgcgcacgaggtttttctgtctagtgagcagtgtccaacctcaaaagacaacatgtgtgacgacgatgtagcggctcttgtcgtagacaatggatccggtatgtgcaaagccggtttcgcaggagatgacgcaccccgtgccgtcttcccctcgatcgtcggtcgcccaaggcatcaaggagtcatggtcggtatgggacaaaaggactcatacgtaggagatgaagcccaaagcaaaagaggtatcctcaccctgaaataccccatcgaacacggtatcatcaccaactgggatgagtttaaaccctctagctgctttacaaagtactggttccctttccagcgggatgctttatctaaacgcaacatgaggatcacccatgtcgccacatcgcttcctagttccgctgggatccatcgttggcggccgaagccgccattccatagtgagttctggcgcgcctcatcccagttggtgatgataccgtgttcgatggggtatttcagggtgaggatacctcttttgctttgggcttcatctcctacgtatgagtccttttgtcccataccgaccatgactccttgatgccttgggcgaccgacgatcgaggggaagacggcacggggtgcgtcatctcctgcgaaaccggctttgcacataccggatccattgtctacgacaagagccgctacatcgtcgtcacacatgttgtcttttgaggttggacactgctcactagacagaaaaacctcgtgccggaccgaatacccggtctgaacgagggcggccgcacatgaggatcacccatgtccgcgggctagctaagatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaagatccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatattaagaaaccattattatcatgacattaacctataaaaataggcgtatcacgaggccctttcgtc

SEQ ID NO: 46 - DNA, 7620 bp, synthetic construct
  misc_feature 1..7620: note = Recombinant plasmid pAPSE10446; beta actin stem loop + coat protein
  regulatory 187..467: note = promoter - yeast TRP1 promoter; regulatory_class = promoter
  gene 468..1142: note = yeast TRP1 gene
  misc_feature 2082..2087: note = restriction endonuclease SacI recognition site
  gene 2091..2480: note = bacteriophage MS2 coat protein gene
  misc_feature 2484..2489: note = restriction endonuclease EcoRI recognition site
  regulatory 2494..3158: note = promoter - yeast GAL1, 10 bidirectional promoter; regulatory_class = promoter
  misc_feature 3176..3194: note = bacteriophage MS2 pac sequence
  misc_feature 3195..3202: note = restriction endonuclease AsiSI recognition sequence
  misc_feature 3204..3497: note = beta actin sense gene fragment
  misc_feature 3497..3504: note = restriction endonuclease PmeI recognition site
  misc_feature 3565..3583: note = bacteriophage MS2 pac sequence
  misc_feature 3662..3956: note = beta actin antisense strand gene fragment
  misc_feature 3992..4010: note = bacteriophage MS2 pac sequence
  regulatory 4027..4216: note = terminator - yeast CYC1 terminator; regulatory_class = terminator
  misc_feature 6210..7552: note = yeast 2 micron ori
  source 1..7620: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaacgacattactatatatataatataggaagcatttaatagacagcatcgtaatatatgtgtactttgcagttatgacgccagatggcagtagtggaagatattctttattgaaaaatagcttgtcaccttacgtacaatcttgatccggagcttttctttttttgccgattaagaattaattcggtcgaaaaaagaaaaggagagggccaagagggagggcattggtgactattgagcacgtgagtatacgtgattaagcacacaaaggcagcttggagtatgtctgttattaatttcacaggtagttctggtccattggtgaaagtttgcggcttgcagagcacagaggccgcagaatgtgctctagattccgatgctgacttgctgggtattatatgtgtgcccaatagaaagagaacaattgacccggttattgcaaggaaaatttcaagtcttgtaaaagcatataaaaatagttcaggcactccgaaatacttggttggcgtgtttcgtaatcaacctaaggaggatgttttggctctggtcaatgattacggcattgatatcgtccaactgcatggagatgagtcgtggcaagaataccaagagttcctcggtttgccagttattaaaagactcgtatttccaaaagactgcaacatactactcagtgcagcttcacagaaacctcattcgtttattcccttgtttgattcagaagcaggtgggacaggtgaacttttggattggaactcgatttctgactgggttggaaggcaagagagccccgaaagcttacattttatgttagctggtggactgacgccagaaaatgttggtgatgcgcttagattaaatggcgttattggtgttgatgtaagcggaggtgtggagacaaatggtgtaaaagactctaacaaaatagcaaatttcgtcaaaaatgctaagaaataggttattactgagtagtatttatttaagtattgtttgtgcacttgcctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctgaattggagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgcttatttagaagtgtcaacaacgtatctaccaacgatttgacccttttccatcttttcgtaaatttctggcaaggtagacaagccgacaaccttgattggagacttgaccaaacctctggcgaagaattgttaattaagagctcttattagtaaatgccagagttagcagcgattgcagatgggatcggattaccgtctttcagcaggccctgcattgccttaacaatcagttcgcagtcggagttcgtcgcaaaaatcggaatagtcagttccatgttgaggtaagagcgccaggccgcaactgggagttctacgccgcctacggtctgcgttgcgactttcggtacttcaactttgatggtgtatttgcgattctgcgcgctagactgacgaacggagcacgtgactttgtaggcctgagagcggctgttagagctaatccattccgcaacaccgttggcgaagttggacggagctacggtaacgtcacccgtaccgccgttatcaaccagaacgaattgggtaaagttagacgccatgaattcgaattttcaaaaattcttactttttttttggatggacgcaaagaagtttaataatcatattacatggcattaccaccatatacatatccatatacatatccatatctaatcttacttatatgttgtggaaatgtaaagagccccattatcttagcctaaaaaaaccttctctttggaactttcagtaatacgcttaactgctcattgctatattgaagtacggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatccatttaaatacatgaggattacccatgtgcgatcgcgcacgaggtttttctgtctagtgagcagtgtccaacctcaaaagacaacatgtgtgacgacgatgtagcggctcttgtcgtagacaatggatccggtatgtgcaaagccggtttcgcaggagatgacgcaccccgtgccgtcttcccctcgatcgtcggtcgcccaaggcatcaaggagtcatggtcggtatgggacaaaaggactcatacgtaggagatgaagcccaaagcaaaagaggtatcctcaccctgaaataccccatcgaacacggtatcatcaccaactgggatgagtttaaaccctctagctgctttacaaagtactggttccctttccagcgggatgctttatctaaacgcaacatgaggatcacccatgtcgccacatcgcttcctagttccgctgggatccatcgttggcggccgaagccgccattccatagtgagttctggcgcgcctcatcccagttggtgatgataccgtgttcgatggggtatttcagggtgaggatacctcttttgctttgggcttcatctcctacgtatgagtccttttgtcccataccgaccatgactccttgatgccttgggcgaccgacgatcgaggggaagacggcacggggtgcgtcatctcctgcgaaaccggctttgcacataccggatccattgtctacgacaagagccgctacatcgtcgtcacacatgttgtcttttgaggttggacactgctcactagacagaaaaacctcgtgccggaccgaatacccggtctgaacgagggcggccgcacatgaggatcacccatgtccgcgggctagctaagatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaagatccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatattaagaaaccattattatcatgacattaacctataaaaataggcgtatcacgaggccctttcgtc

(19) United States
(12) Patent Application Publication
(10) Pub. No.: US 2026/0305618 A1
(43) Pub. Date: Oct. 8, 2026

(54) METHODS AND COMPOSITIONS FOR MICROBIAL DELIVERY OF DOUBLE STRANDED RNA

(71) Applicant: RNAISSANCE AG LLC, Shawnee Mission, KS (US)

(72) Inventors: John L. Killmer, St. Louis, MO (US); Anil Kumar, St. Louis, MO (US)

(21) Appl. No.: 19/717,806

(22) Filed: Jun. 24, 2026

Related U.S. Application Data
(62) Division of application No. 17/615,516, filed on Nov. 30, 2021, now Pat. No. 12,690,596.

(51) Int. Cl. A01N 63/60; A01N 63/20; A01N 63/50; A01P 7/04; C12N 1/20; C12N 15/113; C12N 15/70

(52) U.S. Cl. CPC A01N 63/60; A01N 63/20; A01N 63/50; A01P 7/04; C12N 1/20; C12N 15/113; C12N 15/70; C12N 2310/14; C12N 2310/531; C12N 2795/18022; C12N 2795/18023; C12N 2795/18042; C12N 2795/18122; C12N 2795/18123; C12N 2795/18142

(57) ABSTRACT

Methods and materials for improved delivery of dsRNA are presented. In particular, methods for controlling an insect pest by stably delivering large quantities of dsRNA to the insect pest are provided. Compositions comprising a bacterium containing a large quantity of dsRNA targeting an insect gene in which the dsRNA exhibits increased stability and activity are also provided.

### CROSS-REFERENCE TO RELATED APPLICATIONS

This application is a divisional of U.S. patent application Ser. No. 17/615,516, filed Nov. 30, 2021, which is a U.S. National Stage Application of International Application No. PCT/US2020/035339, filed May 29, 2020, which claims the benefit of U.S. Provisional Patent Application No. 62/854,831, filed May 30, 2019, the entire contents of which are incorporated herein by reference.

### REFERENCE TO SEQUENCE LISTING

The application contains a Sequence Listing which has been submitted electronically in .xml format and is hereby incorporated by reference in its entirety. Said .xml copy, created on Jun. 11, 2026, is named “066803-894485_SeqListing.xml” and is 546,757 bytes in size.

### FIELD OF THE INVENTION

The present invention relates to methods and compositions for delivering double-stranded RNA to an insect that provide enhanced environmental stability of the dsRNA. The methods comprise contacting an insect with a composition comprising a bacterial cell comprising a dsRNA molecule which specifically inhibits expression of a target gene in the insect.

### BACKGROUND OF THE INVENTION

RNA intereference (RNAi) induced through double stranded RNA (dsRNA) has been used to study gene function in insects. Recently, gene knockown by RNAi has shown promise in managing insect pests and is advantageous over chemical pesticides due to its ability to selectively target only pest species and spare beneficial insects.

To activate the RNAi pathway, dsRNA can be fed to insects and absorbed in the cells that line the midgut. Exogenous dsRNA is usually processed into 20-30 nucleotide duplexes by the ribonuclease III enzyme DICER. These nucleotide duplexes are incorporated into the RNA induced silencing complex (RISC) by the catalytic component ARGONAUTE. The two strands of RNA are unwound and one strand is used as a guide strand. The guide strand binds to complementary mRNAs and the RISC complex mediates degradation or suppression of the endogenous transcript. Targeting endogenous transcripts that code for critical genes in the insect pest enables the population to be controlled.

Delivery of intact dsRNA into insect cells, however, remains problematic. Transgenic plants expressing dsRNAs have been used to control plant viral pathogens; however, the use of RNAi in insect pest control has lagged behind following years of unsuccessful attempts. Recently, transgenic plants have been engineered to express insect dsRNAs to target insect pests. Alternatively, dsRNA has been synthesized in vitro and sprayed onto plant parts.

Attempts have been made to utilize yeast as a delivery system for dsRNA; however, although some gene knock-down has been shown using that system, gel-based evidence of dsRNA stability in that system has never been shown, likely because the dsRNA is degraded very rapidly into very short (e.g. 21mer to 30mer) sequences and/or the quantity of dsRNA produced is miniscule.

Despite these advances, microorganisms such as bacteria have not been shown to be useful as a delivery vehicle for dsRNA due to the high concentrations of dsRNA required to initiate the RNAi pathway and due to the environmental instability of dsRNA. In particular, delivery of dsRNA to members of the order Lepidoptera is particularly problematic as they are generally more refractive to RNAi than members of the order Coleoptera.

### SUMMARY OF THE INVENTION

The invention described in the following embodiments provides methods and compositions for delivering large quantities of dsRNA to an insect pest in order to control the insect pest. In some aspects, the compositions comprise a bacterial cell (killed or living) comprising a large quantity of dsRNA that specifically inhibits expression of a target gene in an insect pest, wherein the dsRNA exhibits increased stability over time compared to the same dsRNA in a different composition or compared to the same dsRNA when “naked”. In some aspects, dsRNA in the composition remains environmentally stable over a period of at least 7 days in, e.g. insect saliva, puddle water, pond water and/or on the surface of a leaf. In some aspects, the methods comprise contacting an insect pest with a composition comprising a bacterial cell comprising a large quantity of dsRNA that specifically inhibits expression of a target gene in an insect pest, thereby controlling the insect pest.

The bacterial cell can be any bacteria capable of transformation. In some aspects, the bacterial cell lacks double-strand specific RNase III and/or contains an inducible T7 RNA polymerase gene. In some aspects, the bacterial cell is a gram negative bacterial cell, such as an E. coli strain, representative examples of which include K12 strains and derivatives thereof (e.g. MG1655, HT115(DE3)) and B strains (e.g. BL21(DE3), REL606). In some aspects, the bacterial cell is a gram positive bacterial cell. In a preferred aspect, the gram-positive bacterial cell is from the genus Corynebacterium, more preferably is Corynebacterium glutamicum.

Delivery of dsRNA should induce a lethal phenotype in the insect pest. Suitable genes to be targeted by dsRNA in the insect pest include midgut or non-midgut genes. Representative non-limiting examples of suitable insect genes that can be targeted by dsRNA include tubulin, vATPase, acetyl choline esterase, chitin synthase gene A, beta-actin, and genes coding for inhibitors of apoptosis (e.g. IAP).

The term “insect” should be understood to refer not only to insects but to their immature forms and larvae.

In some embodiments, the insect pest to be controlled is selected from the orders Coleoptera, Diptera, Hymenoptera, Lepidoptera, Mallophaga, Homoptera, Orthoptera, Thysanoptera, Dermaptera, Isoptera, Anoplura, Siphonaptera, and Trichoptera. In preferred embodiments, the insect pest to be controlled is a member of the order Coleoptera or Lepidoptera.

Larvae and adults of the order Lepidoptera include, but are not limited to, armyworms, cutworms, loopers and heliothines Larvae of the order Lepidoptera include, but are not limited to, armyworms, cutworms, loopers, and heliothines in the family Noctuidae Spodoptera frugiperda JE Smith (fall armyworm); S. exigua. Hubner (beet armyworm)/S. litura Fabricius (tobacco cutworm, cluster caterpillar) Mamestra configurata Walker (bertha armyworm); M. brassicae Linnaeus (cabbage moth); Agrotis ipsilon Hufnagel (black cutworm); A. orthogonia Morrison (western cutworm); A. subterranea Fabricius (granulate cutworm); Alabama argillacea Hubner (cotton leaf worm); Trichoplusia ni Hubner (cabbage looper); Pseudoplusia includens Walker (soybean looper); Anticarsia gemmatalis Hubner (velvetbean caterpillar); Hypena scabra Fabricius (green cloverworm); Heliothis virescens Fabricius (tobacco budworm); Pseudaletia unipuncta Haworth (armyworm); Athetis mindara Barnes and Mcdunnough (rough skinned cutworm); Euxoa messona Harris (darksided cutworm); Earias insulana Boisduval (spiny bollworm); E. vittella Fabricius (spotted bollworm) Helicoverpa armigera Hubner (American bollworm); H. zea Boddie (corn earworm or cotton bollworm); Melanchra picta Harris (zebra caterpillar); Egira (Xylomyges) curialis Grote (citrus cutworm); borers, casebearers, webworms, coneworms, and skeletonizers from the family Pyralidae Ostrinia nubilalis Hubner (European corn borer); Amyelois transitella Walker (naval orangeworm); Anagasta kuehniella Zeller (Mediterranean flour moth); Cadra cautella Walker (almond moth); Chilo suppressalis Walker (rice stem borer); C. partellus, (sorghum borer); Corcyra cephalonica Stainton (rice moth); Crambus caliginosellus Clemens (corn root webworm); C. teterrellus Zincken (bluegrass webworm); Cnaphalocrocis medinalis Guenee (rice leaf roller); Desmia funeralis Hubner (grape leaffolder); Diaphania hyalinata Linnaeus (melon worm); D. nitidalis Stoll (pickleworm); Diatraea grandiosella Dyar (southwestern corn borer); D. saccharalis Fabricius (surgarcane borer) Eoreuma loftini Dyar (Mexican rice borer); Ephestia elutella Hubner (tobacco (cacao) moth) Galleria mellonella Linnaeus (greater wax moth); Herpetogramma licarsisalis Walker (sod webworm); Homoeosoma electellum Hulst (sunflower moth); Elasmopalpus lignosellus Zeller (lesser cornstalk borer); Achroia gnsella Fabricius (lesser wax moth); Loxostege sticticalis Linnaeus (beet webworm); Orthaga thyrisalis Walker (tea tree web moth); Maruca testulalis Geyer (bean pod borer); Plodia interpunctella Hubner (Indian meal moth); Scirpophaga incertulas Walker (yellow stem borer); Udea rubigalis Guenee (celery leaftier); and leafrollers, budworms, seed worms, and fruit worms in the family Tortricidae Acleris gloverana Walsingham (Western blackheaded budworm); A. variana Fernald (Eastern blackheaded budworm); Archips argyrospila Walker (fruit tree leaf roller); A. rosana Linnaeus (European leaf roller); and other Archips species, Adoxophyes orana Fischer von Rosslerstamm (summer fruit tortrix moth); Cochylis hospes Walsingham (banded sunflower moth); Cydia latiferreana Walsingham (filbertworm); C. pomonella Linnaeus (coding moth); Platynota flavedana Clemens (variegated leafroller); P. stultana Walsingham (omnivorous leafroller); Lobesia botrana Denis & Schiffermiiller (European grape vine moth); Spilonota ocellana Denis & Schiffermiiller (eyespotted bud moth); Endopiza viteana Clemens (grape berry moth); Eupoecilia ambiguella Hubner (vine moth); Bonagota salubncola Meyrick (Brazilian apple leafroller); Grapholita molesta Busck (oriental fruit moth); Suleima helianthana Riley (sunflower bud moth); Argyrotaenia spp.; Choristoneura spp. Alsophila pometaria Harris (fall cankerworm); Anarsia lineatella Zeller (peach twig borer); Anisota senatoria J. E. Smith (orange striped oakworm); Antheraea pernyi Guerin-Meneville (Chinese Oak Tussah Moth); Bombyx mori Linnaeus (Silkworm); Bucculatfix thurbefiella Busck (cotton leaf perforator); Colias eurytheme Boisduval (alfalfa caterpillar); Datana integerrima Grote & Robinson (walnut caterpillar); Dendrolimus sibiricus Tschetwerikov (Siberian silk moth), Ennomos subsignaria Hubner (elm spanworm); Erannis tiliaria Harris (linden looper); Euproctis chrysorrhoea Linnaeus (browntail moth); Harrisina americana Guerin-Meneville (grapeleaf skeletonizer); Hemileuca oliviae Cockrell (range caterpillar); Hyphantria cunea Drury (fall webworm); Keiferia lycopersicella Walsingham (tomato pinworm); Lambdina fiscellaria Hulst (Eastern hemlock looper); L. fiscellaria l.ugubrosa Hulst (Western hemlock looper); Leucoma salicis Linnaeus (satin moth); Lymantria dispar Linnaeus (gypsy moth); Manduca quinquemaculata Haworth (five spotted hawk moth, tomato hornworm); M. sexta Haworth (tomato hornworm, tobacco hornworm); Operophtera brumata Linnaeus (winter moth); Paleacrita vernata Peck (spring cankerworm); Papilio cresphontes Cramer (giant swallowtail, orange dog); Phryganidia californica Packard (California oakworm); Phyllocnistis citrella Stainton (citrus leafminer); Phyllonorycter blancardella Fabricius (spotted tentiform leafminer); Pieris brassicae Linnaeus (large white butterfly); P. rapae Linnaeus (small white butterfly); P. napi Linnaeus (green veined white butterfly); Platyptilia carduidactyla Riley (artichoke plume moth); Plutella xylostella Linnaeus (diamondback moth); Pectinophora gossypiella Saunders (pink bollworm); Pontia protodice Boisduval & Leconte (Southern cabbageworm); Sabulodes aegrotata Guenee (omnivorous looper); Schizura concinna J. E. Smith (red humped caterpillar); Sitotroga cerealella Olivier (Angoumois grain moth); Thaumetopoea pityocampa Schiffermuller (pine processionary caterpillar); Tineola bisselliella Hummel (webbing clothesmoth); Tuta absoluta Meyrick (tomato leafminer); Yponomeuta padella Linnaeus (ermine moth); Heliothis subflexa Guenee; Malacosoma spp. and Orgyia spp. Tenebhonidae.

Larvae and adults of the order Coleoptera include weevils from the families Anthribidae, Bruchidae, and Curculionidae (including, but not limited to: Anthonomus grandis Boheman (boll weevil); Lissorhoptrus oryzophilus Kuschel (rice water weevil); Sitophilus granarius Linnaeus (granary weevil); S. oryzae Linnaeus (rice weevil); Hypera punctata Fabricius (clover leaf weevil); Cylindrocopturus adspersus LeConte (sunflower stem weevil); Smicronyx fulvus LeConte (red sunflower seed weevil); S. sordidus LeConte (gray sunflower seed weevil); Sphenophorus maidis Chittenden (maize billbug); flea beetles, cucumber beetles, rootworms, leaf beetles, potato beetles, and leafminers in the family Chrysomelidae (including, but not limited to: Leptinotarsa decemlineata Say (Colorado potato beetle); Diabrotica virgifera LeConte (western corn rootworm); D. barberi Smith & Lawrence (northern corn rootwormj; D. undecimpunctata howardi Barber (southern corn rootworm); Chaetocnema pulicaria Melsheimer (corn flea beetle); Phyllotreta cruciferae Goeze (corn flea beetle); Colaspis brunnea Fabricius (grape colaspis); Oulema melanopus Linnaeus (cereal leaf beetle); Zygogramma exclamationis Fabricius (sunflower beetle)); beetles from the family Coccinellidae (including, but not limited to: Epilachna vailvestis Mulsant (Mexican bean beetle)); chafers and other beetles from the family Scarabaeidae (including, but not limited to: Popilliajaponica Newman (Japanese beetle); Cyclocephala borealis Arrow (northern masked chafer, white grub); C. immaculata Olivier (southern masked chafer, white grub); Rhizotrogus majalis Razoumowsky (European chafer); Phyllophaga crinita Burmeister (white grub); Ligyrus gibbosus De Geer (carrot beetle)), carpet beetles from the family Dermestidae; wireworms from the family Elatehdae, Eleodes spp., Melanotus spp.; Conoderus spp.; Limonius spp.; Agriotes spp.; Ctenicera spp.; Aeolus spp.; bark beetles from the family Scolytidae and beetles from the family Tenebhonidae.

In some other embodiments, the insect pest to be controlled is a member of the order Hymenoptera such as an ant, sawfly, wasp or bee. In related aspects, the insect pest to be controlled is an ant, preferably selected from Solenopsis invicta (fire ants), Camponotus pennsylvanicus and Camponotus floridanus (carpenter ants), Linepithema humile (Argentine ants), Tapinoma sessile (odorous ants), Tetramorium caespitum (pavement ants), and Monomorium pharaonis (pharaoh ants).

In other embodiments, the insect pest to be controlled is a member of the order Diptera such as a mosquito or fly, e.g. A. gambiae (malaria mosquito) or Ae. aegypti (yellow fever mosquito).

In other embodiments, the insect pest to be controlled is a member of the order Acari (e.g. ticks), Blattodea (cockroach), Dermaptera (earwigs), Heteroptera (e.g. bed bug), Isoptera (termite), Siphonaptera (flea), Sternorrhyncha (aphids), or Zygentoma (silverfish)

RNAi strategies for reducing or inhibiting expression of a target gene are known in the art and employ genetic constructs that encode double-stranded RNA (dsRNA). Typically, such constructs comprise sense and anti-sense sequences which are placed in regions flanking an intron sequence in proper splicing orientation with donor and acceptor splicing sites. Alternatively, spacer sequences of various lengths can be employed to separate self-complementary regions of sequence in the construct. During processing of the gene construct transcript, intron sequences can be spliced-out, allowing sense and anti-sense sequences, as well as splice junction sequences, to bind forming double-stranded RNA.

The RNAi polynucleotide can hybridize with the full length mRNA encoded by the target gene or hybridize to a fragment of the target RNA or DNA (the target sequence). The target sequence is typically between 1 and 500 nucleotides in length. In some embodiments, the target sequence and/or the dsRNA sequence is between about 50 and 400 nucleotides in length, preferably between 100 and 300 nucleotides in length. Typically, the sequence of the dsRNA used for RNAi is 100% identical to the target sequence of the target gene, but can be at least 70%, 80%, 90%, 95%, 98% or 99% or more identical to the target sequence.

In some embodiments, compositions for delivering large quantities of dsRNA to an insect pest in order to control the insect pest comprise a bacterial cell containing (i) a gene encoding a self-complementary stretch of sequence separated by non-complementary sequence such that upon hybridization of the complementary sequences a stem-loop structure is formed, wherein the stem portion of the molecule functions as an RNAi precursor when introduced into the target insect pest and (ii) a bacteriophage coat protein gene encoding a capsid protein. Expression of the dsRNA gene and the coat protein gene results in increased accumulation of un-degraded dsRNA and capsid protein, wherein the dsRNA exhibits surprisingly increased stability over time compared to the same dsRNA in a different composition or compared to the same dsRNA when “naked”. In some aspects, stability of the dsRNA is maintained in an environment selected from insect saliva, puddle water, rain water and a leaf surface.

In some embodiments, a composition comprising a bacterial cell comprising dsRNA targeting an insect gene is provided, wherein the dsRNA is stable (i.e. substantially undegraded) for a period of at least 7 days, at least 8 days, at least 9 days, at least 10 days, at least 11 days, at least 12 days, at least 13 days, at least 2 weeks, at least 3 weeks, at least 4 weeks or more. In some aspects, a composition comprising a bacterial cell comprising dsRNA targeting an insect gene is provided wherein the dsRNA exhibits increased stability relative to the same dsRNA when “naked”. In other aspects, a bacterial cell comprising dsRNA targeting an insect gene is provided, wherein at least 50%, at least 60%, at least 70%, at least 80%, at least 90%, at least 95% or more of the dsRNA remains undegraded after a period of 7, 8, 9, 10, 11, 12, 13, 14, 15, 16, 17, 18, 19, 20, 21, 22, 23, 24, 25, 26, 27, 28, 29, 30 or more days.

In related embodiments, a composition comprising a bacterial cell comprising dsRNA targeting an insect gene is provided wherein the dsRNA maintains at least 50%, at least 60%, at least 70%, at least 80%, at least 90%, at least 95% or more of its original RNAi activity for a period of at least 7 days, at least 8 days, at least 9 days, at least 10 days, at least 11 days, at least 12 days, at least 13 days, at least 2 weeks, at least 3 weeks, at least 4 weeks or more.

In one embodiment the bacteriophage capsid protein is encoded by the coat protein gene of a species of leviviridae. In a preferred embodiment the coat protein gene encodes the capsid protein of bacteriophage MS2. In another preferred embodiment the coat protein gene encodes the capsid protein of bacteriophage Qbeta.

In an embodiment the capsid protein comprises the N-terminus of the MS2 capsid protein. In another embodiment the capsid protein comprises the N-terminal 41, 35, 25, 21 or 12 amino acids of the MS2 capsid protein. In an embodiment the capsid protein comprises the N-terminus of the Qbeta capsid protein. In another embodiment the capsid protein comprises the N-terminal 41, 35, 25, 21 or 12 amino acids of the Qbeta capsid protein.

In an embodiment the gene encoding the dsRNA may be associated with and expressed from an inducible transcriptional promoter. The coat protein gene may be associated with and expressed from a constitutive or inducible transcriptional promoter. The inducible transcriptional promoter associated with expression of the dsRNA may be the same inducible transcriptional promoter or a different transcriptional promoter from a transcriptional promoter associated with expression of the coat protein gene. In one embodiment the inducible transcriptional promoter associated with expression of the coat protein gene is induced before induction of the inducible transcriptional promoter associated with expression of the dsRNA to allow accumulation of capsid protein prior to production of dsRNA. In another embodiment the transcriptional promoter associated with expression of the coat protein gene is a constitutive transcriptional promoter.

In an embodiment the gene encoding the dsRNA and the coat protein gene encoding the capsid protein are present on a plasmid or extrachromosomal element. The gene encoding the dsRNA and the coat protein gene may be present on the same plasmid or extrachromosomal element or may be present on separate plasmids or extrachromosomal elements. In another embodiment one or both of the genes encoding the dsRNA and the coat protein may be present on the microbial host cell chromosome or chromosomes.

### DESCRIPTION OF THE DRAWINGS

FIG. 1 (FIG. 1) depicts an RNA stem-loop structure with three pac-site hairpin sequences, one located 5′ of the stem-loop structure, one within the loop of the stem-loop structure, and the other 3′ of the stem-loop structure.

FIG. 2 (FIG. 2) depicts a single strand (sense) sequence flanked on each side by a pac-site hairpin sequence.

FIG. 3 (FIG. 3) depicts a single strand (antisense) sequence flanked on each side by a pac-site hairpin sequence.

FIG. 4 (FIG. 4) depicts an RNA stem-loop structure with two pac-site hairpin sequences. one located 5′ of the stern-loop structure and the other 3′ of the stern-loop structure.

FIG. 5 (FIG. 5) depicts an RNA stem-loop structure with a single pac-site hairpin sequence located 3′ of the stem-loop structure.

FIG. 6 (FIG. 6) depicts an RNA stem loop structure lacking any pac site hairpin sequences.

FIG. 7 (FIG. 7) depicts gels demonstrating stability of dsRNA contained inside C. glutamicum (C.g) cells vs naked dsRNA upon exposure to water from different environments over the specified time periods. A: depicts gels demonstrating stability of naked dsRNA in water from different environments over the specified time periods. B: depicts gels demonstrating stability of dsRNA contained inside C. glutamicum (C.g) cells in water from different environments over the specified time periods. The amount of dsRNA seen in different lanes of the gels was quantitated using the AzureSpot 2.0 software. The amount of dsRNA quantitated at zero hour (0 H) in distilled water (D) was used as control for each gel to calculate the percentage of dsRNA remaining at specified time periods in different environments for both naked dsRNA and the dsRNA contained inside C. glutamicum in that gel. D=Distilled water; T=Tap water; Pu=Puddle water; P=Pond water.

### DETAILED DESCRIPTION OF THE INVENTION

The present invention comprises compositions and methods for delivering large quantities of dsRNA to an insect pest in order to activate the RNAi pathway in the insect. In some aspects, a composition is provided comprising a bacterium that co-expresses a bacteriophage capsid protein, or a portion thereof, in conjunction with the desired dsRNA for a period of time sufficient to allow accumulation of the dsRNA in the bacterium and optionally delivering the composition to an insect pest.

Table 1 outlines a number of permutations of RNA structure and coat protein to optimize the yield of dsRNA produced by the bacterial host. The leftmost column of Table 1 refer to individual figures representing cartoon depiction of the predicted RNA structure produced from each of the listed plasmid constructs. In each figure “S” represents the sense strand, “AS” represents antisense strand, and the small hairpin structures represent pac site sequences). The table also lists the coat protein (if any) and the yields of dsRNA (or ssRNA, as indicated) associated with each of the listed plasmid constructs.

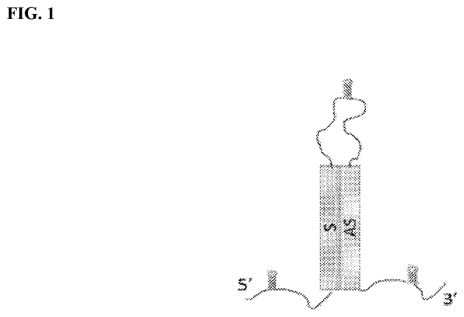

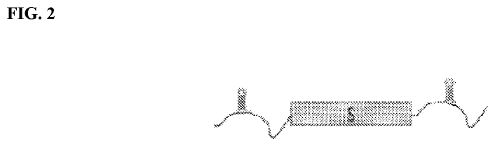

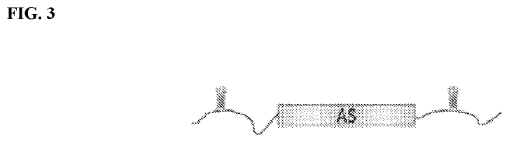

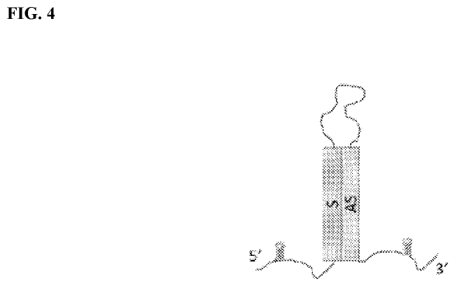

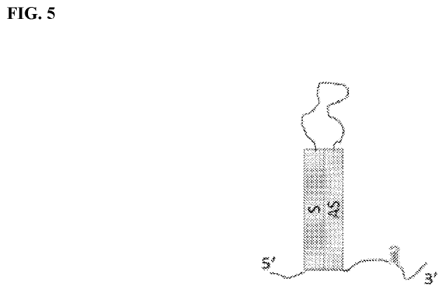

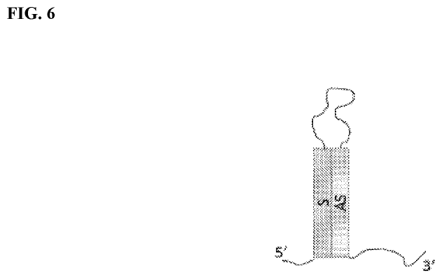

TABLE 1
Production of RNA by E. coli HT115(DE3) as a function of variation in RNA
structure and the presence or absence of coat protein and coat protein variants
(n.a. = not applicable; n.d. = not determined).

| | | Loop | Stem | | | RNA | RNA |
|---|---|---|---|---|---|---|---|
| RNA Structure | | size | size | Stem | Coat | encapsid | excapsid |
| as depicted in | Plasmid | (bases) | (bp) | sequence | protein | (mg/L) | (mg/L) |
| FIG. 1 | pAPSE10180 | 139 | 180 | ErkA | MS2 | <2 | 75-90 |
| FIG. 1 | pAPSE10181 | 139 | 180 | ErkA | none | n.a | <2. |
| FIG. 2 | pAPSE10189 | n.a. | n.a. | beta actin | MS2 | 20 | <2 |
| FIG. 3 | pAPSE10190 | n.a. | n.a. | beta actin | MS2 | 20 | <2 |
| FIG. 2 | pAPSE10274 | n.a. | n.a. | beta actin | none | n.a. | <2 |
| FIG. 3 | pAPSE10275 | n.a. | n.a. | beta actin | none | n.a. | <2 |
| FIG. 1 | pAPSE10269 | 166 | 294 | beta actin | MS2 | 2-10 | 200 |
| FIG. 1 | pAPSE10306 | 166 | 294 | beta actin | none | n.a. | 3 |
| FIG. 4 | pAPSE10216 | 166 | 294 | beta actin | MS2 | 5-20 | 50-250 |
| FIG. 4 | pAPSE10305 | 166 | 294 | beta actin | none | n.a. | 4 |
| FIG. 5 | pAPSE10219 | 166 | 294 | beta actin | MS2 | 5-20 | 30-60 |
| FIG. 5 | pAPSE10304 | 166 | 294 | beta actin | none | n.a. | 3 |
| FIG. 6 | pAPSE10279 | 166 | 294 | beta actin | MS2 | 4 | 65 |
| FIG. 6 | pAPSE10303 | 166 | 294 | beta actin | none | n.a. | 4 |
| FIG. 4 | pAPSE10270 | 116 | 294 | beta actin | MS2 | 2-10 | 200 |
| FIG. 4 | pAPSE10271 | 136 | 294 | beta actin | MS2 | 2-10 | 200 |
| FIG. 4 | pAPSE10272 | 156 | 294 | beta actin | MS2 | 2-10 | 200 |
| FIG. 4 | pAPSE10292 | 131 | 294 | beta actin | MS2 | 2-10 | 150 |
| FIG. 4 | pAPSE10291 | 142 | 294 | beta actin | MS2 | 2-10 | 160 |
| FIG. 4 | pAPSE10276 | 166 | 50 | beta actin | MS2 | 5-10 | 80-120 |
| FIG. 4 | pAPSE10277 | 166 | 75 | beta actin | MS2 | 20-30 | 200-250 |
| FIG. 4 | pAPSE10366 | 166 | 294 | beta actin | none | n.a. | <2 |
| | | | | | (eGFP) | | |
| FIG. 4 | pAPSE10181 | 139 | 180 | ErkA | MS2 | n. d. | 200 |
| | and | | | | in trans | | |
| | pAPSE10149 | | | | | | |
| FIG. 1 | pAPSE10359 | 166 | 294 | beta actin | Qbeta | n.d. | n.d. |
| FIG. 4 | pAPSE10357 | 166 | 294 | beta actin | none | n.d. | n.a. |
| | | | | | (U1A) | | |
| FIG. 1 | pAPSE10372 | 139 | 180 | ErkA | none | n.a. | 75 |
| | | | | | (MS2 | | |
| | | | | | N-term | | |
| | | | | | fragment) | | |

### A. Definitions

As used herein, the term “capsid protein” or “capsid” refers to the coat protein of bacteriophage MS2 or Qβ, capable of binding the bacteriophage RNA pac site with high affinity and assembling into a complex hollow tertiary structure in which the bacteriophage RNA is entirely encapsidated within the hollow tertiary structure. In a VLP, the capsid protein forms a hollow tertiary structure in which the heterologous RNA is entirely encapsidated. The term “capsid” also refers to the hollow tertiary structure formed by assembly of individual capsid proteins.

As used herein “ssRNA” and “dsRNA” refer to “single-stranded RNA and double stranded RNA, respectively. An ssRNA is comprised of an RNA sequence of any length that lacks sufficient internal homology to form any significant secondary structures such as hairpins or other structures dependent on hybridization of internal complementary sequences with one another via Watson-Crick base pairing of nucleotide bases between the complementary sequences. In contrast, a dsRNA comprises RNA sequences with sufficient internal homology to form significant secondary structures such as hairpins due to hybridization of internal complementary sequences with one another via Watson-Crick base pairing of nucleotide bases within the complementary sequences. Significant secondary structures generally involve stretches of homology greater than approximately nine bases, but the exact length depends to some extent on context and on whether such secondary structures impart any biological function to the molecule.

As used herein “plasmid” or “extrachromosomal element” refers to any extrachromosomal episome capable of replication or stable maintenance within the host cell. Specifically embraced by this definition are plasmids such as pBR322, pCG1, and pACYC184 which represent the backbones of the described plasmids. Those of ordinary skill in the art will recognize that other plasmids or stably maintained viral episomes can provide the same required functions of maintenance, expression and selection and that alternatives to the basic plasmids described herein may be generated from such other plasmids or stably maintained viral episomes without undue experimentation. A key feature of the present invention is the ability to express the genes encoding a dsRNA and a capsid protein, not specific modes of replication, expression or the selective markers found on episomes containing the genes encoding the dsRNA and capsid protein.

“Substantially similar sequence” refers to sequence variants of the claimed capsid proteins that retain the ability to facilitate accumulation of dsRNA in a microbial host cell as described herein. Such substantially similar sequences include sequences with at least 26% identity and 47% similarity as shown by the differences between MS2 and Qbeta capsid protein sequences (as determined by blastp). Consequentially, substantially similar sequences encompass conserved and homologous substitutions allowing sequence variants with as little as 95%, 90%, 80%, 70%, 60%, 50%, 40%, 30% or 25% identity to, and 95%, 90%, 80%, 70%, 60%, 50% or 40% similarity to, MS2 or Qbeta capsid protein sequences to facilitate accumulation of dsRNA in a microbial host.

### B. Common Materials and Methods

Routine microbial and molecular cloning methods and tools, including those for generating and purifying DNA, RNA, and proteins, and for transforming host organisms and expressing recombinant proteins and nucleic acids as described herein, are fully within the capabilities of a person of ordinary skill in the art and are well described in the literature. See, e.g., Sambrook, et al., Molecular Cloning: A Laboratory Manual, 2nd ed., Cold Spring Harbor Laboratory Press, Cold Spring Harbor, N.Y. (1989); Davis, et al., Basic Methods in Molecular Biology, Elsevier Science Publishing Co., Inc., N.Y. (1986); and Ausubel, et al, Current Protocols in Molecular Biology, Greene Publ. Assoc., Wiley-Interscience, NY (1995). The disclosures in each of which are herein incorporated by reference.

Each of the recombinant DNA constructs described in further detail below are based on a common plasmid vector series derived from plasmid pBR322. The first of this plasmid vector series contains a custom synthetic DNA fragment (produced by PCR GenScript, Piscataway, NJ) comprising a T7 promoter sequence capable of driving transcription of a single copy of the bacteriophage MS2 capsid gene followed by a T7 terminator. This synthetic sequence was inserted as a BamHl-Sphl restriction fragment into the corresponding sites of pBR322 to form plasmid pAPSE10118. A second synthetic sequence comprising a T7 promoter sequence followed by an MS2 pac site sequence, a multi-cloning site containing, in order (5′ to 3′) AsiSI-Pmel-Ascl-RsrIl-Notl-Pacl restriction sites, a second high affinity variant MS2 pac type sequence (C-pac), a T7 terminator and an SphI restriction site was synthesized (PCR Genscript, Piscataway, NJ) and inserted into the EcoRV site of pAPSE10118 to form pAPSE10136. The two are oriented such that the T7 promoters direct transcription of the same strand of pAPSE10136 (clockwise on the standard pBR322 map) but are separated from one another by a single T7 terminator.

A 180 nucleotide fragment of the ErkA gene of Drosophila melanogaster (corresponding to the sequence of GenBank Accession NM 001300706 between nucleotides 156-335) was amplified by PCR incorporating AsiSI and Pmel restriction sites on the 5′ and 3′ sides, respectively. Insertion of this ErkA gene fragment into the corresponding sites of pAPSE10136 produced pAPSE10169. A second, complimentary copy of the ErkA gene fragment sequence was generated by PCR amplification incorporating a Pmel restriction site on the 5′ end, followed by a synthetic loop sequence containing an additional MS2 pac sequence, followed by a Nod restriction site, followed by the complementary (anti-sense) ErkA gene fragment sequence and a Pad restriction site on the 3′ end of the PCR fragment. The synthetic loop sequence comprises random sequence incapable of hybridizing with the ErkA gene fragment sequences. This complementary (anti-sense) copy of the ErkA gene fragment is inserted into the Pmel and Pad restriction sites of pAPSE10136 to form pAPSE10180 (SEQ ID NO: 1). A second series of plasmid vectors, lacking the MS2 capsid protein is derived from pAPSE10180 by deleting the MS2 capsid expression sequences by SphI restriction digestion and re-ligation to produce pAPSE 10181 (SEQ ID NO: 2).

Plasmids pAPSE10180 and pAPSE10181 represent the basic platform for expression of the RNA constructs discussed herein. Transcription of the ErkA cassette in these plasmids is predicted to produce an RNA transcript capable of forming a large stem-loop structure comprising a 180 base pair stem and a 139 base loop with 3 individual MS2 pac sequences located 5′ and 3′ of the stem and within the loop itself. One of ordinary skill in the art will understand that substitution of the ErkA gene fragment sequences by other sequences can be easily accomplished by standard cloning and sub-cloning methods.

Transformation of plasmids pAPSE10180 or pAPSE10181, or any of their derivatives, into host cells capable of inducible expression of T7 polymerase produces cell lines capable of expressing RNA transcripts. All such strains inducibly producing RNA transcripts are referred to generally herein as “expression strains”. Unless otherwise indicated, each of the plasmids described herein was electroporated into E. coli strain HT115(DE3) with genotype F. mcrA,mcrB, IN (rrnD-rrnE)1, rnc14::Tn10 (Lambda DE3 lysogen: lacUV5 promoter-T7 polymerase)) and the resulting recombinant transformants were selected on LB agar plates containing 12 μg/ml tetracycline and/or 100 μg/ml ampicillin. Single colonies were isolated, the presence of intact plasmid confirmed by restriction enzyme analysis and the confirmed transformed cells archived for future use.

Standard expression studies comprised inoculating transformed cells into 100 ml of Super Broth containing 0.1% glucose, 0.4% lactose, 100 μg/ml ampicillin and/or 12.5 μg/ml tetracycline and incubating the cultures with vigorous shaking at 37° C. Expression of the T7 polymerase was achieved by auto-induction by depletion of the available glucose and the presence of the lactose inducer. This ensures that all cultures are induced at the same growth stage. Cells were harvested twelve to eighteen hours post-induction (late stationary phase) by centrifugation at 3,000 g at 4° C. for 30 minutes and stored on ice until lysis.

RNA was isolated from harvested cells by resuspending a 5 ml equivalent of cell culture of harvested cells in sonication buffer comprising Tris-HCl pH 7, 10 mM NaCl and sonicating the suspended cells on ice for 3 minutes. Cell debris was removed by centrifugation at 16,000 g the supernatant (cleared lysate) was immediately processed to recover RNA and VLPs as described. RNA was recovered from half of the cleared lysate using the commercial Purelink RNA Mini Kit method (Ambion® Cat. No. 12183018A, Thermo Fisher Scientific® Inc., Waltham, MA) according to the manufacturer's instructions.

VLPs were purified from the remaining half of the cleared lysate which were diluted to a total volume of 1 ml and treated with 100 units of Benzonase® Nuclease (Sigma Aldrich®, St. Louis, MO) at 37° C. for two hours. Subsequently, 0.15 mg of Proteinase K was added and the enzymatically treated cleared lysate incubated at 37° C. for an additional three hours. The VLPS were recovered from the enzymatically treated cleared lysate by fractional precipitation. A saturated ammonium sulfate solution was prepared by adding ammonium sulfate to water until it reached saturation (approximately 4.1 M). Fifty microliters of the saturated ammonium sulfate solution was added to the enzymatically treated cleared lysate and the mixture placed on ice and incubated for two hours. Unwanted precipitate was removed from the mixture by centrifugation at 16,000 g and the aqueous solution transferred to a clean Eppendorf tube. The aqueous solution was then subjected to a second precipitation by the addition of 0.171 g of dry ammonium sulfate directly to the aqueous solution. The aqueous solution was vortexed and incubated on ice for two hours. The precipitate was spun down at 16,000 g the aqueous phase discarded and the solid precipitate representing purified VLPs resuspended in 100 microliters of sonication buffer.

RNA was recovered from the resuspended purified VLPs by adding 3 volumes of TRIzol™ LS Reagent (Ambion® Cat. No. 10296028, Thermo Fisher Scientific® Inc.), vigorously vortexing the mixture, adding 1 ml of chloroform, further vortexing the mixture before pulse centrifugation to separate the aqueous and organic phases of the mixture. The aqueous phase was placed in a clean Eppendorf tube and the RNA purified with a commercial RNA Clean & Concentrator™ kit (Cat. No. R1018, Zymo Research®, Irvine, CA) according to the manufacturer's instructions.

RNA from bacterial and VLP samples were dissolved in 50 μl of nuclease-free water. To determine the concentration of dsRNA in a sample, the samples were treated with RNAse A (Invitrogen® Cat. No. AM2274, Thermo Fisher Scientific® Inc.) to degrade single stranded RNA under the manufacturers recommended conditions, the concentration of dsRNA was determined spectrophotmetrically by measuring OD26o and 1 jig loaded onto Novex® 6% TBE-urea gels (Invitrogen®, Thermo Fisher Scientific® Inc.). One lane of each gel was loaded with dsDNA size markers of known concentration and the samples were electrophoresed, the gel was stained with ethidium bromide and each band quantitated by densitometry using the dsDNA markers as a standard curve.

RNA yields from constructs producing ssRNA were determined by annealing the sense or anti-sense strand recovered from the induced cells or VLPs with an excess of the cognate strand. The annealed mixture was then treated with RNAse A and the amount of dsRNA incorporating the ssRNA of interest measured as described above.

Little or no differences in final cell densities were observed between any of the cultures from which the samples were harvested and in all cases the cultures appear to have reached stationary phase prior to harvest. To allow direct sample to sample comparison of RNA yields, all dsRNA and ssRNA concentrations are reported as the amount of such RNA present in a 1 L equivalent of culture.

Northern blot analysis was used to verify the identity of bands containing the dsRNA transcripts using a DNA oligonucleotide probe against the random sequence comprising the loop of each dsRNA construct (5′-GGCCGGCGTCT-ATTAGTAGATGCC-3′, SEQ ID NO 3). RNA from the 6% polyacrylamide denaturing Urea-TBE gel was transferred to a positively-charged BrightStar Plus nylon membrane (Ambion® Cat. No. 10102, Thermo Fisher Scientific® Inc.) using the semi-dry Trans-Blot® SD transfer apparatus (BioRad®, Hercules, CA) for 1 hour at constant current of 0.3 A. RNA was fixed on the membrane by the SpectroLinker™ XL-1500 UV crosslinking apparatus (Spectronics® Corporation, Westbury, NY) using the “optimal crosslink” setting. The membrane was briefly rinsed with water and prehybridized in 50 ml of SXSSC, 0.1% SDS buffer at 45° C. with gentle shaking. Probe hybridization was carried out overnight at 45° C. in 3 ml of prehybridization buffer with gentle shaking. The oligonucleotide probe targeting the hairpin RNA loop was conjugated with TAMRA. Three washes (for 2 minutes each) with 100 ml of water were completed at room temperature and the blot with a ChemiDoc™ MP imaging system (BioRad®, Hercules, CA), using the rhodamine channel.

### C. Preferred Embodiments

The following are among the preferred embodiments of the invention.

One embodiment of the present invention comprises a bacterial host cell containing a plasmid encoding both a gene for the desired dsRNA and a bacteriophage capsid protein gene, such that the dsRNA and the capsid protein genes are transcribed so that the desired dsRNA is produced and the capsid protein gene translated to produce capsid protein and wherein, after a suitable period of time, unencapsidated dsRNA accumulates within the cell to a much higher degree than in the absence of capsid protein. In other embodiments the dsRNA gene and the capsid protein gene may be present on separate compatible plasmids, autonomously maintained phage or other epigenetic elements, or one or both genes may be present within the chromosome of the bacterial host cell.

In an embodiment the dsRNA gene and the capsid protein gene are each transcribed from a transcriptional promoter. The transcriptional promoter may be inducible. In one embodiment the transcriptional promoters are identical; in other embodiments the promoters are different. In still other embodiments the transcriptional promoters may be differentially induced. In such differentially inducible embodiments it may be preferable to induce expression of the capsid protein prior to inducing expression of the dsRNA.

In another embodiment the capsid protein and the dsRNA may be transcribed as a single transcript from a single promoter. The promoter may be inducible. In such embodiments the dsRNA is cleaved from the initial RNA transcript containing the capsid protein coding sequence by post transcriptional processing, such post transcriptional processing may depend on bacterial host cell processes or may be directed by other RNA processing systems such as ribozymes or specific ribonucleases.

In one embodiment one or both of the dsRNA and the capsid protein genes are inducibly transcribed from a transcriptional promoter and transcription is terminated by a transcriptional terminator. In an embodiment the inducible transcriptional promoter is the bacteriophage T7 gene 1 promoter. In other embodiments the inducible transcriptional promoter may be the bacteriophage Lambda PL or PR promoters, the lac operon, tip operon, or synthetic tac promoter, or bacteriophage T5 promoter. Other transcription promoters, both constitutive and inducible, known to those of ordinary skill in the art, may also be used in some embodiments. In an embodiment the transcriptional terminator is the bacteriophage T7 late terminator. Other transcription terminators, both rho-dependent and rho-independent, known to those of ordinary skill in the art may also be used in some embodiments.

In an embodiment the coat protein gene encodes a leviviral capsid protein. The coat protein gene may be the MS2 coat protein gene encoding the MS2 capsid protein or substantially similar sequences retaining the ability to allow accumulation of dsRNA in a microbial host cell. The coat protein gene may encode the Qbeta coat protein gene encoding the Qbeta capsid protein or substantially similar sequences retaining the ability to allow accumulation of dsRNA in a microbial host cell.

In an embodiment the bacterial host comprising the dsRNA is delivered to an insect pest as described herein.

### EXAMPLES

### Example 1

### Unencapsidated dsRNAs are Produced at Higher Levels in the Presence of Capsid Protein than in the Absence of Capsid Protein

Expression strains containing pAPSE10180 and pAPSE10181 were constructed and dsRNA production induced by the standard expression procedure described above. The amount of encapsidated and unencapsidated dsRNA each strain produced was measured as described. The initial impetus for this experiment was to determine whether an RNA molecule with a 180 base pair double-stranded stem structure could be packed within a VLP. A 180 bp dsRNA stem is approximately 60 nm in length, whereas the interior diameter of an MS2 capsid is approximately 20 nm. Based on this geometric limitation, little or no encapsidation was expected and, due to host nuclease activity, little or no unencapsidated dsRNA was expected to be recoverable from the cell lysates. As expected only small amounts of encapsidated dsRNA (en capsid) were recovered (<2 mg/L) from the pAPSE10180 expression cells. In contrast, surprisingly large amounts of unencapsidated dsRNA (ex capsid) were recovered (75-90 mg/L) from the pAPSE10180 expression cells. Even more surprisingly, virtually no unencapsidated dsRNA was recovered from the pAPSE 10181 expression cells.

To determine whether accumulation of RNA is a specific property of the ErkA sequence, or is a more general property of expressing dsRNA in the presence of capsid protein, a series of expression constructs expressing a 294 base sequence from the beta actin gene of the Colorado potato beetle (Leptinotarsa decemlineata strain Freeville, GenBank Accession NM 001300706 between nucleotides 156-335) were produced and tested.

Initially, plasmids expressing the 294 base beta actin sequence from Colorado potato beetle in the sense and the anti-sense orientation were constructed from pAPSE10180 by replacing the ErkA sequences, to produce pAPSE10189 (SEQ ID NO: 4 and pAPSE10190 (SEQ ID NO: 5) respectively. The beta actin sense and antisense strand sequences were amplified by PCR (Accuprime Pfx, Invitrogen Cat. No. 12344040, Thermo Fisher Scientific Inc.) from a gBlock template using primers that introduce the AsiSI and Pmel restriction sites at the 5′ and 3′ ends respectively (gBlock template DNA and PCR primers were synthesized by Integrated DNA Technologies, Coralville IA; all restriction endonucleases were from New England BioLabs, Beverly, MA). Restriction digest of pAPSE 10180 and the beta actin sense and antisense PCR fragment with AsiSI and PmeI resulted in DNA fragments that could be ligated together in the desired manner. The pAPSE10180 plasmid backbone lacking the ErkA sequence was gel purified and the sense and antisense beta actin sequences were ligated into the gel purified vector to produce pAPSE 10189 and pAPSE 10190, respectively. When transformed into a suitable expression host, such as HT115(DE3) the cells containing pAPSE10189 produces a ssRNA transcript comprising 294 bases of the sense strand of the beta actin gene flanked by pac sequences as well as co-express MS2 capsid protein, when cultured and induced as described above. Likewise, cells containing pAPSE10190 produces a ssRNA transcript comprising 294 bases of the anti-sense strand of the same region of the beta actin gene flanked by pac sequences as well as co-express MS2 capsid protein when transformed into a suitable expression host, cultured and induced as described. A second set of plasmids, lacking the ability to express MS2 capsid protein were also produced by replacing the ErkA sequences of pAPSE10181 with the sense and anti-sense 294 base fragments of the beta actin gene as described above. These plasmids, pASPE10274 (SEQ ID NO: 6) and pAPSE10275 (SEQ ID NO: 7) respectively, were transformed into HT115(DE3) and cultured and induced as described.

Analysis of un-encapsidated RNA recovered from the cells whether co-expressed with capsid protein (as with pAPSE10189 and pAPSE10190) or not (pAPSE 10274 and pAPSE10275) showed that virtually no ssRNA can be recovered. However, VLPs recovered from pAPSE10189 and pAPSE10190 yield at least 20 mg/L of ssRNA of sense or anti-sense sequence respectively. This confirms that the plasmid expression systems are capable of producing ssRNA and capsid protein as expected.

A dsRNA expression cassette comprising the 294 base Colorado potato beetle beta actin genes was constructed by a process similar to that described for the dsRNA ErkA expression cassette. In this case, the random DNA sequence comprising the loop between the sense and anti-sense strands of the beta actin sequences comprised 166 bases, including the same internal pac site sequence as found in pAPSE10180 and 10181. This beta actin expression cassette was cloned into pAPSE10180 replacing the ErkA related stem loop sequence to form plasmid pAPSE10269 (SEQ ID NO: 8), and into pAPSE10181 to form plasmid pAPSE10306 (SEQ ID NO: 9). The plasmids were transformed into HT115(DE3), cultured, and induced as described. Analysis of the encapsidated dsRNA produced by the cells containing pAPSE10269 strain showed that 2-10 mg/L dsRNA could be recovered from VLPs. However, much higher levels of the beta actin dsRNA could be recovered from the cells containing pAPSE10269 in unencapsidated form (200 mg/L). Strikingly, analysis of the RNA produced by the pAPSE10306 strain showed that in the absence of co-expressed capsid protein only about 3 mg/L of dsRNA could be recovered.

Thus, the high levels of unencapsidated dsRNA are consistent with a model in which such dsRNA are not packaged efficiently, but for some reason appear to be present within cells co-expressing capsid protein with the dsRNA at much higher levels than in cells which lack capsid protein. One model to account for this observation is that binding of capsid protein to the pac sites inhibits degradation by host cell nucleases.

### Example 2

### Specific Pac Site-Capsid Protein Interaction is not Required for High Level Production of dsRNA

To test whether capsid protein bound to pac sites in the dsRNA results in the observed increase in dsRNA production in cells co-expressing capsid protein, perhaps inhibiting endogenous host nuclease degradation of the bound dsRNA, a series of constructs comprising the basic beta actin dsRNA described above were produced with varying numbers and locations of pac sites. Plasmids pAPSE10216 (SEQ ID NO: 10) and pAPSE10305 (SEQ ID NO: 11), are identical to pAPSE10269 and pAPSE10306 respectively, except they lack the internal loop pac site. Plasmids pAPSE10219 (SEQ ID NO: 12) and pAPSE10304 (SEQ ID NO: 13) are identical to pAPSE10217 and pAPSE10306 respectively, except they have only a single pac site located on the 3′ end of the stem of the dsRNA. Plasmids pAPSE10279 (SEQ ID NO: 14) and pAPSE10303 (SEQ ID NO: 15) are identical to pAPSE10216 and pAPSE 10306 except they lack all pac site sequences entirely. Each of these plasmids was transformed into E. coli HT115(DE3), cultured and induced as described. Analysis of the encapsidated RNA recovered from VLPs of each of pAPSE10216 and pAPSE10219 show that 5-20 mg/L of dsRNA is encapsidated. Strikingly, even the strain containing pAPSE10279 entirely lacking pac sites produced 4 mg/L of encapsidated dsRNA, indicating that this level of encapsidation may represent non-specific entrainment of dsRNA present in the cells at the time the capsids were formed. Furthermore, the strain containing pAPSE10216 produced as much as 250 mg/L of unencapsidated dsRNA in the presence of capsid protein. The strains containing pAPSE10219 and pAPSE10279 produced 30-60 mg/L and 65 mg/L of unencapsidated dsRNA, respectively in the presence of capsid protein. All of the strains containing plasmids comprising the expression cassettes without co-expression of capsid protein produced <4 mg/L of dsRNA.

Together, these results indicate that the ability of capsid protein to increase the amount of unencapsidated dsRNA that can be recovered from cell lysates is not dependent on the specific binding of capsid protein to its cognate pac site sequence. Although the highest levels of unencapsidated dsRNA are recovered from constructs containing at least 5′ and 3′ flanking pac sites (approximately 200 mg/L), significant amounts of unencapsidated dsRNA are produced by constructs having only a single 3′ flanking pac site, or lacking pac sites entirely. Cells containing plasmids producing dsRNA lacking pac sites altogether produce significantly higher amounts of dsRNA (65 mg/L) when capsid protein is co-expressed with the dsRNA relative to the cell lines lacking capsid protein altogether (3-4 mg/L). The approximately 16× increase in recoverable dsRNA between cells co-expressing capsid protein and those lacking capsid protein (65 mg/L versus 3-4 mg/L) is much more than the approximately 3×-4× increase due to the presence of pac sites (65 mg/L versus 200-250 mg/L). The effect of capsid protein co-expression appears to involve something other than mere binding to cognate pac site sequences that may (or may not) be present on the dsRNA.

### Example 3

### Loop Size and Structure are Irrelevant to High Level Production of dsRNA

To test what effect, if any, differences in loop sequence might exert on the production of dsRNA in the presence and absence of co-expressed capsid protein, a series of constructs with different lengths of internal non-homologous loop sequences were inserted between each of the 294 base sense and anti-sense beta actin sequences of pAPSE10269.

Plasmids pAPSE10270 (SEQ ID NO: 16), pAPSE10271 (SEQ ID NO: 17), pAPSE10272 (SEQ ID NO: 18) and pAPSE10292 (SEQ ID NO: 19) have non-homologous loop sizes of 116 bases, 136 bases, 156 bases and 166 bases respectively. Each of these loop sequences has very little propensity for any secondary structure as determined by the m-fold structure prediction program (Zucker and Stiegler (1981) Optimal computer folding of large RNA sequences using thermodynamics and auxiliary information Nucl. Acids. Res. 9(1):133-48). In addition, the 139 base loop sequence found associated with the ErkA stem sequences in pAPSE 10180 and having a slightly higher propensity for structural interactions within the loop was also placed between the sense and anti-sense beta actin sequences of pAPSE10269, to form pAPSE10292. Additionally, pAPSE10291 (SEQ ID NO: 20) comprising a 142 base loop sequence with a high degree of propensity for forming secondary structure based on internal homology was synthesized and constructed as described.

Each of the plasmids described in this Example were transformed into E. coli expression strain HT115(DE3), cultured and induced and the amount of encapsidated and unencapsidated dsRNA determined as described. In each case 2-10 mg/L of dsRNA was recovered from the VLPs produced by inducing expression of the plasmid, indicating that loop size or structure had little or no effect on the ability of VLPs to encapsidate the dsRNA. Likewise, expression from each of the plasmids produced between 100 and 200 mg/L unencapsidated dsRNA, indicating that loop size or structure had little or no effect on overall production of unencapsidated dsRNA in the presence of capsid protein.

### Example 4

### Stem Size is Irrelevant to High Level Production of dsRNA

Differences in stem sequence derived from the Drosophila rnelanogaster ErkA gene sequences expressed from pAPSE10180 and the Colorado potato beetle beta actin gene sequences expressed from pAPSE 10269 do not make a significant difference in the ability in expression strains to produce large quantities of unencapsidated dsRNA (75-90 mg/L from pAPSE10180 versus 200 mg/L from pAPSE10269). Nor does the length of the dsRNA stem (180 base pairs in the dsRNA produced from pAPSE10180 and 294 base pairs in dsRNA from pAPSE10269). To more systematically test what affect, if any, differences stem sequence length might exert on the production of dsRNA in the presence and absence of co-expressed capsid protein, a series of expression constructs with different lengths of stem sequences were substituted for each of the 294 base stem forming sense and anti-sense beta actin sequences of pAPSE10269.

Plasmids pAPSE10276 (SEQ ID NO: 21) and pAPSE10277 (SEQ ID NO: 22) encode dsRNA with potential double-stranded stems of 50 and 75 base pairs respectively. The dsRNA expressed by both plasmids comprise 166 bases of non-homologous loop sequence. Although these dsRNA structures are significantly shorter than those in dsRNA from the corresponding ErkA and beta actin constructs, they still exceed the interior diameter of the MS2 VLP.

When transformed into the E. coli expression strain HT115(DE3), cultured and induced as described, pAPSE 10276 produces 5-10 mg/L of encapsidated dsRNA and 80-120 mg/L of unencapsidated dsRNA. Plasmid pAPSE 10277 produces 20-30 mg/L encapsidated dsRNA and 200-250 mg/L unencapsidated dsRNA. These values are similar to those observed for pAPSE10180 and pAPSE10269 described earlier in this Example, indicating that differences in stem length and sequence do not play a major role in producing dsRNA in cells co-expressing capsid protein.

### Example 5

### Capsid Protein is Required for High Level Production of dsRNA

To confirm the requirement for capsid protein, plasmid pAPSE10216, which produces a dsRNA product at high levels in the presence of capsid protein, was altered to replace the MS2 coat protein gene with eGFP. A gBlock template comprising the T7 promoter to T7 terminator sequences of pAPSE10216 (spanning the sequences between the unique BarHl and Sall sites of the plasmid) in which the coding sequence of MS2 coat protein was replaced with the coding sequence of eGFP was designed, produced and amplified with primers encompassing the BamHl site on the 5′ side and the Sall site on the 3′ side. The resulting 1 kb fragment was digested with BamHl and Sall and then ligated into BamHl-Sall digested pAPSE10216 to form pAPSE10366 (SEQ ID NO: 24). Plasmid pAPSE10366 was confirmed by restriction digest and transformed into the E. coli expression strain HT115(DE3), cultured and induced as described, pAPSE 10366 produces <2 mg/L of unencapsidated dsRNA, in contrast to the 200 mg/L produced by pAPSE10216. In addition, the cells expressed high amounts of eGFP as evidenced by the intense fluorescence produced on induction (data not shown) confirming that the basic dual expression plasmid used throughout these studies performs as expected. This result further demonstrates that capsid protein is necessary for accumulation of unencapsidated dsRNA in cells expressing the target RNA gene that otherwise accumulate unencapsidated dsRNA in the presence of capsid protein.

To further confirm that the presence of capsid protein is essential to the high levels of unencapsidated dsRNA production a plasmid compatible with pAPSE 10181 and capable of inducible expression of the MS2 capsid protein is constructed. pAPSE10149 (SEQ ID NO: 23) is based on pACYC184. This plasmid comprises a PISA origin of replication that is not excluded by the colE1 based origin of replication of pAPSE10181 and a chloramphenicol acetyl transferase antibiotic marker to allow selection of co-transformants containing both pAPSE10181 (encoding ampicilin resistance) and pAPSE10149 (encoding chloramphenicol resistance). Plasmid pAPSE10149 also comprises the same T7 promoter sequence capable of driving transcription of a single copy of the bacteriophage MS2 capsid gene followed by a T7 terminator as found in pAPSE10118 cloned into the BamHI and Sphl sites of pACYC184. Plasmid pAPSE10149 is transformed into expression strains already containing pAPSE10181 to produce ampicilin and chloramphenicol resistant double transformants. Expression studies of such double transformants show that co-expression of the capsid protein from pAPSE10149 in conjunction with pAPSE10181 produces 200 mg/L of unencapsidated dsRNA whereas cells containing pAPSE10181 alone produce <2 mg/L of unencapsidated dsRNA (see Example 1). This demonstrates that providing capsid protein in trans is sufficient to facilitate production of high levels of unencapsidated dsRNA to host cells containing a plasmid expressing the dsRNA target that otherwise fail to accumulate unencapsidated dsRNA in the absence of capsid protein.

### Example 6

### Other Capsid Proteins can Induce High Level Production of dsRNA

To test whether the accumulation of unencapsidated dsRNA is a unique property of bacteriophage MS2 capsid protein, or whether other capsid proteins share this property, a plasmid expression system analogous to pAPSE10216 was constructed. This plasmid, pAPSE10359 (SEQ ID NO: 25) comprises a Qbeta capsid protein and Qbeta pac sites at the 5′ and 3′ ends of the beta actin dsRNA expression cassette, but is in all other aspects similar to pAPSE10216.

Briefly, the Qbeta coat protein gene sequence (Genebank Accession NC_001890 between nucleotides 1343 and 1744) was synthesized as a gBlock fragment by Integrated DNA Technologies, Coralville, IA. The synthetic fragment was amplified with PCR with primers that introduced a BamHI restriction site followed by a T7 promoter sequence upstream of the Qbeta coat protein gene followed by a T7 terminator and a Sphl restriction site. The amplified synthetic fragment and plasmid pBR322 were digested with BamHI and Sphl and ligated together to form intermediate plasmid pAPSE10358. The beta actin dsRNA sequence of pAPSE10269 was amplified by PCR with primers that introduced an EcoRI restriction site followed by a Qbeta pac sequence followed by the beta actin dsRNA sequence followed by a second copy of the Qbeta pac sequence followed by a BamHI restriction site. This amplified beta actin containing sequence and plasmid pAPSE10358 were digested with EcoRI and BamHl and ligated together to form pAPSE10374. Plasmids pAPSE10374 and pAPSE10216 were digested with AsiSI and NotI. This cleaves pAPSE10374 into two fragments of 4,713 and 113 base pairs and pAPSE10216 into two fragments of 5,204 and 786 base pairs. The 4,713 and 786 base pair fragments were isolated and ligated together to produce pAPSE10359.

When transformed into the E. coli expression strain HT115(DE3), cultured and induced as described, pAPSE10359 will produce a large amount of unencapsidated dsRNA relative to the amount of dsRNA produced from a similar construct lacking capsid protein (pAPSE10305). This pattern, similar to that observed for pAPSE10216 and pAPSE10305 described in Example 1, will confirm that expression of the Qbeta capsid protein, like the MS2 capsid protein, is sufficient to increase the amount of dsRNA produced in vivo.

### Example 7

### RNA Binding Proteins Other than Capsid Proteins are not Sufficient for High Level Production of dsRNA

To test whether the accumulation of unencapsidated dsRNA is a function of general RNA binding or is specific to bacteriophage capsid proteins, a plasmid expression system, pAPSE10357 (SEQ ID NO: 26) was constructed comprising the RNA binding domain of the human U1A protein and its hairpin cognate binding site from human Ul snRNA 5′ and 3′ of the sense and antisense stem loop structure of the beta actin dsRNA. Plasmid pAPSE10357 is similar to pAPSE10216 with the capsid protein replaced by the human U1A RNA binding protein and U1A binding site sequences at the 5′ and 3′ ends of the beta actin dsRNA expression cassette, but is in all other aspects similar to pAPSE10216.

The DNA sequence encoding the N-terminal 102 amino acids comprising the RNA binding domain of the human U1A protein was amplified from a cloned copy of the U1A protein (Plasmid pAV105, Professor Kathleen Hall, Washington University, St. Louis, MO) using PCR primers that introduced a BamHI restriction site followed by a T7 promoter sequence upstream of the U1A gene fragment followed by a T7 terminator and a Sphl restriction site. The amplified synthetic fragment and plasmid pBR322 were digested with BamHI and Sphl and ligated together to form intermediate plasmid pAPSE10356. The beta actin dsRNA sequence of pAPSE10269 was amplified by PCR with primers that introduced an EcoRI restriction site followed by the hairpin binding site sequence from human Ul snRNA sequence followed by the beta actin dsRNA sequence followed by a second copy of the hairpin binding site sequence from human Ul snRNA sequence followed by a BamHI restriction site. This amplified beta actin containing sequence and plasmid pAPSE10356 were digested with EcoRI and BamHI and ligated together to form pAPSE10373. Plasmids pAPSE10373 and pAPSE10216 were digested with AsiSl and Notl. This cleaves pAPSE10373 into two fragments of 4,627 and 113 base pairs and pAPSE10216 into two fragments of 5,204 and 786 base pairs. The 4,713 and 786 base pair fragments were isolated and ligated together to produce pAPSE10357.

When transformed into the E. coli expression strain HT115(DE3), cultured and induced as described, pAPSE10357 will not produce a significant amount of unencapsidated dsRNA relative to the amount of dsRNA produced from a similar construct lacking capsid protein (pAPSE10305). This will confirm that the mere presence of an RNA binding site and binding protein in conjunction with the dsRNA is not sufficient to increase the amount of dsRNA produced in vivo. Alternatively, production of significant amounts of unencapsidated dsRNA will indicate that the presence of RNA binding sites at the 5′ and 3′ end and the cognate RNA binding protein is sufficient for increasing in vivo production of dsRNA.

### Example 8

### The N-Terminus of Capsid Protein is Sufficient for High Level Production of dsRNA

To examine whether the increased production of dsRNA from plasmids containing both the dsRNA gene and the coat protein gene requires the intact capsid protein or whether only a portion of the protein is required, a frame-shift mutation was introduced into the coat protein gene sequence of pAPSE10180. Double digestion of pAPSE10180 with the restriction enzymes Stul and PmlI produces two restriction fragments, a large fragment of 5,485 base pairs and a small thirteen base pair fragment comprising about 4 codons of the capsid protein CDS about 40 codons from the coat protein start codon of pAPSE10180. The restriction enzymes produce blunt-ended termini and the larger fragment was re-ligated to produce plasmid pAPSE10372 (SEQ ID NO: 27), which, in addition to producing an intact inducible dsRNA ErkA-specific sequence, also comprises an inducible frame-shifted protein that includes the N-term 41 codons of the MS2 coat protein followed by 27 codons of frame-shifted sequence before terminating at a stop codon (SEQ ID NO: 28). When pAPSE 10372 was transformed into E. coli expression strain HTE115(DE3) and cultured and induced as described, 75 mg/L of dsRNA was produced. This indicates that the N terminus of the capsid protein alone is sufficient to increase production of dsRNA as well as the intact capsid protein (compare yields from pAPSE10180 and pAPSE 10372 in Table 1).

The N-terminus of the MS2 capsid protein forms a distinctive three-dimensional structure comprised of four separate beta sheets (D. Peabody, The RNA binding site of bacteriophage MS2 coat protein, The EMBO Journal 12(2) 595-600 (1993)). Each of these sheets, 3D from amino acids 31-35, (3C from amino acids 2225, 13B from amino acids 19-21 and OA amino acids 8-11 may play a role in the ability of the N-terminus capsid protein fragment to improve dsRNA production. Note that the nomenclature is that of Peabody and the numbering includes the N-terminal methionine omitted by Peabody. Progressive deletion of each of these structural motifs can determine the minimum sequence requirement for improving dsRNA production.

### Example 9

### Fed Batch Fermentation Produces Very High Level Production of dsRNA

To determine whether quantities of dsRNA could be increased by improving the microbial growth conditions, glucose fed batch fermentations were conducted. Briefly, fed-batch fermentations were carried out in an Eppendorf BioFlo 115 fermenter at 37° C. The pH was controlled by automatic addition of 30% ammonium hydroxide. The dissolved oxygen probe was calibrated to 0% by unplugging the DO probe and to 100% with air saturation. The vessel was aerated at 2 vvm and dissolved oxygen maintained at 30% by cascade control of agitation. An overnight culture of HT115 (DE3) containing pAPSE10379 was grown in LB containing 100 ug/ul of ampicillin and 12.5 ug/ul of tetracycline at 37° C. to inoculate the seed medium. The seed media is a defined media consisting of 5.68 g/L Na2HPO4, 1.34 g/L KH2PO4, 6.6 g/L (NH4)2SO4, 10 g/L glucose, 1× trace metal and 1× vitamin solutions maintained at a pH of 7.0. To ensure plasmid stability antibiotics are added at 100 ug/ul ampicillin and 12.5 ug/ul tetracycline. At saturation (OD600 3-5) the seed cultures are used to provide 10% inoculum for the fermenter.

During fed batch-cultures a 50% (w/v) solution of glucose was added according to a carbon limiting DO stat feeding strategy. The basal medium consists of 6 g/L K2HPO4, 3 g/L NaHPO4, 10 g/L (NH4)2SO4, 1 g/L MgSO4, 1× trace metal solution with antibiotics added at 100 ug/ul of ampicillin and 12.5 ug/ul of tetracycline. Upon exhaustion of the initial carbon source provided by the glucose the feed solution is added automatically in a manner that maintains the DO level at 30% of saturation.

Once the cell culture has reached an OD600 of 60 the cells are induced with 1 mM IPTG or a feed of 20 g/L of lactose by switching the glucose feed to a lactose feed. After induction 1 mL samples are taken at different times post induction. The samples are lysed by sonication of the cell pellet into 20 mM Tris-HCl at pH 7. Total RNA from the cell pellet is purified using well-known Trizol extraction procedures. Briefly 1 volume of cell lysate is added to 1 volumes of Trizol RNA extraction reagent. Addition of 1 volume of chloroform results in the RNA partitioning to the aqueous layer leaving the protein and DNA contaminants behind.

To analyze the yield of dsRNA the total RNA sample is diluted to 1 ug/ul and subjected to RNAseA treatment. The reaction is carried out in 20 mM Tris at pH 7.0 and 37° C. for 40 minutes. Once this is done proteinase K is added to the reaction to remove the nuclease and is allowed to react at 37° C. for 40 minutes. Upon completion of this step the dsRNA remaining is diluted in half, quarters and eighths in order to determine the concentration of the dsRNA using gel densitometry.

Quantification of dsRNA yield by gel densitometry was performed by comparing the intensity of dsRNA bands versus dsDNA bands of known mass and weight on a 1.5% agarose gel containing ethidium bromide. The lambda 100 bp quantifiable DNA marker was used and a standard curve was generated to determine the range in which the dsRNA from the fermentation can be reliably quantified. The computer program calculates the amount of dsRNA in the amount of sample loaded on the gel and a back calculation that considers the dilution steps is performed. Yields of dsRNA at levels as high as 3 g/L have been calculated with both IPTG and lactose as inducers under these conditions. These results indicate that further increases in dsRNA production are possible by improving fermentation conditions.

### Example 10

### Compositions and Methods for dsRNA Production in Tram Positive Bacteria

The ability of gram-positive bacteria to produce increased levels of dsRNA by co-expression of capsid proteins can be examined in the following manner. Corynebacterium glutamicum MB001(DE3) strain DSM 102071, containing an inducible T7 RNA polymerase gene (described in Kortmann, et al., A chromosomally encoded T7 RNA polymerase-dependent gene expression systemfor Corynebacterium glutamicum; construction and comparative evaluation at the single cell level. Microb Technol. 8(2):253-65. March 2015) is modified to knockout the me gene homolog encoding RNAse III. Briefly, PCR primers capable of amplifying a 1.2 kb sequence homologous to the sequence present in C. glutamicum strain MB001(DE3) immediately upstream of the me gene and PCR primers capable of amplifying a 1.5 kb sequence homologous to the sequence immediately downstream of the me gene are synthesized. A PCR amplification reaction using C. glutamicum strain MB001(DE3) genomic DNA and said primers results in a single DNA fragment comprising the 1.2 kb and 1.5 kb target sequences joined together (by standard overlap PCR methods) to produce an approximately 2.7 kb SalI-BamHI synthetic DNA fragment. This SalI-BamHI DNA fragment and plasmid pK18mobsacB (ATCC 87097, described by Schafer, et al., Small mobilizable multipurpose cloning vectors derivedfrom the Escherichia coli plasmids pK18 and pK19: selection of defined deletions in the chromosome of Corynebacterium glutamicum. Gene 145:69-73) are digested with Sall and BamHI and the products ligated together to produce plasmid pAPSE10429 (SEQ ID NO: 29). Plasmid pAPSE 10429 is transformed into C. glutamicum strain MB001 and transformants selected on kanamycin containing solid LB medium to identify chromosomal integrants. Kanamycin resistant clones are transferred to a solid LB medium containing 20% sucrose. Conversion of sucrose by the sacB gene product is toxic to C. glutamicum strain MB001 so only those chromosomal integrants that subsequently delete the sacB gene from the chromosome can survive on such media. Surviving colonies are grown up and screened by PCR to confirm concomitant loss of the rnc locus from the chromosome. The desired strain is designated C. glutamicum MB001(DE3) rnc. This strain possesses an inducible T7 RNA polymerase and lacks the rnc gene and is suitable for testing the efficacy of dsRNA production in the presence and absence of capsid protein.

A shuttle vector capable of expression of capsid coat protein and dsRNA in both E. coli and C. glutamicum is constructed by synthesizing a DNA comprising the origin of replication of the gram-positive plasmid pCG1 (GeneBank Accession No. AB027714; described by Trautwetter and Blanco, Structural organization of the Corynebacterium glutamicumplasmid pCG100. J. Gen. Microbiol. 137:2093-101 1991) and the kanamycin resistance gene of pK18mobsacB. This synthetic DNA (SEQ ID NO: 30) is ligated into the previously described dsRNA containing plasmids at the unique NruI restriction site to allow testing whether the presence of capsid protein in gram-positive C. glutamicum MB001(DE3) rnc strain produces dsRNA at high levels as described below.

Insertion of the synthetic DNA comprising the pCG1 origin of replication and the kanamycin resistance gene is accomplished by digesting pAPSE10279 with Nrul and ligating the phosphorylated synthetic DNA into the plasmid to produce plasmid pAPSE10430 (SEQ ID NO: 31). Plasmid pAPSE10430 contains the kanamycin resistance gene, the bacteriophage MS2 coat protein, and the dsRNA construct based on the previously described 294 base sense and antisense sequences homologous to the Colorado potato beetle beta actin gene separated by a 166 base non-homologous loop and entirely lacking any pac sequences. In similar fashion, the synthetic DNA comprising the pCG1 origin of replication and the kanamycin resistance gene is also ligated into Nrul digested pAPSE10303 to produce pAPSE10431 (SEQ ID NO: 32). Plasmid pAPSE10431 contains resistance genes to ampicillin and kanamycin, as well as the same inducible dsRNA construct as pAPSE10430. However, pAPSE10431 lacks the inducible MS2 coat protein gene of pAPSE10430. The relevant features of pAPSE10430 and pAPSE10431 are presented in Table 2 and the relationship between these two plasmids and their parental plasmids, pAPSE 10279 and pAPSE10303, respectively, can be determined by comparing Table 2 and Table 1.

Additional plasmids containing one, two, and three pac sites, with and without MS2 coat protein, are constructed using the same procedure. Plasmid pAPSE10432 (SEQ ID NO: 33) containing a single pac site 3′ of the beta actin stem loop structure and encoding the MS2 coat protein gene is produced by ligating the synthetic DNA fragment into the Nrul site of pAPSE10219. Plasmid pAPSE10433 (SEQ ID NO: 34) is produced by ligating the synthetic DNA fragment into the Nrul site of pAPSE10304. Plasmid pAPSE10433 is identical to pAPSE10432 except it lacks an inducible MS2 coat protein gene. Plasmid pAPSE10434 (SEQ ID NO: 35) containing two pac site sequences located one on either side of the beta actin stem loop and encoding the MS2 coat protein is produced by ligating the synthetic DNA fragment into the Nrul site of pAPSE10216. Plasmid pAPSE10435 (SEQ ID NO: 36) is produced by ligating the synthetic DNA fragment into the Nrul site of pAPSE10305. Plasmid pAPSE10435 is identical to pAPSE10434 except it lacks an inducible MS2 coat protein gene. Plasmid pAPSE10436 (SEQ ID NO: 37) containing three pac site sequences with one each 5′ and 3′ of the beta actin stem loop and one within the loop sequence itself (as depicted in FIG. 1) and encoding the MS2 coat protein is produced by ligating the synthetic DNA fragment into the Nrul site of pAPSE10269. Plasmid pAPSE10437 (SEQ ID NO: 38) is produced by ligating the synthetic DNA fragment into the Nrul site of pAPSE10306. Plasmid pAPSE10437 is identical to pAPSE104360 except it lacks an inducible MS2 coat protein gene.

In each case, following ligation of the synthetic DNA fragment into the Nrul site of the target plasmid, transformants the ligation reactions are desalted and transformed in to C. glutamicum MB001(DE3)rnc and selected for resistance to kanamycin. The selected clones are subsequently grown at 32° C. in 100 ml of LB media containing kanamycin until the culture reaches OD600 0.8, at which time isopropyl I3-D-thiogalactopyranoside is added to a final concentration of 1 mM to induce T7 polymerase directed transcription of the MS2 coat protein and the dsRNA, or just the dsRNA precursor in the plasmids lacking coat protein. The induced cultures are allowed to grow for at least 4 hours post-induction to allow sufficient time for accumulation of the MS2 coat protein and dsRNA target. Cells are collected by centrifugation at 3,000 g at 4 C. Each pellet is stored at 4° C. until processing.

The dsRNA is purified by re-suspending each pellet in approximately 0.1 volume of 20 mM Tris-HCl, pH 7.0, containing 10 mM NaCl and sonicated to lyse the cells. Cell debris is removed by centrifugation at 16,000 g. The resulting lysate is mixed with 3 volumes of Trizol (Ambion Life Technologies) and the RNA is extracted by adding 1 volume of chloroform. Addition of NaCl to a final concentration of 500 mM to the aqueous layer and subsequent ethanol precipitation results in a pellet containing the 294 bp siRNA precursor and RNA from the C. glutamicum host.

To determine the amount of dsRNA produced by the C. glutamicum transformed with plasmids containing various pac site configurations, with and without MS2 coat protein, the ethanol pellets are resuspended and treated with RNAseA for 1 hour at 37° C. followed by Proteinase K digestion for 1 hour at 37° C. Quantification of the dsRNA is accomplished by gel densitometry using a BioRad ChemiDoc MP Imaging System. Several dilutions of the treated dsRNA are run on a 1.5% agarose gel containing 0.001% ethidium bromide. A 100 bp quantifiable dsDNA ladder (QuantiBP DNA ladder Lambda) is used as the standard curve and the dsRNA is quantified at the concentration that falls within the linear range of the standard curve. Software such as Image Lab 4.1 determines the concentration of the dsRNA loaded on the gel and a final yield of dsRNA is determined by accounting for the dilutions associated with the dsRNA samples present on the gel.

Table 2 summarizes the predicted results of the dsRNA yield determination of the Colorado potato beetle beta actin dsRNA produced by C. glutamicum MB001(DE3) me and the various plasmids described above. Such results confirm that gram positive hosts such as C. glutamicum produce large quantities of dsRNA by co-expression of the MS2 coat gene and a dsRNA target of interest.

TABLE 2
Predicted production of dsRNA by C. glutamicum MB001(DE3) me as a function of
variation in dsRNA structure and the presence or absence of coat protein.

| | | Loop | Stem | | | |
|---|---|---|---|---|---|---|
| RNA Structure | | size | size | Stem | Coat | dsRNA |
| as depicted in | Plasmid | (bases) | (bp) | sequence | protein | (mg/L) |
| FIG. 6 | pAPSE10430 | 166 | 294 | beta actin | MS2 | ~60 |
| FIG. 6 | pAPSE10431 | 166 | 294 | beta actin | none | ~4 |
| FIG. 5 | pAPSE10432 | 166 | 294 | beta actin | MS2 | ~120 |
| FIG. 5 | pAPSE10433 | 166 | 294 | beta actin | none | ~4 |
| FIG. 4 | pAPSE10434 | 166 | 294 | beta actin | MS2 | ~250 |
| FIG. 4 | pAPSE10435 | 166 | 294 | beta actin | none | ~4 |
| FIG. 1 | pAPSE10436 | 166 | 294 | beta actin | MS2 | ~250 |
| FIG. 1 | pAPSE10437 | 166 | 294 | beta actin | none | 4 |

### Example 11

### Compositions and Methods for dsRNA Production in Yeast

To create a Saccharomyces cerevisiae production host suitable for dsRNA accumulation utilizing the MS2 bacteriophage coat protein, the Rntl gene of S cerevisiae YPH 500 (ATCC 7662.6) is knocked out according to the procedure of Gardenr and Jasperson (Gardner, J M and Jaspersen, S L, Manipulating the yeast genome: deletion, mutation and tagging by PCR. Methods Mol Biol. 1205:45-78, 2014). The KanMx4 gene is amplified from pML104-KanMx4 plasmid (Laughery, et al., New vectors for simple and streamlined CRISPR-Cas9 genome editing in Saccharomyces cerevisiae. Yeast 32(12):711-20 Sep. 21, 2015) with PCR primers including 60 base pair (bp) upstream (forward primer) and 60 bp downstream (reverse primer) regions of the S. cerevisiae Rntl gene. The resulting PCR product is introduced into chemically competent S. cerevisiae cells following the established S. cerevisiae transformation protocol. The transformed cells are incubated overnight without selection marker to allow for homologous recombination to occur, where in the kanMx4 gene carrying 60 bp upstream and downstream regions of Rntl replaced the Rntl gene. Following overnight incubation, the transformed cells are plated on YPD plates carrying G418 as selection marker. G418 resistant colonies are screened by PCR to confirm presence of kanMx4 gene and deletion of Rntl gene in the YPH 500 genome.

S. cerevisiae expression vectors pESC-His, pESC-Leu, pESC-Ura and pESC-Trp are widely used for recombinant protein expression in S. cerevisiae. Each of the pESC vectors (Agilent Technologies, Santa Clara CA) contains one of four different yeast-selectable markers (HIS3, TRP1, LEU2, or URA3) in the same vector backbone, which allows expression of two different genes in a single yeast cell. The pESC series vectors are used with S. cerevisiae strain YPH 500 (M ATaura3-52 lys2-801 amber ade2-101 ochre trpl-A63 his3-A200 leu2-A1). In this example, the pESC-Trp vector is selected for expression of MS2 coat protein and target dsRNA sequence inside S. cerevisiae, although any of the other pESC vectors could be employed using similar methods since these vectors can replicate in S. cerevisiae as well as E. coli, which facilitates molecular manipulations necessary to produce dsRNA.

The pESC-Trp vector is modified by cloning a 50-base pair multi-cloning site linker containing BamHI, Swat, AsiSI, Notl, SacII and Nhel sites, downstream of the GAL1 promoter into the existing BamHI and Nhel sites. Following this, the beta actin stem loop sequence (dsRNA) of pAPSE10279 is excised as an AsiSl/Notl fragment and ligated into the AsiSl/Notl sites of the modified pESC-Trp vector. Expression of the dsRNA in this plasmid is under the control of galactose inducible promoter GALL. The new vector is named pAPSE10439 (SEQ ID NO: 39). Another plasmid, pAPSE10440 (SEQ ID NO: 40), which is identical to pAPSE10439, but also includes the MS2 coat protein. Plasmid pAPSE10440 is constructed by PCR amplifying the MS2 coat protein expression sequences of pAPSE10279 with a forward primer carrying an EcoRI restriction site on the 5′ end and the reverse primer carrying Sacl site on the 3′ end. The PCR product is digested with EcoRI and Sacl and cloned into the cognate sites of pAPSE10439. Thus, pAPSE10439 inducibly expresses the dsRNA from the GAL1 promoter, whereas pAPSE 10440 inducibly expresses the dsRNA sequence from the GAL1 promoter and the MS2 coat protein from the GAL10 promoter,

Similar plasmid pairs are constructed using this technique. Plasmids pAPSE10441 (SEQ ID NO: 41) and pAPSE10442 (SEQ ID NO: 42) are produced by digesting pAPSE10439 and pAPSE10440 with AsiSI and Noll and isolating the vector fragment. Plasmid pAPSE10219 is also digested with AsiSI and Notl and the dsRNA sequence is isolated. The isolated dsRNA sequence is ligated into the pAPSE10439 vector to form pAPSE10441 and the isolated dsRNA sequence is ligated into the pAPSE10440 vector to form pAPSE10442. Plasmids pAPSE10443 (SEQ ID NO: 43) and pAPSE10444 (SEQ ID NO: 44) are produced by digesting pAPSE10439 and pAPSE10440 with AsiSI and Notl and isolating the vector fragment. Plasmid pAPSE10216 is also digested with AsiSI and Noll and the dsRNA sequence is isolated. The isolated dsRNA sequence is ligated into the pAPSE10439 vector to form pAPSE10443 and the isolated dsRNA sequence is ligated into the pAPSE10440 vector to form pAPSE10444. Plasmids pAPSE10445 (SEQ ID NO: 45) and pAPSE10446 (SEQ ID NO: 46) are produced by digesting pAPSE10439 and pAPSE 10440 with AsiSI and Notl and isolating the vector fragment. Plasmid pAPSE 10269 is also digested with AsiSI and Notl and the dsRNA sequence is isolated. The isolated dsRNA sequence is ligated into the pAPSE10439 vector to form pAPSE10445 and the isolated dsRNA sequence is ligated into the pAPSE10440 vector to form pAPSE10446.

Chemically competent YPH 500 DRnt1 cells are transformed with each of the above mentioned plasmids (pAPSE10439-46) separately and individual clones selected on synthetic dextrose minimal (SD) tryptophan (trp) drop out plates. After inoculating the 100 ml SD-Trp drop out broth the cultures are grown for 12 to 16 hours. The cells from the culture are then harvested by centrifugation at 3000 g for 5 minutes, the cell pellet is washed once with sterile water and the cells re-suspended in synthetic galactose minimal broth (SG) lacking tryptophan. The cells are grown in the SG-trp drop out broth overnight to induce production and accumulation of dsRNA and MS2 coat protein (where appropriate). Cells are harvested by centrifugation at 3,000 g at 4 C. Each pellet is stored at −20° C. until processing.

The dsRNA is purified by re-suspending each pellet (10 ml culture) in approximately 1.0 ml of yeast cell lysis buffer (Sigma C4482). The resulting lysate is mixed with 3 volumes of Trizol (Ambion Life Technologies) and the RNA extracted by adding 1 volume of chloroform. Addition of NaCl to a final concentration of 500 mM to the aqueous layer and subsequent ethanol precipitation results in a pellet containing the dsRNA and RNA from the S. cerevisiae host. The resulting RNA pellet is dissolved in 20 mM Tris HCl pH 7.0 and RNA concentration of the sample determined. To determine the amount of dsRNA produced by the S. cerevisiae strains, a known amount of RNA (10 ug) from each RNA sample from pAPSE10439-pAPSE10446) are digested with RNAseA for 1 hour at 37° C. followed by Proteinase K digestion for 1 hour at 37° C. The resulting samples contain only the dsRNA target. Quantification of the dsRNA is done by gel densitometry using a BioRad ChemiDoc MP Imaging System. Several dilutions of the RNAse A reaction are run on a gel that contains 1.5% agarose and 0.001% ethidium bromide. A 100 bp quantifiable dsDNA ladder (QuantiBP DNA ladder Lambda) is used as the standard curve and the dsRNA is quantified at the concentration that falls within the linear range of the standard curve. Using Image Lab 4.1 software, the concentration of the dsRNA loaded on the gel is determine and a final yield of dsRNA calculated by accounting for the dilutions of the dsRNA loaded on the gel.

Table 2 summarizes the predicted results of the dsRNA yield determination of the Colorado potato beetle beta actin dsRNA produced by S. cerevisiae YPH-500 and the various plasmids described above. Such results confirm that yeasts such as S. cerevisiae produce large quantities of dsRNA by co-expression of the MS2 coat gene and a dsRNA target of interest.

TABLE 2
Predicted production of dsRNA by S. cerevisiae
YPH 500 as a function of variation in dsRNA
structure and the presence or absence of coat protein.

| RNA | | | | | | |
|---|---|---|---|---|---|---|
| Structure | | Loop | Stem | | | |
| as de- | | size | size | Stem | Coat | dsRNA |
| picted in | Plasmid | (bases) | (bp) | sequence | protein | (mg/L) |
| FIG. 6 | pAPSE 10440 | 166 | 294 | beta actin | MS2 | −60 |
| FIG. 6 | pAPSE 10439 | 166 | 294 | beta actin | none | −4 |
| FIG. 5 | pAPSE 10442 | 166 | 294 | beta actin | MS2 | −120 |
| FIG. 5 | pAPSE 10441 | 166 | 294 | beta actin | none | −4 |
| FIG. 4 | pAPSE 10444 | 166 | 294 | beta actin | MS2 | −250 |
| FIG. 4 | pAPSE 10443 | 166 | 294 | beta actin | none | −4 |
| FIG. 1 | pAPSE 10446 | 166 | 294 | beta actin | MS2 | −250 |
| FIG. 1 | pAPSE 10445 | 166 | 294 | beta actin | none | 4 |

### Example 12

### Delivery of dsRNA to Colorado Potato Beetle Larvae

The superior environmental stability and delivery of dsRNA according to the present invention is demonstrated by delivering naked dsRNA targeting a gene of interest or Corynebacterium glutamicum comprising dsRNA targeting a gene of interest (“test substances”) to mixed sex, seven-day old Colorado potato beetle larvae of similar size. The test substances are provided as a solution and contain a silicone surfactant to enhance spreading and prevent roll off form the leaf surface.

Briefly, a 10 μl droplet of each test substance is added to the surface of a potato leaf disc. The solution is spread with the pipette tip to cover at least the central half of the leaf disc, with the assumption that the larvae will devour all of the leaf tissue without veins. The pipette is discarded after applying test solution to a leaf disc to prevent the leaf's RNAse's from contaminating the test solution.

The test substance is allowed to dry on the leaf surface prior to test initiation. At day 0, the CPB larvae is starved for 12 hours and then placed on to the treated leaf disc in the petri dish. The larvae is allowed to feed on the treated leaf disc until leaf tissue is devoured (only leaf veins are remaining).

Once the treated leaf discs are completely consumed, the remaining veins are removed and discarded and the larvae is placed on a maintenance diet of potato leaves. Following treatment, larvae is maintained for 15 days on the maintenance diet.

During the test period the test individuals are observed daily to record mortality. The test is preferably carried out with 4 replications with 5 larvae/replication.

### Example 13

### Delivery of dsRNA to Diamond Back Moths

The test is preferably conducted with four replications and includes appropriate controls, including a non-specific RNA control.

Second-instar larvae of diamond back moths are used for the test. Test individuals are starved for 12 hours prior to testing. Each test substance (“naked” dsRNA” targeting inhibitor of apoptosis gene or Corynebacterium glutamicum comprising dsRNA targeting inhibitor of apoptosis gene) will contain a silicone surfactant to enhance spreading and prevent roll off from the leaf surface. Each application to a 1 cm² leaf disc will be made in 10 ml volume. Tubes containing each test substance is vortexed prior to use.

After application of each test substance to the leaf disc, the disposable pipette tip is used, if necessary, to help spreading of the liquid on the leaf surface. A new pipette tip is used for each application to prevent RNAses from the leaf surface destroying the RNA in the treatment solutions.

The test substances are allowed to dry on the leaf surface prior to initiating the test. The treated cabbage leaf disc is placed in a petri dish (each petri dish serves as a replication for a treatment). DBM larvae (10 second instar larvae replication) is applied to the dried leaf surface. After allowing the larvae to feed on the treated leaf discs for 48 hours (or before, if all of the leaf disc has been consumed), larvae is shifted to untreated leaves to observe larvae survival/mortality. The larvae are considered alive if they move in response to touch during the observation period. During the test, the temperature should be maintained at 26 C.

A significant increase in mortality is demonstrated with the Corynebacteriurn glutamicum comprising dsRNA compared to the “naked” dsRNA.

### Example 14

### Environmental Stability of dsRNA in Corynebacterium glutamicum

The environmental stability of dsRNA in Corynebacteriurn glutamicum was assessed in several pertinent environments. Briefly, Corynebacteriurn glutamicum comprising an expression cassette encoding a dsRNA of about 350 base pairs of inhibitor of apoptosis gene in Diamondback moths, were incubated under appropriate conditions to produce a significant amount of dsRNA.

Briefly, 650 μl of concentrated host cells carrying the target dsRNA were centrifuged and the supernatant (around 350 μ.1) was removed and replaced with 350 μl of tap water, pond water or puddle water (“rain water collected in a pot hole”) in separate tubes. The tubes were then incubated at room temperature for the following time periods: (i) tap water for 168 hours (ii) pond water for 72, 96 or 168 hours and (iii) puddle water for 72, 96 or 168 hours. The amount of dsRNA in each case was assessed by collecting 100 μl of host cells at the specified time periods and extracting dsRNA. 500 ng of total RNA (RNaseA/ProtK treated), as assessed by nanodrop reading, were loaded on a gel for each sample and the amount of target dsRNA was quantified. The amount of dsRNA at 0 hours (i.e. the starting amount of dsRNA) served as a control.

For “naked” dsRNA, in each case, 1.0 μg of dsRNA in 5 μl+95 μl water of puddle, pond or tap water were incubated at room temperature. Aliquots were collected at the specified time points and assessed on gel as described above.

As shown in Table 3 below, after 7 days of incubation at room temperature in water from different sources, about 64% of the target dsRNA was recovered from the host cells. In stark contrast, substantially all of the “naked” dsRNA was degraded over the same time periods in the identical environments. The ability of Corynebacterium glutamicum cells to protect dsRNA for more than 7 days in pond water (which contains various microbes and RNAses) indicates that Corynebacterium glutamicum can be used to deliver target dsRNA to insect pests in particularly relevant environments for insect pest control, such as puddle water, pond water and a leaf surface. Further, this experimental data is very strong evidence for the use of the present methods to stably delivery dsRNA to e.g. mosquito larvae in standing water.

Notably, some initial degradation of dsRNA within the C. glutamicum host occurs within the first 24 hours, which seems to be due to internal RNAses. As such, dsRNA stability may be further increased in killed host cells.

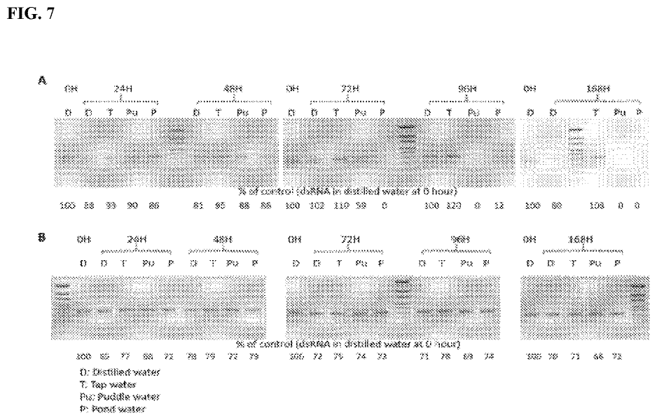

TABLE 3
Environmental stability of naked dsRNA vs dsRNA in bacterial host.
Data summarized from FIG. 7.

| | Percentage of Control (dsRNA at 0 Hr in Distilled Water) | | | | | | | | | |
|---|---|---|---|---|---|---|---|---|---|---|
| Water | 24 Hr | | 48 Hr | | 72 Hr | | 96 Hr | | 168 Hr | |
| Treatment | Naked | C. glutamicum | Naked | C. glutamicum | Naked | C. glutamicum | Naked | C. glutamicum | Naked | C. glutamicum |
| Distilled | 88 | 81.8 | 91 | 77.9 | 102 | 72 | 100 | 71.3 | 80 | 70.3 |
| Tap | 93 | 76.8 | 95 | 79.5 | 110 | 74.6 | 120 | 77.6 | 108 | 71.3 |
| Puddle | 93 | 68 | 38 | 72.2 | 59 | 73.6 | 0 | 69.3 | | 66 |
| Pond | 86 | 72 | 96 | 79.5 | 0 | 72.6 | 12 | 73.5 | | 71.3 |

### Example 15

### Environmental Stability of dsRNA in Corynebacterium glutamicum

The environmental stability of dsRNA in Corynebacterium glutamicum is assessed on a leaf surface. Briefly, naked dsRNA targeting an insect gene and C.g. host cells containing the same dsRNA are placed on a plant leaf for 1, 3 and 6 days at room temperature. At each time point, the amount of dsRNA in each case is assessed by washing off the host cells, extracting dsRNA and quantifying the amount of dsRNA targeting an insect gene by gel, with the amount of dsRNA at 0 hours serving as a control.

What is claimed is:

1. A microorganism genetically modified to control an insect pest, the microorganism comprising:
(i) a promoter operably linked to a DNA sequence comprising a coat protein gene encoding a capsid protein or N-terminus of the capsid protein coding sequence (CDS); and
(ii) a promoter operably linked to a DNA sequence encoding a dsRNA molecule that specifically inhibits expression of a target gene in the insect pest, wherein the dsRNA is at least 60% undegraded dsRNA after exposure of least 7 days to an aqueous environment relative to degradation of the dsRNA when isolated.

2. The microorganism according to claim 1, wherein the DNA sequence encoding a dsRNA molecule encodes a self-complementary stretch of sequence separated by non-complementary sequence such that upon hybridization of the complementary sequences a stem-loop structure is formed, wherein the stem portion of the molecule functions as an RNAi precursor when introduced into the target insect pest.

3. The microorganism according to claim 1, wherein the capsid protein is encoded by a leviviridae coat protein gene.

4. The microorganism according to claim 1, wherein the capsid protein is encoded by the coat protein gene of bacteriophage MS2 or bacteriophage Qβ.

5. The microorganism according to claim 1, wherein the DNA sequence encoding the dsRNA and the coat protein gene encoding the capsid protein are present on one plasmid or extrachromosomal element within the microorganism.

6. The microorganism according to claim 5, wherein the plasmid or extrachromosomal element is pAPSE10180 encoded by SEQ ID NO: 1, pAPSE10269 encoded by SEQ ID NO: 8, pAPSE10216 encoded by SEQ ID NO: 10, pAPSE10219 encoded by SEQ ID NO: 12, pAPSE10279 encoded by SEQ ID NO: 14, pAPSE10270 encoded by SEQ ID NO: 16, pAPSE10271 encoded by SEQ ID NO: 17, pAPSE10272 encoded by SEQ ID NO: 18, pAPSE10292 encoded by SEQ ID NO: 19, pAPSE10291 encoded by SEQ ID NO: 20, pAPSE10359 encoded by SEQ ID NO: 25, pAPSE10372 encoded by SEQ ID NO: 27, pAPSE10429 encoded by SEQ ID NO: 29, pAPSE10430 encoded by SEQ ID NO: 31, pAPSE10431 encoded by SEQ ID NO: 32, pAPSE10432 encoded by SEQ ID NO: 33, pAPSE10434 encoded by SEQ ID NO: 35, pAPSE10436 encoded by SEQ ID NO: 37, pAPSE10440 encoded by SEQ ID NO: 40, pAPSE10442 encoded by SEQ ID NO: 42, pAPSE10444 encoded by SEQ ID NO: 44, or pAPSE10445 encoded by SEQ ID NO: 45.

7. The microorganism according to claim 1, wherein the microorganism is an Escherichia coli strain.

8. The microorganism according to claim 1, wherein the microorganism is a Corynebacterium glutamicum strain.

9. The microorganism according to claim 1, wherein the target gene is beta-actin or the genes coding for inhibitors of apoptosis.

10. The microorganism of claim 1, wherein the body of water is selected from, puddle water, and pond water.

11. The microorganism of claim 1, wherein the target gene is selected from extracellular signal-regulated kinase 1 (erkA), tubulin, vATPase, acetyl choline esterase, chitin synthase gene A, beta-actin, and genes coding for inhibitors of apoptosis.

12. A system for delivering dsRNA to an insect pest, comprising:
(a) water; and
(b) in the water, a genetically modified microorganism comprising:
(i) a promoter operably linked to a DNA sequence comprising a coat protein gene encoding a capsid protein or N-terminus of the capsid protein coding sequence (CDS); and
(ii) a promoter operably linked to a DNA sequence encoding a dsRNA molecule that specifically inhibits expression of a target gene in an insect pest, wherein the dsRNA is at least 60% undegraded after at least 7 days in the water relative to the dsRNA when isolated.

13. The system according to claim 12, wherein the DNA sequence encoding a dsRNA molecule encodes a self-complementary stretch of sequence separated by non-complementary sequence such that upon hybridization of the complementary sequences a stem-loop structure is formed, wherein the stem portion of the molecule functions as an RNAi precursor when introduced into the target insect pest.

14. The system according to claim 12, wherein the capsid protein is encoded by a leviviridae coat protein gene.

15. The system according to claim 12, wherein the capsid protein is encoded by the coat protein gene of bacteriophage MS2 or bacteriophage Qβ.

16. The system according to claim 12, wherein the DNA sequence encoding the dsRNA and the coat protein gene encoding the capsid protein are present on one plasmid or extrachromosomal element within the microorganism.

17. The system according to claim 16, wherein the plasmid or extrachromosomal element is pAPSE10180 encoded by SEQ ID NO: 1, pAPSE10269 encoded by SEQ ID NO: 8, pAPSE10216 encoded by SEQ ID NO: 10, pAPSE10219 encoded by SEQ ID NO: 12, pAPSE10279 encoded by SEQ ID NO: 14, pAPSE10270 encoded by SEQ ID NO: 16, pAPSE10271 encoded by SEQ ID NO: 17, pAPSE10272 encoded by SEQ ID NO: 18, pAPSE10292 encoded by SEQ ID NO: 19, pAPSE10291 encoded by SEQ ID NO: 20, pAPSE10359 encoded by SEQ ID NO: 25, pAPSE10372 encoded by SEQ ID NO: 27, pAPSE10429 encoded by SEQ ID NO: 29, pAPSE10430 encoded by SEQ ID NO: 31, pAPSE10431 encoded by SEQ ID NO: 32, pAPSE10432 encoded by SEQ ID NO: 33, pAPSE10434 encoded by SEQ ID NO: 35, pAPSE10436 encoded by SEQ ID NO: 37, pAPSE10440 encoded by SEQ ID NO: 40, pAPSE10442 encoded by SEQ ID NO: 42, pAPSE10444 encoded by SEQ ID NO: 44, or pAPSE10445 encoded by SEQ ID NO: 45.

18. The system according to claim 12, wherein the microorganism is an Escherichia coli strain or a Corynebacterium glutamicum strain.

19. The system of claim 12, wherein the body of water is selected from, puddle water, and pond water.

20. The system of claim 12, wherein the target gene is selected from extracellular signal-regulated kinase 1 (erkA), tubulin, vATPase, acetyl choline esterase, chitin synthase gene A, beta-actin, and genes coding for inhibitors of apoptosis.
